(19) United States
(12) Patent Application Publication
(10) Pub. No.: US 2025/0295661 A1
(43) Pub. Date: Sep. 25, 2025

(54) MENIN-MLL INHIBITORS FOR THE TREATMENT OF CANCER

(71) Applicant: Syndax Pharmaceuticals, Inc., Waltham, MA (US)

(72) Inventors: Briggs W. MORRISON, Newton, PA (US); Gerard M. MCGEEHAN, Newtown Square, PA (US)

(21) Appl. No.: 18/863,966

(22) PCT Filed: May 9, 2023
(86) PCT No.: PCT/US2023/021604

§ 371 Date: Nov. 7, 2024

Related U.S. Application Data
(60) Provisional application No. 63/339,635, filed on May 9, 2022.

(51) Int. Cl. A61K 31/506; A61K 31/5377; A61K 45/06

(52) U.S. Cl. CPC A61K 31/506; A61K 31/5377; A61K 45/06

(57) ABSTRACT

The present disclosure is directed to methods of treating colorectal cancer in a subject in need thereof with a menin-MLL inhibitor, including Compound A:

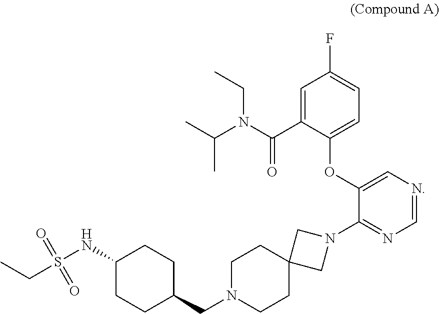 [SMILES: CCN(C(=O)c1cc(F)ccc1Oc1cncnc1N1CC2(CCN(C[C@H]3CC[C@H](NS(=O)(=O)CC)CC3)CC2)C1)C(C)C]

Methods for dosing Compound A for various indications are also provided by the present disclosure.

### RELATED APPLICATION

This application claims priority to, and the benefit of, U.S. Provisional Application No. 63/339,635, filed on May 9, 2022, the content of which is incorporated by reference herein in its entirety for all purposes.

### BACKGROUND

Colorectal cancer generally is a cancer from uncontrolled cell growth in the colon or rectum (parts of the large intestine) or in the appendix. Genetic analyses shows that essentially colon and rectal tumors are genetically the same cancer and symptoms of colorectal cancer typically include rectal bleeding and anemia which are sometimes associated with weight loss and changes in bowel habits. Colorectal cancer (CRC) is a major global health issue, being the third most commonly diagnosed malignancy with an estimated global incidence of over 1.8 million in 2018. CRC is the second commonest cause of global cancer mortality with 0.5 million deaths in 2018. Twenty percent of patients have metastatic colorectal cancer (mCRC) at presentation, whilst up to 50% of the patients who present with early-stage disease relapse later, despite curative-intent surgery, (neo) adjuvant chemotherapy and/or radiotherapy. At least 50% of the Western population will develop a colorectal tumor by age 70 years. In 10% of these individuals, the tumor progresses to malignancy. In adults, colorectal cancer is the second leading cancer that causes death worldwide. Accordingly, clinically efficacious treatments are severely lacking, and the present disclosure aims to satisfy this presently unmet clinical need.

### SUMMARY

In some aspects, the present disclosure is directed to a method of treating, preventing, or reducing the severity of a colorectal cancer in a subject in need thereof, comprising administering to the subject an effective amount of a menin-MLL inhibitor or a pharmaceutical composition comprising a menin-MLL inhibitor.

In some aspects, the present disclosure is directed to a method of treating, preventing, or reducing the severity of a colorectal cancer in a subject in need thereof, comprising administering to the subject a pharmaceutical composition consisting essentially of a menin-MLL inhibitor.

In some aspects, the present disclosure is directed to a method of treating, preventing, or reducing the severity of a colorectal cancer in a subject in need thereof consisting essentially of administering to the subject a pharmaceutical composition comprising a menin-MLL inhibitor.

In some aspects, the present disclosure is directed to a method of treating, preventing, or reducing the severity of colorectal cancer in a subject in need thereof, comprising administering to the subject an effective amount of a single anti-cancer agent, wherein the single anti-cancer agent is a menin-MLL inhibitor.

In some aspects, the present disclosure is directed to a method of treating, preventing, or reducing the severity of a colorectal cancer in a subject in need thereof comprising administering:
a pharmaceutical composition comprising Compound A at least once daily, and
a pharmaceutical composition comprising cobicistat at least once daily.

In some aspects, the present disclosure is directed to a method of treating, preventing, or reducing the severity of a colorectal cancer in a subject in need thereof comprising administering:
a pharmaceutical composition comprising Compound A at least once daily,
a pharmaceutical composition comprising cobicistat at least once daily, and
wherein the method does not involve administering an EGFR inhibitor.

In some aspects, the present disclosure is directed to a method of treating, preventing, or reducing the severity of a colorectal cancer in a subject in need thereof comprising administering:
a pharmaceutical composition comprising Compound A at least once daily,
a pharmaceutical composition comprising cobicistat at least once daily, and
wherein the subject has not received an EGFR inhibitor.

In some aspects, the present disclosure is directed to a method of treating, preventing, or reducing the severity of a colorectal cancer in a subject in need thereof comprising administering:
a pharmaceutical composition comprising Compound A at least once daily,
a pharmaceutical composition comprising cobicistat at least once daily, and
wherein the subject is not also receiving an EGFR inhibitor.

In some aspects, the present disclosure is directed to a method of treating, preventing, or reducing the severity of a colorectal cancer in a subject in need thereof comprising administering:
a pharmaceutical composition comprising Compound A at least once daily,
a pharmaceutical composition comprising cobicistat at least once daily, and
wherein the method further comprises administering Compound A in the absence of an EGFR inhibitor.

In some aspects, the present disclosure is directed to a method of treating, preventing, or reducing the severity of a colorectal cancer in a subject in need thereof comprising administering Compound A at a dose between about 25 mg and about 300 mg, wherein the subject does not receive a CYP3A inhibitor.

In some aspects, the present disclosure is directed to a method of treating, preventing, or reducing the severity of a colorectal cancer in a subject in need thereof comprising administering Compound A at a dose between about 25 mg and about 300 mg, wherein the subject is not also receiving a CYP3A inhibitor.

In some aspects, the present disclosure is directed to a method of treating, preventing, or reducing the severity of a colorectal cancer in a subject in need thereof comprising administering Compound A at a dose between about 25 mg and about 300 mg, wherein the method does not involve administering a CYP3A inhibitor.

In some aspects, the present disclosure is directed to a method of treating, preventing, or reducing the severity of a colorectal cancer in a subject in need thereof comprising administering Compound A at a dose between about 25 mg and about 300 mg, wherein administering Compound A occurs in the absence of a CYP3A inhibitor.

In some aspects, the present disclosure is directed to a method of treating, preventing, or reducing the severity of a colorectal cancer in a subject in need thereof comprising administering Compound A at a dose between about 25 mg and about 300 mg,
wherein administering Compound A occurs in the absence of both a CYP3A inhibitor and an EGFR inhibitor.

In some aspects, the present disclosure is directed to a method of treating, preventing, or reducing the severity of a colorectal cancer in a subject in need thereof comprising administering Compound A at a dose between about 25 mg and about 300 mg at least once daily, wherein the subject does not receive a CYP3A inhibitor.

In some aspects, the present disclosure is directed to a method of treating, preventing, or reducing the severity of a colorectal cancer in a subject in need thereof comprising administering Compound A at a dose between about 25 mg and about 300 mg at least once daily, wherein the subject is not also receiving a CYP3A inhibitor.

In some aspects, the present disclosure is directed to a method of treating, preventing, or reducing the severity of a colorectal cancer in a subject in need thereof comprising administering Compound A at a dose between about 25 mg and about 300 mg at least once daily, wherein the method does not involve administering a CYP3A inhibitor.

In some aspects, the present disclosure is directed to a method of treating, preventing, or reducing the severity of a colorectal cancer in a subject in need thereof comprising administering Compound A at a dose between about 25 mg and about 300 mg at least once daily, wherein administering Compound A occurs in the absence of a CYP3A inhibitor.

In some aspects, the present disclosure is directed to a method of treating, preventing, or reducing the severity of a colorectal cancer in a subject in need thereof comprising administering Compound A at a dose between about 25 mg and about 300 mg at least once daily,
wherein administering Compound A occurs in the absence of both a CYP3A inhibitor and an EGFR inhibitor.

In some aspects, the present disclosure is directed to a method of treating, preventing, or reducing the severity of a colorectal cancer in a subject in need thereof comprising administering Compound A at a dose between about 150 mg and about 200 mg, wherein the subject does not receive a CYP3A inhibitor.

In some aspects, the present disclosure is directed to a method of treating, preventing, or reducing the severity of a colorectal cancer in a subject in need thereof comprising administering Compound A at a dose between about 150 mg and about 200 mg, wherein the subject is not also receiving a CYP3A inhibitor.

In some aspects, the present disclosure is directed to a method of treating, preventing, or reducing the severity of a colorectal cancer in a subject in need thereof comprising administering Compound A at a dose between about 150 mg and about 200 mg, wherein the method does not involve administering a CYP3A inhibitor.

In some aspects, the present disclosure is directed to a method of treating, preventing, or reducing the severity of a colorectal cancer in a subject in need thereof comprising administering Compound A at a dose between about 150 mg and about 200 mg, wherein administering Compound A occurs in the absence of a CYP3A inhibitor.

In some aspects, the present disclosure is directed to a method of treating, preventing, or reducing the severity of a colorectal cancer in a subject in need thereof comprising administering Compound A at a dose between about 150 mg and about 200 mg,
wherein administering Compound A occurs in the absence of both a CYP3A inhibitor and an EGFR inhibitor.

In some aspects, the present disclosure is directed to a method of treating, preventing, or reducing the severity of a cancer comprising administering a pharmaceutical composition comprising Compound A, wherein the method comprises administering Compound A in the absence of a CYP3A inhibitor, wherein the dose of Compound A is between about 220 and about 280 mg.

In some aspects, the present disclosure is directed to a method of treating, preventing, or reducing the severity of a colorectal cancer in a subject in need thereof comprising administering Compound A at a dose between about 150 mg and about 200 mg at least once daily, wherein the subject does not receive a CYP3A inhibitor.

In some aspects, the present disclosure is directed to a method of treating, preventing, or reducing the severity of a colorectal cancer in a subject in need thereof comprising administering Compound A at a dose between about 150 mg and about 200 mg at least once daily, wherein the subject is not also receiving a CYP3A inhibitor.

In some aspects, the present disclosure is directed to a method of treating, preventing, or reducing the severity of a colorectal cancer in a subject in need thereof comprising administering Compound A at a dose between about 150 mg and about 200 mg at least once daily, wherein the method does not involve administering a CYP3A inhibitor.

In some aspects, the present disclosure is directed to a method of treating, preventing, or reducing the severity of a colorectal cancer in a subject in need thereof comprising administering Compound A at a dose between about 150 mg and about 200 mg at least once daily, wherein administering Compound A occurs in the absence of a CYP3A inhibitor.

In some aspects, the present disclosure is directed to a method of treating, preventing, or reducing the severity of a colorectal cancer in a subject in need thereof comprising administering Compound A at a dose between about 150 mg and about 200 mg at least once daily,
wherein administering Compound A occurs in the absence of both a CYP3A inhibitor and an EGFR inhibitor.

In some aspects, the present disclosure is directed to a method of treating, preventing, or reducing the severity of a cancer comprising administering a pharmaceutical composition comprising Compound A, wherein the method comprises administering Compound A in the absence of a CYP3A inhibitor, wherein the dose of Compound A is between about 220 and about 280 mg, and wherein Compound A is administered at least once per day.

In some aspects, the present disclosure is directed to a method of treating, preventing, or reducing the severity of a colorectal cancer in a subject in need thereof comprising administering Compound A at a dose between about 150 mg and about 200 mg three times daily, wherein the subject does not receive a CYP3A inhibitor.

In some aspects, the present disclosure is directed to a method of treating, preventing, or reducing the severity of a colorectal cancer in a subject in need thereof comprising administering Compound A at a dose between about 150 mg and about 200 mg three times daily, wherein the subject is not also receiving a CYP3A inhibitor.

In some aspects, the present disclosure is directed to a method of treating, preventing, or reducing the severity of a colorectal cancer in a subject in need thereof comprising administering Compound A at a dose between about 150 mg and about 200 mg three times daily, wherein the method does not involve administering a CYP3A inhibitor.

In some aspects, the present disclosure is directed to a method of treating, preventing, or reducing the severity of a colorectal cancer in a subject in need thereof comprising administering Compound A at a dose between about 150 mg and about 200 mg three times daily, wherein administering Compound A occurs in the absence of a CYP3A inhibitor.

In some aspects, the present disclosure is directed to a method of treating, preventing, or reducing the severity of a colorectal cancer in a subject in need thereof comprising administering Compound A at a dose between about 150 mg and about 200 mg three times daily,
wherein administering Compound A occurs in the absence of both a CYP3A inhibitor and an EGFR inhibitor.

In some aspects, the present disclosure is directed to a method of treating, preventing, or reducing the severity of a colorectal cancer in a subject in need thereof comprising administering Compound A, wherein the subject has progressed on a prior colorectal cancer therapy.

In some aspects, the present disclosure is directed to a method of treating, preventing, or reducing the severity of a colorectal cancer in a subject in need thereof comprising administering Compound A, wherein the subject has relapsed from at least one prior line of therapy for colorectal cancer.

In some aspects, the present disclosure is directed to a method of treating, preventing, or reducing the severity of a colorectal cancer in a subject in need thereof comprising administering Compound A, wherein the subject has relapsed from at least two prior lines of therapy for colorectal cancer.

In some aspects, the present disclosure is directed to a method of treating, preventing, or reducing the severity of a colorectal cancer in a subject in need thereof comprising administering Compound A, wherein the subject has refractory colorectal cancer.

In some aspects, the present disclosure is directed to a method of treating, preventing, or reducing the severity of a cancer comprising administering a pharmaceutical composition comprising Compound A, wherein the method comprises administering Compound A in the absence of a CYP3A inhibitor, wherein the dose of Compound A is between about 220 and about 280 mg, and wherein Compound A is administered three times per day.

In some aspects, the present disclosure is directed to a method of treating, preventing, or reducing the severity of a colorectal cancer in a subject in need thereof comprising administering Compound A at a daily dose of between about 25 mg and about 900 mg, wherein the subject does not receive a CYP3A inhibitor.

In some aspects, the present disclosure is directed to a method of treating, preventing, or reducing the severity of a colorectal cancer in a subject in need thereof comprising administering Compound A at a daily dose of between about 25 mg and about 900 mg, wherein the subject is not also receiving a CYP3A inhibitor.

In some aspects, the present disclosure is directed to a method of treating, preventing, or reducing the severity of a colorectal cancer in a subject in need thereof comprising administering Compound A at a daily dose of between about 25 mg and about 900 mg, wherein the method does not involve administering a CYP3A inhibitor.

In some aspects, the present disclosure is directed to a method of treating, preventing, or reducing the severity of a colorectal cancer in a subject in need thereof comprising administering Compound A at a daily dose of between about 25 mg and about 900 mg, wherein administering Compound A occurs in the absence of a CYP3A inhibitor.

In some aspects, the present disclosure is directed to a method of treating, preventing, or reducing the severity of a colorectal cancer in a subject in need thereof comprising administering Compound A at a daily dose of between about 25 mg and about 900 mg,
wherein administering Compound A occurs in the absence of both a CYP3A inhibitor and an EGFR inhibitor.

In some aspects, the present disclosure is directed to a menin-MLL inhibitor for use in the preparation of a medicament for the treatment of colorectal cancer according to the method of any one of the previous embodiments, wherein the medicament and/or treatment does not involve the use of an EGFR inhibitor.

In some aspects, the present disclosure is directed to a menin-MLL inhibitor for use in the preparation of a medicament for the treatment of colorectal cancer according to the method of any one of the previous embodiments, wherein the medicament does not comprise an EGFR inhibitor.

In some aspects, the present disclosure is directed to a kit comprising a menin-MLL inhibitor, and printed instructions for using the menin-MLL inhibitor in a subject for the treatment for colorectal cancer.

### BRIEF DESCRIPTION OF THE FIGURES

The accompanying drawings, which are incorporated into and form a part of the specification, illustrate several embodiments of the present disclosure and, together with the description, serve to explain the principles of the present disclosure. The drawings are only for the purpose of illustrating an embodiment of the disclosure and are not to be construed as limiting the invention. Further objects, features and advantages of the invention will become apparent from the following detailed description taken in conjunction with the accompanying figures showing illustrative embodiments of the disclosure.

FIG. 1 depicts an overview of the clinical trial protocol of the use of a menin-MLL inhibitor, Compound A, for the treatment of colorectal cancer.

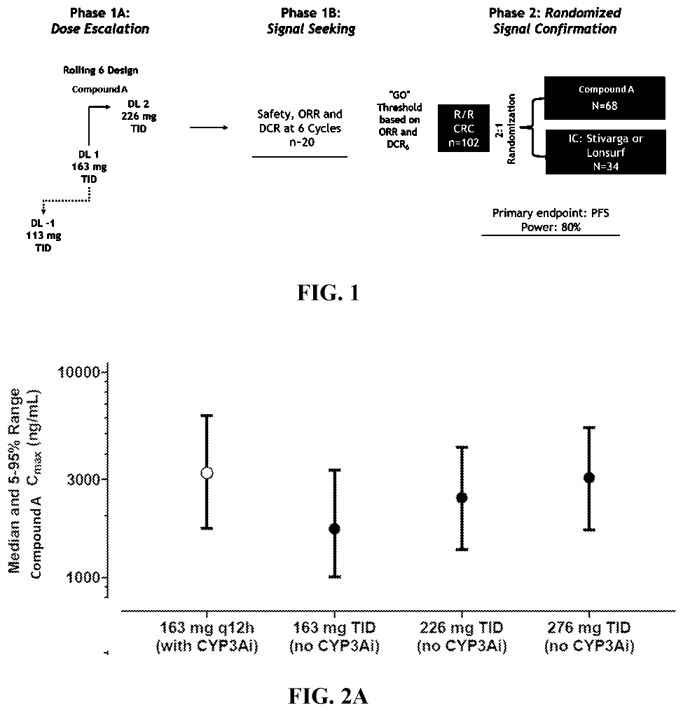

FIGS. 2A-2C depict the simulated median and 5-95% pharmacokinetic parameters for 163 mg of Compound A given every 12 hours (q12 h) with a strong CYP3A inhibitor, compared to 163 mg, 226 mg, or 276 mg of Compound A given three times per day (TID) without a CYP3A inhibitor. FIG. 2A depicts Compound A Cₘₐₓ. FIG. 2B depicts the Compound A Cₘᵢₙ. FIG. 2C depicts the Compound A AUC₀₋₂₄.

### DETAILED DESCRIPTION

The following is a detailed description provided to aid those skilled in the art in practicing the present invention. Those of ordinary skill in the art may make modifications and variations in the embodiments described herein without departing from the spirit or scope of the present disclosure. All publications, patent applications, patents, figures and other references mentioned herein are expressly incorporated by reference in their entirety.

In some aspects, the present disclosure is directed to a method of treating, preventing, or reducing the severity of a colorectal cancer in a subject in need thereof, comprising administering to the subject a menin-MLL inhibitor, or pharmaceutical composition thereof.

In some aspects, the present disclosure is directed to a method of treating, preventing, or reducing the severity of a colorectal cancer in a subject in need thereof, comprising administering to the subject a pharmaceutical composition consisting essentially of a menin-MLL inhibitor.

In some aspects, the present disclosure is directed to a method of treating, preventing, or reducing the severity of a colorectal cancer in a subject in need thereof consisting essentially of administering to the subject a pharmaceutical composition comprising a menin-MLL inhibitor.

In some aspects, the present disclosure is directed to a method of treating, preventing, or reducing the severity of colorectal cancer in a subject in need thereof, comprising administering to the subject a single anti-cancer agent, wherein the single anti-cancer agent is a menin-MLL inhibitor.

### Menin-MLL Inhibitors

In some embodiments, the menin-MLL inhibitor is Compound B, KO-539, JNJ-75276617, Compound A, DS-1594, BMF-219, DSP-5336, the antibody A300-105A (commercially available from Bethyl Laboratories), MI-0202, MI-503, MI-463, MI-136, MI-3454, M-808 and ML-227, or a pharmaceutically acceptable salt thereof.

In some embodiments, the menin-MLL inhibitor is a compound of Table 1

TABLE 1
Menin-MLL inhibitors

| | | | |
|---|---|---|---|
| Compound A | 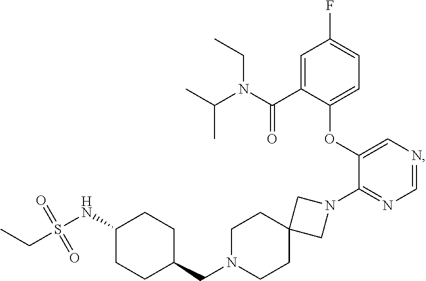 [SMILES: CCN(C(=O)c1cc(F)ccc1Oc1cncnc1N1CC2(CCN(C[C@H]3CC[C@H](NS(=O)(=O)CC)CC3)CC2)C1)C(C)C] | DSP- 5336 | 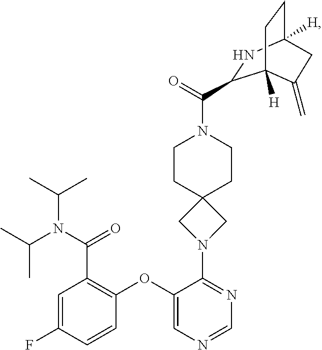 |
| Compound B | 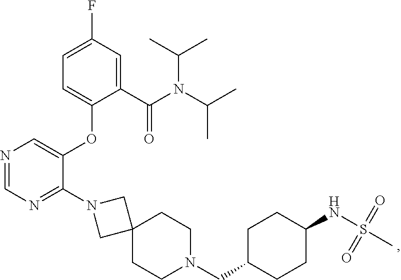 [SMILES: CC(C)N(C(=O)c1cc(F)ccc1Oc1cncnc1N1CC2(CCN(C[C@H]3CC[C@H](NS(C)(=O)=O)CC3)CC2)C1)C(C)C] | DS- 1594 | 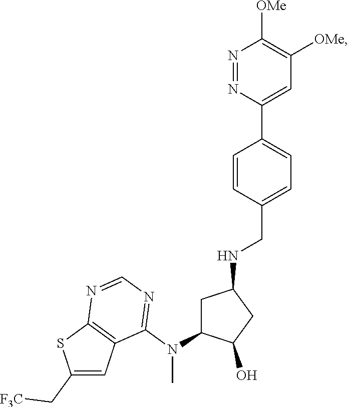 [SMILES: COc1cc(-c2ccc(CN[C@H]3C[C@@H](O)[C@@H](N(C)c4ncnc5sc(CC(F)(F)F)cc45)C3)cc2)nnc1OC] |
| MI-463 | 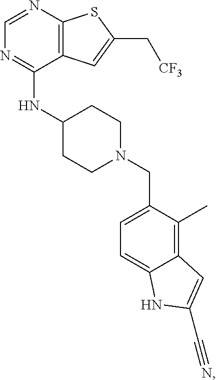 [SMILES: Cc1c(CN2CCC(Nc3ncnc4sc(CC(F)(F)F)cc34)CC2)ccc2[nH]c(C#N)cc12] | MI-503 | 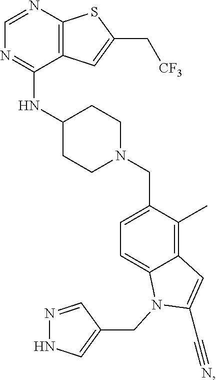 [SMILES: Cc1c(CN2CCC(Nc3ncnc4sc(CC(F)(F)F)cc34)CC2)ccc2c1cc(C#N)n2Cc1cn[nH]c1] |
| JNJ- 75276617 | 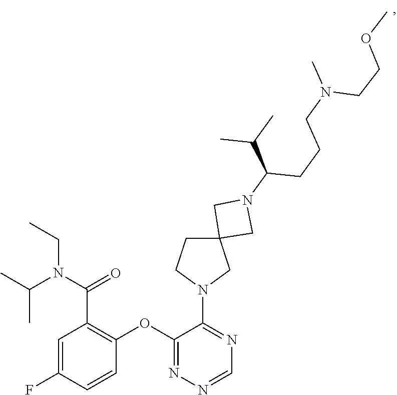 [SMILES: CCN(C(=O)c1cc(F)ccc1Oc1nncnc1N1CCC2(C1)CN([C@H](CCCN(C)CCOC)C(C)C)C2)C(C)C] | MI- 136 | 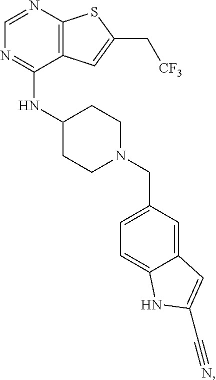 [SMILES: N#Cc1cc2cc(CN3CCC(Nc4ncnc5sc(CC(F)(F)F)cc45)CC3)ccc2[nH]1] |

| | |
|---|---|
| M-808 | 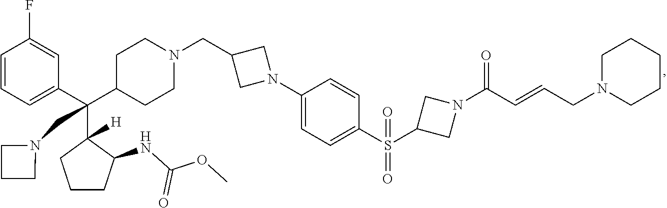 |
| MI-227 | 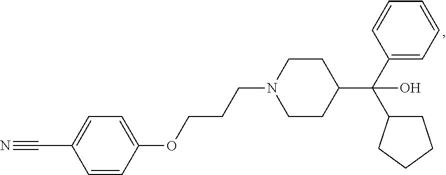 [SMILES: N#Cc1ccc(OCCCN2CCC(C(O)(c3ccccc3)C3CCCC3)CC2)cc1] |
| KO-539 | 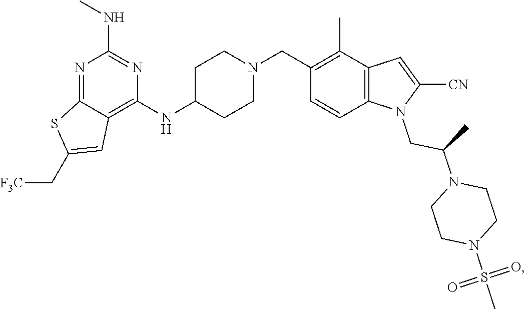 |
| MI-3454 | 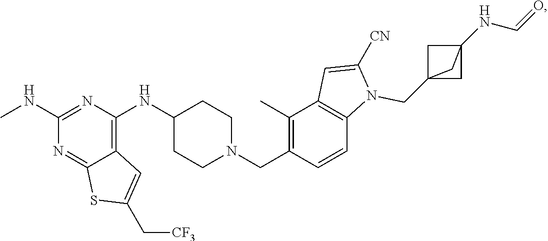 [SMILES: CNc1nc(NC2CCN(Cc3ccc4c(cc(C#N)n4CC45CC(NC=O)(C4)C5)c3C)CC2)c2cc(CC(F)(F)F)sc2n1] |
| BMF-219 | 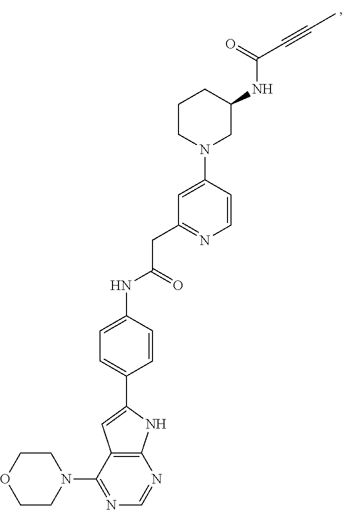 [SMILES: CC#CC(=O)N[C@@H]1CCCN(c2ccnc(CC(=O)Nc3ccc(-c4cc5c(N6CCOCC6)ncnc5[nH]4)cc3)c2)C1] |

or a pharmaceutically acceptable salt thereof.

Another menin-MLL inhibitor is MI-2-2, which inhibits the interaction between menin and an MLL. See, Grembecka et al., 2012, Nat Chem Biol, 8:277-284; Shi et al., 2012, Blood, 120:4461-4469. Yet another menin-MLL inhibitor, which inhibits the interaction of an MLL enzyme and menin, is N,N′-bis(4-aminophenyl)-N,N′-dimethylethylenediamine (also known as ISC-30). In some embodiments, menin-MLL inhibitors that contemplated for use in the methods of the present disclosure are known in the art at least e.g., WO 2017/112768, WO 2017/214367, WO 2018/053267, WO 2020/069027, WO 2021/207335, US 2021/0115018, US 2019/0307750, US 2016/0339035, and Borkin et al. Cancer cell vol. 27, 4 (2015): 589-602, the contents of each of which are incorporated by reference.

In some embodiments, the menin-MLL inhibitor is Compound A, Compound B, DSP-5336, DS-1594, KO-539, MI-503, MI-3454, MI-463, M-808, MI-136, JNJ-75276617, MI-227, BMF-219, the antibody A300-105A, MI-0202, MI-503, MI-463, MI-136, ML-227, MI-2-2, or ISC-30. In some embodiments, the menin-MLL inhibitor is Compound A or Compound B. In some embodiments, the menin-MLL inhibitor is Compound A. In some embodiments, the menin-MLL inhibitor is DSP-5336. In some embodiments, the menin-MLL inhibitor is Compound B. In some embodiments, the menin-MLL inhibitor is DS-1594. In some embodiments, the menin-MLL inhibitor is KO-539. In some embodiments, the menin-MLL inhibitor is MI-503. In some embodiments, the menin-MLL inhibitor MI-3454. In some embodiments, the menin-MLL inhibitor is MI-463. In some embodiments, the menin-MLL inhibitor is M-808. In some embodiments, the menin-MLL inhibitor is MI-136. In some embodiments, the menin-MLL inhibitor is JNJ-75276617. In some embodiments, the menin-MLL inhibitor is MI-227. In some embodiments, the menin-MLL inhibitor is BMF-219. In some embodiments, the menin-MLL inhibitor is the antibody A300-105A. In some embodiments, the menin-MLL inhibitor is MI-0202. In some embodiments, the menin-MLL inhibitor is MI-503. In some embodiments, the menin-MLL inhibitor is MI-463. In some embodiments, the menin-MLL inhibitor is MI-136. In some embodiments, the menin-MLL inhibitor is ML-227. In some embodiments, the menin-MLL inhibitor is MI-2-2. In some embodiments, the menin-MLL inhibitor is ISC-30.

### Methods of Treatment

It is to be understood that, unless otherwise stated, any description of a method of treatment includes use of the compounds to provide such treatment or prophylaxis as is described herein, as well as use of the compounds to prepare a medicament to treat or prevent such condition. The treatment includes treatment of human or non-human animals including rodents and other disease models.

In some embodiments, the menin-MLL inhibitor is administered at least once daily. In some embodiments, the menin-MLL inhibitor is Compound A and is administered at least once daily.

In some embodiments, the menin-MLL inhibitor is administered twice daily. In some embodiments, the menin-MLL inhibitor is Compound A and is administered at least twice daily. In some embodiments, the menin-MLL inhibitor is Compound A and is administered twice daily.

In some embodiments, the menin-MLL inhibitor is administered three times daily. In some embodiments, the menin-MLL inhibitor is Compound A and is administered three times daily.

In some embodiments, the menin-MLL inhibitor is administered in combination with a CYP3A inhibitor.

In some embodiments, a pharmaceutical composition comprising a menin-MLL inhibitor further comprises a CYP3A inhibitor.

In some embodiments, the menin-MLL inhibitor is administered in combination with a CYP3A4 inhibitor.

In some embodiments, a pharmaceutical composition comprising a menin-MLL inhibitor further comprises a CYP3A4 inhibitor.

In some embodiments, the CYP3A4 inhibitor is a strong CYP3A4 inhibitor.

In some embodiments, the strong CYP3A4 inhibitor is boceprevir, cobicistat, clarithromycin, conivaptan, grapefruit juice, indinavir, itraconazole, ketoconazole, lopinavir, mibefradil, nefazodone, nelfinavir, posaconazole, ritonavir, saquinavir, telaprevir, telithromycin, or voriconazole.

In some embodiments, the method further comprises administering cobicistat.

In some embodiments, the method further comprises administering cobicistat at least once daily.

In some embodiments, the method further comprises administering cobicistat once daily.

In some embodiments, the CYP3A4 inhibitor is a moderate CYP3A4 inhibitor.

In some embodiments, the moderate CYP3A4 inhibitor is amprenavir, aprepitant, atazanavir, ciprofloxacin, darunavir, diltiazem, erythromycin, fluconazole, fosamprenavir, imatinib, or verapamil.

In some embodiments, the menin-MLL inhibitor is administered at a dosage of between about 25 mg and about 276 mg every 12 hours.

In some embodiments, the menin-MLL inhibitor is administered at a dosage of about 163 mg every 12 hours.

In some embodiments, the menin-MLL inhibitor is administered to a subject in need thereof in the absence of a CYP3A inhibitor.

In some embodiments, the menin-MLL inhibitor is administered to a subject in need thereof wherein the subject does not receive a CYP3A inhibitor.

In some embodiments, the menin-MLL inhibitor is administered to a subject in need thereof in the absence of a CYP3A4 inhibitor.

In some embodiments, the menin-MLL inhibitor is administered to a subject in need thereof wherein the subject does not receive a CYP3A4 inhibitor.

In some embodiments, the pharmaceutical composition described herein comprises a menin inhibitor (e.g. Compound A) and does not comprise a CYP3A4 inhibitor.

In some embodiments, the menin-MLL inhibitor is administered to a subject in need thereof in an amount between about 25 mg and about 900 mg.

In some embodiments, the menin-MLL inhibitor is administered to a subject in need thereof at a dosage of between about 25 mg and about 276 mg.

In some embodiments, the menin-MLL inhibitor is administered to a subject in need thereof at least one time per day and wherein the total amount of daily administered menin-MLL inhibitor is between about 25 mg and about 900 mg.

In some embodiments, the menin-MLL inhibitor is administered to a subject in need thereof once per day and wherein the total amount of daily administered menin-MLL inhibitor is between about 25 mg and about 900 mg.

In some embodiments, the menin-MLL inhibitor is administered to a subject in need thereof twice per day and wherein the total amount of daily administered menin-MLL inhibitor is between about 25 mg and about 900 mg.

In some embodiments, the menin-MLL inhibitor is administered to a subject in need thereof three times per day and wherein the total amount of daily administered menin-MLL inhibitor is between about 25 mg and about 900 mg.

In some embodiments, the menin-MLL inhibitor is administered to a subject in need thereof four times per day and wherein the total amount of daily administered menin-MLL inhibitor is between about 25 mg and about 900 mg.

In some embodiments, the menin-MLL inhibitor is administered to a subject in need thereof at a dosage of between about 25 mg and about 276 mg, wherein the menin-MLL inhibitor is administered at least one per day.

In some embodiments, the menin-MLL inhibitor is administered to a subject in need thereof at a dosage of between about 25 mg and about 276 mg, wherein the menin-MLL inhibitor is administered one per day.

In some embodiments, the menin-MLL inhibitor is administered to a subject in need thereof at a dosage of between about 25 mg and about 276 mg, wherein the menin-MLL inhibitor is administered twice per day.

In some embodiments, the menin-MLL inhibitor is administered to a subject in need thereof at a dosage of between about 25 mg and about 276 mg, wherein the menin-MLL inhibitor is administered three times per day.

In some embodiments, the menin-MLL inhibitor is administered to a subject in need thereof at a dosage of about 163 mg.

In some embodiments, the menin-MLL inhibitor is administered to a subject in need thereof at a dosage of about 163 mg when the menin-MLL inhibitor is administered three times per day.

In some embodiments, the menin-MLL inhibitor is administered to a subject in need thereof at a dosage of between about 25 mg and about 276 mg.

In some embodiments, the menin-MLL inhibitor is administered to a subject in need thereof in the absence of a CYP3A4 inhibitor and the menin-MLL inhibitor is administered at a dosage of between about 25 mg and about 276 mg, wherein the menin-MLL inhibitor is administered three times per day.

In some embodiments, the menin-MLL inhibitor is administered to a subject in need thereof in the absence of a CYP3A4 inhibitor and the menin-MLL inhibitor is administered at a dosage of about 163 mg.

In some embodiments, the menin-MLL inhibitor is administered to a subject in need thereof in the absence of a CYP3A4 inhibitor and the menin-MLL inhibitor is administered at a dosage of about 163 mg, wherein the menin-MLL inhibitor is administered three times per day.

In some embodiments, the menin-MLL inhibitor is administered to a subject in need thereof in the absence of a CYP3A4 inhibitor and the menin-MLL inhibitor is administered at a dosage of about 276 mg.

In some embodiments, the menin-MLL inhibitor is administered to a subject in need thereof in the absence of a CYP3A4 inhibitor and the menin-MLL inhibitor is administered at a dosage of about 276 mg, wherein the menin-MLL inhibitor is administered three times per day.

In some embodiments, the menin-MLL inhibitor is administered to a subject in need thereof three times per day at a dosage of between about 25 mg and about 276 mg, wherein a CYP3A4 inhibitor is not administered to the subject.

In some embodiments, the menin-MLL inhibitor is administered to a subject in need thereof at about a 163 mg dose, wherein a CYP3A4 inhibitor is not administered to the subject.

In some embodiments, the menin-MLL inhibitor is administered to a subject in need thereof three times per day at about a 163 mg dose, wherein a CYP3A4 inhibitor is not administered to the subject.

In some embodiments, the menin-MLL inhibitor is administered to a subject in need thereof at about a 276 mg dose, wherein a CYP3A4 inhibitor is not administered to the subject.

In some embodiments, the menin-MLL inhibitor is administered to a subject in need thereof three times per day at about a 276 mg dose, wherein a CYP3A4 inhibitor is not administered to the subject.

In some embodiments, the menin-MLL inhibitor is administered to a subject in need thereof at a dosage of up to about 828 mg per day.

In some embodiments, the menin-MLL inhibitor is administered to a subject in need thereof at a dosage of up to about 828 mg per day, wherein a CYP3A inhibitor is not administered to the subject.

In some embodiments, the menin-inhibitor is contained within a capsule or oral solution.

In some embodiments, the subject has not received a prior therapy for colorectal cancer.

In some embodiments, the subject previously received at least one prior therapy for colorectal cancer.

In some embodiments, the subject previously received at least one prior therapy for colorectal cancer, wherein the subject did not respond to the at least one prior line of therapy.

In some embodiments, the at least one prior line of therapy was a chemotherapy, radiation therapy, immunotherapy, targeted small molecule therapy or biologic therapy (e.g. monoclonal antibody therapy). In some embodiments, the at least one prior line of therapy was a chemotherapy. In some embodiments, the at least one prior line of therapy was a radiation therapy. In some embodiments, the at least one prior line of therapy is an immunotherapy. In some embodiments, the at least one prior line of therapy is a targeted small molecule therapy. In some embodiments, the at least one prior line of therapy is biologic therapy (e.g. monoclonal antibody therapy).

In some embodiments, the subject has previously received a treatment for colorectal cancer selected from one or more of trifluridine, tipiracil and or regorafenib.

In some embodiments, the subject has previously received trifluridine, tipiracil, or a combination thereof for the treatment of colorectal cancer.

In some embodiments, the subject has previously received regorafenib for the treatment of colorectal cancer.

In some embodiments, the subject previously received trifluridine, tipiracil and/or regorafenib. In some embodiments, the subject previously received trifluridin. In some embodiments, the subject previously received tipiracil. In some embodiments, the subject previously received regorafenib.

In some embodiments, the subject has not received a prior therapy for colorectal cancer.

In some embodiments, the subject progressed from at least one prior line of therapy for colorectal cancer. In some embodiments, the subject relapsed from at least one prior line of therapy for colorectal cancer. In some embodiments, the subject relapsed from at least two prior therapies for colorectal cancer. In some embodiments, the subject has refractory colorectal cancer. In some embodiments, the subject has relapsed or refractory colorectal cancer.

In some embodiments, the colorectal cancer is relapsed/refractory colorectal cancer. In some embodiments, the colorectal cancer is metastatic relapsed/refractory colorectal cancer.

In some embodiments, the subject is being treated for microsatellite stable/proficient mismatch repair (MSS/pMMR) metastatic colorectal cancer (mCRC). In some embodiments, the colorectal cancer is microsatellite stable/proficient mismatch repair (MSS/pMMR) metastatic colorectal cancer.

In some embodiments, the menin-MLL inhibitor is administered in the absence of an EGFR inhibitor.

In some aspects, the present disclosure is directed to a method of treating, preventing, or reducing the severity of a colorectal cancer in a subject in need thereof comprising administering:
a pharmaceutical composition comprising a menin-MLL inhibitor at least once daily, and
a pharmaceutical composition comprising a CYP3A4 inhibitor at least once daily.

In some aspects, the present disclosure is directed to a method of treating, preventing, or reducing the severity of a colorectal cancer in a subject in need thereof comprising administering:
a pharmaceutical composition comprising a menin-MLL inhibitor at least once daily,
a pharmaceutical composition comprising a CYP3A4 inhibitor at least once daily, and
wherein the method does not involve administering an EGFR inhibitor.

In some aspects, the present disclosure is directed to a method of treating, preventing, or reducing the severity of a colorectal cancer in a subject in need thereof comprising administering:
a pharmaceutical composition comprising a menin-MLL inhibitor at least once daily,
a pharmaceutical composition comprising a CYP3A4 inhibitor at least once daily, and
wherein the subject has not received an EGFR inhibitor.

In some aspects, the present disclosure is directed to a method of treating, preventing, or reducing the severity of a colorectal cancer in a subject in need thereof comprising administering:
a pharmaceutical composition comprising a menin-MLL inhibitor at least once daily,
a pharmaceutical composition comprising a CYP3A4 inhibitor at least once daily, and
wherein the subject is not also receiving an EGFR inhibitor.

In some aspects, the present disclosure is directed to a method of treating, preventing, or reducing the severity of a colorectal cancer in a subject in need thereof comprising administering:
a pharmaceutical composition comprising a menin-MLL inhibitor at least once daily,
a pharmaceutical composition comprising a CYP3A4 inhibitor at least once daily, and
wherein the method further comprises administering a menin-MLL inhibitor in the absence of an EGFR inhibitor.

In some aspects, the present disclosure is directed to a method of treating, preventing, or reducing the severity of a colorectal cancer in a subject in need thereof comprising administering:
a pharmaceutical composition comprising a menin-MLL inhibitor at least once daily,
a pharmaceutical composition comprising a CYP3A4 inhibitor at least once daily, and
wherein the method further comprises administering the menin-MLL inhibitor in the absence of an EGFR inhibitor.

In some aspects, the present disclosure is directed to a method of treating, preventing, or reducing the severity of a colorectal cancer in a subject in need thereof comprising administering:
a pharmaceutical composition comprising a menin-MLL inhibitor at least once daily, and
a pharmaceutical composition comprising a CYP3A4 inhibitor at least once daily, and wherein the menin-MLL inhibitor selected from the group consisting of Compound B, KO-539, JNJ-75276617, Compound-A, DS-1594, BMF-219, DSP-5336, the antibody A300-105A (commercially available from Bethyl Laboratories), MI-0202, MI-503, MI-463, MI-136, MI-3454, M-808 and ML-227, or a pharmaceutically acceptable salt thereof.

In some aspects, the present disclosure is directed to a method of treating, preventing, or reducing the severity of a colorectal cancer in a subject in need thereof comprising administering:
a pharmaceutical composition comprising a menin-MLL inhibitor at least once daily,
a pharmaceutical composition comprising a CYP3A4 inhibitor at least once daily, and wherein the method does not involve administering an EGFR inhibitor, and wherein the menin-MLL inhibitor selected from the group consisting of Compound B, KO-539, JNJ-75276617, Compound-A, DS-1594, BMF-219, DSP-5336, the antibody A300-105A (commercially available from Bethyl Laboratories), MI-0202, MI-503, MI-463, MI-136, MI-3454, M-808 and ML-227, or a pharmaceutically acceptable salt thereof.

In some aspects, the present disclosure is directed to a method of treating, preventing, or reducing the severity of a colorectal cancer in a subject in need thereof comprising administering:
a pharmaceutical composition comprising a menin-MLL inhibitor at least once daily,
a pharmaceutical composition comprising a CYP3A4 inhibitor at least once daily, and wherein the subject has not received an EGFR inhibitor, and wherein the menin-MLL inhibitor selected from the group consisting of Compound B, KO-539, JNJ-75276617,
Compound-A, DS-1594, BMF-219, DSP-5336, the antibody A300-105A (commercially available from Bethyl Laboratories), MI-0202, MI-503, MI-463, MI-136, MI-3454, M-808 and ML-227, or a pharmaceutically acceptable salt thereof.

In some aspects, the present disclosure is directed to a method of treating, preventing, or reducing the severity of a colorectal cancer in a subject in need thereof comprising administering:
a pharmaceutical composition comprising a menin-MLL inhibitor at least once daily,
a pharmaceutical composition comprising a CYP3A4 inhibitor at least once daily, and wherein the subject is not also receiving an EGFR inhibitor, and wherein the menin-MLL inhibitor selected from the group consisting of Compound B, KO-539, JNJ-75276617,
Compound-A, DS-1594, BMF-219, DSP-5336, the antibody A300-105A (commercially available from Bethyl Laboratories), MI-0202, MI-503, MI-463, MI-136, MI-3454, M-808 and ML-227, or a pharmaceutically acceptable salt thereof.

In some aspects, the present disclosure is directed to a method of treating, preventing, or reducing the severity of a colorectal cancer in a subject in need thereof comprising administering:
a pharmaceutical composition comprising a menin-MLL inhibitor at least once daily,
a pharmaceutical composition comprising a CYP3A4 inhibitor at least once daily, and wherein the method further comprises administering a menin-MLL inhibitor in the absence of an EGFR inhibitor, and wherein the menin-MLL inhibitor selected from the group consisting of Compound B, KO-539, JNJ-75276617, Compound-A, DS-1594, BMF-219, DSP-5336, the antibody A300-105A (commercially available from Bethyl Laboratories), MI-0202, MI-503, MI-463, MI-136, MI-3454, M-808 and ML-227, or a pharmaceutically acceptable salt thereof.

In some aspects, the present disclosure is directed to a method of treating, preventing, or reducing the severity of a colorectal cancer in a subject in need thereof comprising administering:
a pharmaceutical composition comprising a menin-MLL inhibitor at least once daily,
a pharmaceutical composition comprising a CYP3A4 inhibitor at least once daily, and wherein the method further comprises administering the menin-MLL inhibitor in the absence of an EGFR inhibitor, and wherein the menin-MLL inhibitor selected from the group consisting of Compound B, KO-539, JNJ-75276617, Compound-A, DS-1594, BMF-219, DSP-5336, the antibody A300-105A (commercially available from Bethyl Laboratories), MI-0202, MI-503, MI-463, MI-136, MI-3454, M-808 and ML-227, or a pharmaceutically acceptable salt thereof.

In some aspects, the present disclosure is directed to a method of treating, preventing, or reducing the severity of a colorectal cancer in a subject in need thereof comprising administering:
a pharmaceutical composition comprising a menin-MLL inhibitor at least once daily, and
a pharmaceutical composition comprising cobicistat at least once daily.

In some aspects, the present disclosure is directed to a method of treating, preventing, or reducing the severity of a colorectal cancer in a subject in need thereof comprising administering:
a pharmaceutical composition comprising a menin-MLL inhibitor at least once daily,
a pharmaceutical composition comprising cobicistat at least once daily, and
wherein the method does not involve administering an EGFR inhibitor.

In some aspects, the present disclosure is directed to a method of treating, preventing, or reducing the severity of a colorectal cancer in a subject in need thereof comprising administering:
a pharmaceutical composition comprising a menin-MLL inhibitor at least once daily,
a pharmaceutical composition comprising cobicistat at least once daily, and
wherein the subject has not received an EGFR inhibitor.

In some aspects, the present disclosure is directed to a method of treating, preventing, or reducing the severity of a colorectal cancer in a subject in need thereof comprising administering:
a pharmaceutical composition comprising a menin-MLL inhibitor at least once daily,
a pharmaceutical composition comprising cobicistat at least once daily, and
wherein the subject is not also receiving an EGFR inhibitor.

In some aspects, the present disclosure is directed to a method of treating, preventing, or reducing the severity of a colorectal cancer in a subject in need thereof comprising administering:
a pharmaceutical composition comprising a menin-MLL inhibitor at least once daily,
a pharmaceutical composition comprising cobicistat at least once daily, and
wherein the method further comprises administering a menin-MLL inhibitor in the absence of an EGFR inhibitor.

In some aspects, the present disclosure is directed to a method of treating, preventing, or reducing the severity of a colorectal cancer in a subject in need thereof comprising administering:
a pharmaceutical composition comprising a menin-MLL inhibitor at least once daily,
a pharmaceutical composition comprising cobicistat at least once daily, and
wherein the method further comprises administering the menin-MLL inhibitor in the absence of an EGFR inhibitor.

In some aspects, the present disclosure is directed to a method of treating, preventing, or reducing the severity of a colorectal cancer in a subject in need thereof comprising administering:
a pharmaceutical composition comprising a menin-MLL inhibitor at least once daily, and
a pharmaceutical composition comprising cobicistat at least once daily, and wherein the menin-MLL inhibitor selected from the group consisting of Compound B, KO-539, JNJ-75276617, Compound-A, DS-1594, BMF-219, DSP-5336, the antibody A300-105A (commercially available from Bethyl Laboratories), MI-0202, MI-503, MI-463, MI-136, MI-3454, M-808 and ML-227, or a pharmaceutically acceptable salt thereof.

In some aspects, the present disclosure is directed to a method of treating, preventing, or reducing the severity of a colorectal cancer in a subject in need thereof comprising administering:
a pharmaceutical composition comprising a menin-MLL inhibitor at least once daily,
a pharmaceutical composition comprising cobicistat at least once daily, and wherein the method does not involve administering an EGFR inhibitor, and wherein the menin-MLL inhibitor selected from the group consisting of Compound B, KO-539, JNJ-75276617, Compound-A, DS-1594, BMF-219, DSP-5336, the antibody A300-105A (commercially available from Bethyl Laboratories), MI-0202, MI-503, MI-463, MI-136, MI-3454, M-808 and ML-227, or a pharmaceutically acceptable salt thereof.

In some aspects, the present disclosure is directed to a method of treating, preventing, or reducing the severity of a colorectal cancer in a subject in need thereof comprising administering:
a pharmaceutical composition comprising a menin-MLL inhibitor at least once daily,
a pharmaceutical composition comprising cobicistat at least once daily, and wherein the subject has not received an EGFR inhibitor, and wherein the menin-MLL inhibitor selected from the group consisting of Compound B, KO-539, JNJ-75276617, Compound-A, DS-1594, BMF-219, DSP-5336, the antibody A300-105A (commercially available from Bethyl Laboratories), MI-0202, MI-503, MI-463, MI-136, MI-3454, M-808 and ML-227, or a pharmaceutically acceptable salt thereof.

In some aspects, the present disclosure is directed to a method of treating, preventing, or reducing the severity of a colorectal cancer in a subject in need thereof comprising administering:
a pharmaceutical composition comprising a menin-MLL inhibitor at least once daily,
a pharmaceutical composition comprising cobicistat at least once daily, and wherein the subject is not also receiving an EGFR inhibitor, and wherein the menin-MLL inhibitor selected from the group consisting of Compound B, KO-539, JNJ-75276617, Compound-A, DS-1594, BMF-219, DSP-5336, the antibody A300-105A (commercially available from Bethyl Laboratories), MI-0202, MI-503, MI-463, MI-136, MI-3454, M-808 and ML-227, or a pharmaceutically acceptable salt thereof.

In some aspects, the present disclosure is directed to a method of treating, preventing, or reducing the severity of a colorectal cancer in a subject in need thereof comprising administering:
a pharmaceutical composition comprising a menin-MLL inhibitor at least once daily,
a pharmaceutical composition comprising cobicistat at least once daily, and wherein the method further comprises administering a menin-MLL inhibitor in the absence of an EGFR inhibitor, and wherein the menin-MLL inhibitor selected from the group consisting of Compound B, KO-539, JNJ-75276617, Compound-A, DS-1594, BMF-219, DSP-5336, the antibody A300-105A (commercially available from Bethyl Laboratories), MI-0202, MI-503, MI-463, MI-136, MI-3454, M-808 and ML-227, or a pharmaceutically acceptable salt thereof.

In some aspects, the present disclosure is directed to a method of treating, preventing, or reducing the severity of a colorectal cancer in a subject in need thereof comprising administering:
a pharmaceutical composition comprising a menin-MLL inhibitor at least once daily,
a pharmaceutical composition comprising cobicistat at least once daily, and wherein the method further comprises administering the menin-MLL inhibitor in the absence of an EGFR inhibitor, and wherein the menin-MLL inhibitor selected from the group consisting of Compound B, KO-539, JNJ-75276617, Compound-A, DS-1594, BMF-219, DSP-5336, the antibody A300-105A (commercially available from Bethyl Laboratories), MI-0202, MI-503, MI-463, MI-136, MI-3454, M-808 and ML-227, or a pharmaceutically acceptable salt thereof.

In some aspects, the present disclosure is directed to a method of treating, preventing, or reducing the severity of a colorectal cancer in a subject in need thereof comprising administering:
a pharmaceutical composition comprising Compound A at least once daily,
a pharmaceutical composition comprising cobicistat at least once daily, and
wherein the method further comprises administering Compound A in the absence of an EGFR inhibitor.

In some aspects, the present disclosure is directed to a method of treating, preventing, or reducing the severity of a colorectal cancer in a subject in need thereof comprising administering Compound A at a dose between about 150 mg and about 300 mg three times daily, wherein administering Compound A occurs in the absence of a CYP3A inhibitor and.or an EGFR inhibitor.

In some aspects, the present disclosure is directed to a method of treating, preventing, or reducing the severity of a colorectal cancer in a subject in need thereof comprising administering Compound A at a dose between about 150 mg and about 300 mg three times daily, wherein the subject does not receive a CYP3A inhibitor and/or an EGFR inhibitor.

In some embodiments, the colorectal cancer is characterized by dysregulation of the WNT/β-catenin signaling pathway.

In some embodiments, the subject has progressed on a prior colorectal cancer therapy due to developing resistance to the prior colorectal cancer therapy.

In some embodiments, the subject has relapsed from at least one prior colorectal cancer therapy due to developing resistance to the prior colorectal cancer therapy.

In some embodiments, the subject has relapsed from at least two prior colorectal cancer therapies due to developing resistance to the prior colorectal cancer therapies.

In some aspects, the present disclosure is directed to a method of treating, preventing, or reducing the severity of a cancer comprising administering a pharmaceutical composition comprising Compound A, wherein the method comprises administering Compound A three times per day between about 220 mg and about 280 mg, wherein a CYP3A inhibitor is not administered.

In some aspects, the present disclosure is directed to a method of treating, preventing, or reducing the severity of a cancer in a subject comprising administering a pharmaceutical composition comprising Compound A, wherein the method comprises administering Compound A three times per day between about 220 mg and about 280 mg, wherein the subject does not receive a CYP3A inhibitor.

In some embodiments, the cancer is a leukemia.

In some embodiments, the cancer is an AML leukemia.

In some embodiments, the cancer is an acute lymphoblastic leukemia.

In some embodiments, the cancer is an NMP1-mutated leukemia.

In some embodiments, the cancer is a colorectal cancer.

In some embodiments, the cancer is a microsatellite stable/proficient mismatch repair (MSS/pMMR) mCRC.

In some embodiments, the menin-MLL inhibitor is administered at a dosage of about 270 mg to about 280 mg.

In some embodiments, the menin-MLL inhibitor is administered three times per day.

In some embodiments, the menin-MLL inhibitor is administered with or without food.

In some aspects, the present disclosure is directed to a menin-MLL inhibitor for use in the preparation of a medicament for the treatment of colorectal cancer according to the method of any one of the previous claims, wherein the medicament and/or treatment does not involve the use of an EGFR inhibitor.

In some aspects, the present disclosure is directed to a menin-MLL inhibitor for use in the preparation of a medicament for the treatment of colorectal cancer according to the method of any one of the previous claims, wherein the medicament does not comprise an EGFR inhibitor.

In some aspects, the present disclosure is directed to a kit comprising a menin-MLL inhibitor, and printed instructions for using the menin-MLL inhibitor in a subject for the treatment for colorectal cancer.

In some aspects, the present disclosure is directed to a kit comprising a therapeutically effective amount of a menin-MLL inhibitor, and printed instructions for using the menin-MLL inhibitor in a subject for the treatment for colorectal cancer.

In some embodiments, the kit provides instructions for administering a menin-MLL inhibitor at a dosage of about 270 mg to about 280 mg.

In some embodiments, the kit provides instructions for administering a menin-MLL inhibitor at least once per day.

In some embodiments, the kit provides instructions for administering a menin-MLL inhibitor once per day.

In some embodiments, the kit provides instructions for administering a menin-MLL inhibitor twice per day.

In some embodiments, the kit provides instructions for administering a menin-MLL inhibitor three times per day.

In some embodiments, the kit provides instructions for administering about 276 mg of a menin-MLL inhibitor three times per day.

In some embodiments, the kit provides instructions for administering about 276 mg of a menin-MLL inhibitor three times per day with food.

In some embodiments, the kit provides instructions for administering about 276 mg of a menin-MLL inhibitor three times per day without food.

Unless otherwise defined, all technical and scientific terms used herein have the same meaning as commonly understood by one of ordinary skill in the art to which this disclosure belongs. The terminology used in the description is for describing particular embodiments only and is not intended to be limiting of the invention.

Where a range of values is provided, it is understood that each intervening value, to the tenth of the unit of the lower limit unless the context clearly dictates otherwise (such as in the case of a group containing a number of carbon atoms in which case each carbon atom number falling within the range is provided), between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either or both of those included limits are also included in the invention.

The articles “a” and “an” as used herein and in the appended claims are used herein to refer to one or to more than one (i.e., to at least one) of the grammatical object of the article unless the context clearly indicates otherwise. By way of example, “an element” means one element or more than one element.

### Colorectal Cancer

Colorectal cancer develops in the colon or in the rectum. If it starts in the colon, it may be referred to as colon cancer. If it starts in the rectum, it may be called rectal cancer. In some embodiments, the term “colorectal cancer” describes cancers of the colon. In some embodiments, the term “colorectal cancer” describes cancers of the rectum.

A common type of colorectal cancer is adenocarcinoma. Adenocarcinomas of the colon and rectum make up 95 percent of all colorectal cancer cases. In the gastrointestinal tract, rectal and colon adenocarcinomas develop in the cells of the lining inside the large intestine. These adenocarcinomas typically start as a growth of tissue called a polyp. A particular type of polyp called an adenoma may develop into cancer. Other types of tumors may develop in the colon or rectum and include: primary colorectal lymphomas, gastrointestinal stromal tumors, leiomyosarcomas, carcinoid tumors, and melanomas.

### Prior Treatments

LONSURF® is a combination of trifluridine, a nucleoside metabolic inhibitor, and tipiracil, a thymidine phosphorylase inhibitor, indicated for the treatment of adult subjects with metastatic colorectal cancer who have been previously treated with fluoropyrimidine-, oxaliplatin- and irinotecan-based chemotherapy, an anti-VEGF biological therapy, and if RAS wildtype, an anti-EGFR therapy; metastatic gastric or gastroesophageal junction adenocarcinoma previously treated with at least two prior lines of chemotherapy that included a fluoropyrimidine, a platinum, either a taxane or irinotecan, and if appropriate, HER2/neu-targeted therapy.

STIVARGA® (regorafenib) is a kinase inhibitor indicated for the treatment of subjects with metastatic colorectal cancer (CRC) who have been previously treated with fluoropyrimidine-, oxaliplatin- and irinotecan-based chemotherapy, an anti VEGF therapy, and, if KRAS wild type, an anti-EGFR therapy.

In some embodiments, the subject received at least one prior therapy. In some embodiments, the prior therapy is for treatment of cancer. In some embodiments, the prior therapy is for colon cancer. In some embodiments, the prior therapy is for rectal cancer. In some embodiments, the prior therapy is for colorectal cancer. In some embodiments, the prior therapy is for metastatic colorectal cancer. In some embodiments, the subject previously received LONSURF or STIVARGA. In some embodiments, the subject previously received LONSURF. In some embodiments, the subject previously received STIVARGA.

In some embodiments, the subject has progressed on a prior therapy. In some embodiments, the subject has progressed on at least one prior therapy. In some embodiments, the subject progressed on prior therapy with LONSURF or STIVARGA. In some embodiments, the subject progressed on prior therapy with LONSURF. In some embodiments, the subject progressed on prior therapy with STIVARGA.

In some embodiments, the subject has relapsed from prior line of therapy. In some embodiments, the subject has relapsed at least one prior therapy. In some embodiments, the subject relapsed from prior therapy with LONSURF or STIVARGA. In some embodiments, the subject relapsed from prior therapy with LONSURF. In some embodiments, the subject relapsed from prior therapy with STIVARGA.

In some embodiments, the subject has refractory cancer and had prior therapy. In some embodiments, the subject has refractory cancer and had at least one prior therapy. In some embodiments, the subject has refractory cancer and had prior therapy with LONSURF or STIVARGA. In some embodiments, the subject has refractory cancer and bad prior therapy with LONSURF. In some embodiments, the subject has refractory cancer and had prior therapy with STIVARGA.

In some embodiments, the subject progressed on prior therapy with one or more of fluoropyrimidine-, oxaliplatin- and irinotecan-based chemotherapy, an anti VEGF therapy, or an anti-EGFR therapy. In some embodiments, the subject progressed on prior therapy with fluoropyrimidine-based chemotherapy. In some embodiments, the subject progressed on prior therapy with one oxaliplatin-based chemotherapy. In some embodiments, the subject progressed on prior therapy with irinotecan-based chemotherapy. In some embodiments, the subject progressed on prior therapy with an anti VEGF therapy. In some embodiments, the subject progressed on prior therapy with an anti-EGFR therapy.

### Other Indications

In some aspects, the present disclosure is directed to a method of treating, preventing, or reducing the severity of a cancer comprising administering a pharmaceutical composition comprising Compound A, wherein the method comprises administering Compound A three times per day, and the subject does not receive a CYP3A inhibitor.

In some aspects, the present disclosure is directed to a method of treating, preventing, or reducing the severity of a cancer comprising administering a pharmaceutical composition comprising Compound A, wherein the method comprises administering Compound A three times per day, and the subject does not receive a CYP3A4 inhibitor.

In some aspects, the present disclosure is directed to a method of treating, preventing, or reducing the severity of a cancer comprising administering a pharmaceutical composition comprising Compound A, wherein the method comprises administering Compound A in the absence of a CYP3A/CYP3A4 inhibitor, wherein the dose of Compound A is administered three times per day.

In some embodiments, the cancer is a hematological cancer such as leukemia or lymphoma. Example leukemia and lymphomas treatable by the compounds of the invention include mixed lineage leukemia (MLL), MLL-related leukemia, MLL-associated leukemia, MLL-positive leukemia, MLL-induced leukemia, rearranged mixed lineage leukemia (MLL-r), leukemia associated with a MIL rearrangement or a rearrangement of the MLL gene, acute leukemia, chronic leukemia, indolent leukemia, lymphoblastic leukemia, lymphocytic leukemia, myeloid leukemia, myelogenous leukemia, childhood leukemia, acute lymphocytic leukemia (ALL) (also referred to as acute lymphoblastic leukemia or acute lymphoid leukemia), acute myeloid leukemia (AML) (also referred to as acute myelogenous leukemia or acute myeloblastic leukemia), acute granulocytic leukemia, acute nonlymphocytic leukemia, chronic lymphocytic leukemia (CLL) (also referred to as chronic lymphoblastic leukemia), chronic myelogenous leukemia (CML) (also referred to as chronic myeloid leukemia), therapy related leukemia, myelodysplastic syndrome (MDS), myeloproliferative disease (MPD) (such as primary myelofibrosis (PMF), myeloproliferative neoplasia (MPN), plasma cell neoplasm, multiple myeloma, myelodysplasia, cutaneous T-cell lymphoma, nucleophosmin (NPM1) AML, lymphoid neoplasm, AIDS-related lymphoma, thymoma, thymic carcinoma, mycosis fungoides, Alibert-Bazin syndrome, granuloma fungoides. Sezary Syndrome, hairy cell leukemia, T-cell prolymphocytic leukemia (T-PLL), large granular lymphocytic leukemia, meningeal leukemia, leukemic leptomeningitis, leukemic meningitis, multiple myeloma, Hodgkin's lymphoma, non Hodgkin's lymphoma (malignant lymphoma), and Waldenstrom's macroglobulinemia. In some embodiments, the acute myeloid leukemia (AML) is abstract nucleophosmin (NPM1)-mutated acute myeloid leukemia (i.e., NPM 1^{mut} acute myloid leukemia).

In some embodiments, the methods of the present disclosure are used to treat leukemia associated with a MLL rearrangement, acute lymphocytic leukemia associated with a MLL rearrangement, acute lymphoblastic leukemia associated with a MLL rearrangement, acute lymphoid leukemia associated with a MLL rearrangement, acute myeloid leukemia associated with a MLL rearrangement, acute myelogenous leukemia associated with a MLL rearrangement, or acute myeloblastic leukemia associated with a MLL rearrangement. As used herein, “MLL rearrangement” means a rearrangement of the MLL gene.

### Dosing

In some embodiments, the amount of the menin-MLL inhibitor that is administered is about 1 mg/day to about 1,000 mg/day. In some embodiments, the amount of the menin-MLL inhibitor that is administered is about 10 mg/day to about 1,000 mg/day. In some embodiments, the amount of the menin-MLL inhibitor that is administered is about 100 mg/day to about 1,000 mg/day. In some embodiments, the amount of the menin-MLL inhibitor that is administered is about 200 mg/day to about 1,000 mg/day. In some embodiments, the amount of the menin-MLL inhibitor that is administered is about 300 mg/day to about 1,000 mg/day. In some embodiments, the amount of the menin-MLL inhibitor that is administered is about 400 mg/day to about 1,000 mg/day. In some embodiments, the amount of the menin-MLL inhibitor that is administered is about 500 mg/day to about 1,000 mg/day. In some embodiments, the amount of the menin-MLL inhibitor that is administered is about 600 mg/day to about 1,000 mg/day. In some embodiments, the amount of the menin-MLL inhibitor that is administered is about 700 mg/day to about 1,000 mg/day. In some embodiments, the amount of the menin-MLL inhibitor that is administered is about 800 mg/day to about 1,000 mg/day.

In some embodiments, the amount of the menin-MLL inhibitor that is administered is about 1 mg/day to about 900 mg/day. In some embodiments, the amount of the menin-MLL inhibitor that is administered is about 10 mg/day to about 900 mg/day. In some embodiments, the amount of the menin-MLL inhibitor that is administered is about 100 mg/day to about 900 mg/day. In some embodiments, the amount of the menin-MLL inhibitor that is administered is about 200 mg/day to about 900 mg/day. In some embodiments, the amount of the menin-MLL inhibitor that is administered is about 300 mg/day to about 900 mg/day. In some embodiments, the amount of the menin-MLL inhibitor that is administered is about 400 mg/day to about 900 mg/day. In some embodiments, the amount of the menin-MLL inhibitor that is administered is about 500 mg/day to about 900 mg/day. In some embodiments, the amount of the menin-MLL inhibitor that is administered is about 600 mg/day to about 900 mg/day. In some embodiments, the amount of the menin-MLL inhibitor that is administered is about 700 mg/day to about 900 mg/day. In some embodiments, the amount of the menin-MLL inhibitor that is administered is about 800 mg/day to about 900 mg/day.

In some embodiments, the amount of the menin-MLL inhibitor that is administered is from about 25 mg/day to about 828 mg/day, from about 75 mg/day to about 828 mg/day, from about 100 mg/day to about 828 mg/day, from about 150 mg/day to about 828 mg/day, from about 200 mg/day to about 828 mg/day, from about 250 mg/day to about 828 mg/day, from about 300 mg/day to about 828 mg/day, from about 350 mg/day to about 828 mg/day, from about 400 mg/day to about 828 mg/day, from about 450 mg/day to about 828 mg/day, from about 500 mg/day to about 828 mg/day, from about 550 mg/day to about 828 mg/day, from about 600 mg/day to about 828 mg/day, from about 650 mg/day to about 828 mg/day, from about 700 mg/day to about 828 mg/day, or from about 750 mg/day to about 828 mg/day.

In some embodiments, the amount of Compound A that is administered is from about 25 mg/day to about 828 mg/day, from about 75 mg/day to about 828 mg/day, from about 100 mg/day to about 828 mg/day, from about 150 mg/day to about 828 mg/day, from about 200 mg/day to about 828 mg/day, from about 250 mg/day to about 828 mg/day, from about 300 mg/day to about 828 mg/day, from about 350 mg/day to about 828 mg/day, from about 400 mg/day to about 828 mg/day, from about 450 mg/day to about 828 mg/day, from about 500 mg/day to about 828 mg/day, from about 550 mg/day to about 828 mg/day, from about 600 mg/day to about 828 mg/day, from about 650 mg/day to about 828 mg/day, from about 700 mg/day to about 828 mg/day, or from about 750 mg/day to about 828 mg/day.

In some embodiments, the menin-MLL inhibitor is administered at a starting dosage, followed by escalation or de-escalation of the dosage.

In some embodiments, Compound A is administered at a starting dosage, followed by escalation or de-escalation of the dosage.

In some embodiments, the menin-MLL inhibitor is administered at a starting dosage of about 163 mg TID, followed by escalation to about 226 mg TID or de-escalation to 113 mg TID.

In some embodiments, Compound A is administered at a starting dosage of about 163 mg TID, followed by escalation to about 226 mg TID or de-escalation to 113 mg TID.

In some embodiments, the menin-MLL inhibitor is administered at a starting dosage of about 163 mg TID (489 mg total daily dose), followed by escalation or de-escalation according to the dose levels provided in Table A.

TABLE A
Dosing Schedule Using the Menin-MLL Inhibitor

| Dose Level | Dose |
|---|---|
| −3 | 25 mg TID |
| −2 | 50 mg TID |
| −1 | 113 mg TID |
| 1ª | 163 mg TID |
| 2 | 226 mg TID |
| 3 | 276 mg TID |

^{a}starting dose; TID = 3 times a day.

In some embodiments, Compound A is administered at a starting dosage of about 163 mg TID (489 mg total daily dose), followed by escalation or de-escalation according to the dose levels provided in Table B.

TABLE B
Dosing Schedule Using Compound A

| Dose Level | Dose |
|---|---|
| −3 | 25 mg TID |
| −2 | 50 mg TID |
| −1 | 113 mg TID |
| 1ª | 163 mg TID |
| 2 | 226 mg TID |
| 3 | 276 mg TID |

^{a}starting dose; TID = 3 times a day.

In some embodiments, the amount of Compound A that is administered is about 75 mg/day to about 330 mg/day. In some embodiments, the amount of Compound A that is administered is about 150 mg/day to about 330 mg/day.

In some embodiments, the amount of Compound A that is administered is about 75 mg/day to about 480 mg/day. In some embodiments, the amount of Compound A that is administered is about 150 mg/day to about 480 mg/day. In some embodiments, the amount of Compound A that is administered is about 330 mg/day to about 480 mg/day.

In some embodiments, the amount of Compound A that is administered is about 75 mg/day to about 670 mg/day. In some embodiments, the amount of Compound A that is administered is about 150 mg/day to about 670 mg/day. In some embodiments, the amount of Compound A that is administered is about 330 mg/day to about 670 mg/day. In some embodiments, the amount of Compound A that is administered is about 480 mg/day to about 670 mg/day

In some embodiments, the amount of Compound A that is administered is about 75 mg/day to about 850 mg/day. In some embodiments, the amount of Compound A that is administered is about 150 mg/day to about 850 mg/day. In some embodiments, the amount of Compound A that is administered is about 330 mg/day to about 850 mg/day. In some embodiments, the amount of Compound A that is administered is about 480 mg/day to about 850 mg/day. In some embodiments, the amount of Compound A that is administered is about 670 mg/day to about 850 mg/day.

In some embodiments, the daily dosage is divided into multiple administrations and is given once a day, given twice a day, given three times per day, or given four times per day. In some embodiments, the menin-MLL inhibitor is administered once per day, twice per day, three times per day. In some embodiments, the menin-MLL inhibitor daily dosage is divided into multiple administrations and is given once per day. In some embodiments, the menin-MLL inhibitor daily dosage is divided into multiple administrations and is given twice per day. In some embodiments, the menin-MLL inhibitor daily dosage is divided into multiple administrations and is given three times per day.

In some embodiments, the menin-MIL inhibitor is administered at about 50 mg QD (once a day), about 75 mg QD, about 100 mg QD, about 113 mg QD, about 125 mg QD, about 150 mg QD, about 175 mg QD, about 200 mg QD, about 50 mg ql2 h, about 75 mg ql2 h, about 100 mg ql2 h, about 113 mg ql2 h, about 125 mg ql2 h, about 150 mg ql2 h, about 163 mg ql2 h, about 175 mg ql2 h, about 200 mg ql2 h, about 225 mg ql2 h, about 226 mg ql2 h, about 250 mg ql2 h, about 275 mg ql2 h, about 300 mg ql2 h, about 325 mg ql2 h, about 339 mg ql2 h, about 350 mg ql2 h, about 375 mg ql2 h, about 400 mg ql2 h, about 425 mg ql2 h, about 450 mg ql2 h, about 452 mg ql2 h, about 475 mg ql2b, about 500 mg ql2 h, about 525 mg ql2 h, about 550 mg ql2 h, about 565 mg ql2 h, about 575 mg ql2 h, or about 600 mg ql2 h. In some embodiments, the menin-MLL inhibitor is Compound A and is administered at about 50 mg QD, about 75 mg QD, about 100 mg QD, about 113 mg QD, about 125 mg QD, about 150 mg QD, about 175 mg QD, about 200 mg QD, about 50 mg ql2 h, about 75 mg ql2 h, about 100 mg ql2 h, about 113 mg ql2 h, about 125 mg ql2 h, about 150 mg ql2 h, about 163 mg ql2 h, about 175 mg ql2 h, about 200 mg ql2 h, about 225 mg ql2 h, about 226 mg ql2 h. about 250 mg ql2 h, about 275 mg ql2 h, about 300 mg ql2 h, about 325 mg ql2 h, about 339 mg ql2 h, about 350 mg ql2 h, about 375 mg ql2 h, about 400 mg ql2 h, about 425 mg ql2 h, about 450 mg ql2 h, about 452 mg ql2 h, about 475 mg ql2 h, about 500 mg ql2 h, about 525 mg ql2 h, about 550 mg ql2 h, about 565 mg ql2 h, about 575 mg ql2 h, or about 600 mg ql2 h. In some embodiments, the menin-MLL inhibitor is a pharmaceutical formulation comprising Compound A and is administered at about 50 mg QD, about 75 mg QD, about 100 mg QD, about 113 mg QD, about 125 mg QD, about 150 mg QD, about 175 mg QD, about 200 mg QD, about 50 mg ql2 h, about 75 mg ql2 h, about 100 mg ql2b, about 113 mg ql2 h, about 125 mg ql2 h, about 150 mg ql2 h, about 163 mg ql2 h, about 175 mg ql2 h, about 200 mg 912 h, about 225 mg ql2 h, about 226 mg ql2 h, about 250 mg ql2 h, about 275 mg ql2 h, about 300 mg ql2 h, about 325 mg ql2 h, about 339 mg ql2 h, about 350 mg ql2 h, about 375 mg 912 h, about 400 mg ql2 h, about 425 mg ql2 h, about 450 mg ql2 h, about 452 mg ql2 h, about 475 mg ql2 h, about 500 mg ql2 h, about 525 mg ql2 h, about 550 mg ql2 h, about 565 mg ql2 h, about 575 mg ql2 h, or about 600 mg ql2 h. In some embodiments, menin-MLL inhibitor is a capsule comprising Compound A and is administered at about 50 mg QD, about 75 mg QD, about 100 mg QD, about 113 mg QD, about 125 mg QD, about 150 mg QD, about 175 mg QD, about 200 mg QD, about 50 mg ql2 h, about 75 mg ql2 h, about 100 mg ql2 h, about 113 mg ql2 h, about 125 mg ql2 h, about 150 mg ql2 h, about 163 mg ql2 h, about 175 mg ql2 h, about 200 mg ql2 h, about 225 mg ql2 h, about 226 mg ql2b, about 250 mg ql2 h, about 275 mg ql2 h, about 300 mg ql2 h, about 325 mg ql2 h, about 339 mg ql2 h, about 350 mg 912 h. about 375 mg ql2 h, about 400 mg ql2 h, about 425 mg ql2 h, about 450 mg ql2 h, about 452 mg ql2 h, about 475 mg ql2 h, about 500 mg ql2 h, about 525 mg ql2 h, about 550 mg ql2 h, about 565 mg ql2 h, about 575 mg ql2 h, or about 600 mg ql2 h

In some embodiments, the menin-MLL inhibitor is a pharmaceutical formulation comprising Compound B and is administered at about 163 mg ql2 h. In some embodiments, menin-MLL inhibitor is a capsule comprising Compound B and is administered at about 163 mg q12 h.

In some embodiments, the menin-MLL inhibitor is administered at a dose of about 25 mg TID, about 50 mg TID, about 75 mg TID, about 100 mg TID, about 113 mg TID, about 125 mg TID, about 150 mg TID, about 163 mg TID, about 175 mg TID, about 200 mg TID, about 225 mg TID, about 226 mg TID, about 250 mg TID, about 275 mg TID, about 276 mg TID, or about 300 mg TID. In some embodiments, the menin-MLL inhibitor is Compound A and is administered at a dose of about 25 mg TID, about 50 mg TID, about 75 mg TID, about 100 mg TID, about 113 mg TID, about 125 mg TID, about 150 mg TID, about 163 mg TID, about 175 mg TID, about 200 mg TID, about 225 mg TID, about 226 mg TID, about 250 mg TID, about 275 mg TID, about 276 mg TID, or about 300 mg TID. In some embodiments, the menin-MLL inhibitor is within a pharmaceutical formulation comprising Compound A and is administered at a dose of about 25 mg TID, about 50 mg TID, about 75 mg TID, about 100 mg TID, about 113 mg TID, about 125 mg TID, about 150 mg TID, about 163 mg TID, about 175 mg TID, about 200 mg TID, about 225 mg TID, about 226 mg TID, about 250 mg TID, about 275 mg TID, about 276 mg TID, or about 300 mg TID. In some embodiments, menin-MLL inhibitor is a capsule comprising Compound A and is administered at a dose of about 25 mg TID, about 50 mg TID, about 75 mg TID, about 100 mg TID, about 113 mg TID, about 125 mg TID, about 150 mg TID, about 163 mg TID, about 175 mg TID, about 200 mg TID, about 225 mg TID, about 226 mg TID, about 250 mg TID, about 275 mg TID, about 276 mg TID, or about 300 mg TID

In some embodiments, the menin-MLL inhibitor is administered in the absence of a CYP3A inhibitor at a dose of about 25 mg TID, about 50 mg TID, about 75 mg TID, about 100 mg TID, about 113 mg TID, about 125 mg TID, about 150 mg TID, about 163 mg TID, about 175 mg TID, about 200 mg TID, about 225 mg TID, about 226 mg TID, about 250 mg TID, about 275 mg TID, about 276 mg TID, or about 300 mg TID. In some embodiments, the menin-MLL inhibitor is Compound A and is administered in the absence of a CYP3A inhibitor at a dose of about 25 mg TID, about 50 mg TID, about 75 mg TID, about 100 mg TID, about 113 mg TID, about 125 mg TID, about 150 mg TID, about 163 mg TID, about 175 mg TID, about 200 mg TID, about 225 mg TID, about 226 mg TID, about 250 mg TID, about 275 mg TID, about 276 mg TID, or about 300 mg TID. In some embodiments, the menin-MLL inhibitor is within a pharmaceutical formulation comprising Compound A and is administered in the absence of a CYP3A inhibitor at a dose of about 25 mg TID, about 50 mg TID, about 75 mg TID, about 100 mg TID, about 113 mg TID, about 125 mg TID, about 150 mg TID, about 163 mg TID, about 175 mg TID, about 200 mg TID, about 225 mg TID, about 226 mg TID, about 250 mg TID, about 275 mg TID, about 276 mg TID, or about 300 mg TID. In some embodiments, menin-MLL inhibitor is a capsule comprising Compound A and is administered in the absence of a CYP3A inhibitor at a dose of about 25 mg TID, about 50 mg TID, about 75 mg TID, about 100 mg TID, about 113 mg TID, about 125 mg TID, about 150 mg TID, about 163 mg TID, about 175 mg TID, about 200 mg TID, about 225 mg TID, about 226 mg TID, about 250 mg TID, about 275 mg TID, about 276 mg TID, or about 300 mg TID.

In some embodiments, the menin-MLL inhibitor is administered at a dose of about 163 mg every 12 hours. In some embodiments, the menin-MLL inhibitor is Compound B and is administered at a dose of about 163 mg every 12 hours. In some embodiments, the menin-MLL inhibitor is within a pharmaceutical formulation comprising Compound B and is administered at a dose of about 163 mg every 12 hours. In some embodiments, menin-MLL inhibitor is a capsule comprising Compound B and is administered at a dose of about 163 mg every 12 hours.

In some embodiments, the menin-MLL inhibitor is administered in the absence of a CYP3A inhibitor at a dose of about 163 mg every 12 hours. In some embodiments, the menin-MLL inhibitor is Compound B and is administered in the absence of a CYP3A inhibitor at a dose of about 163 mg every 12 hours. In some embodiments, the menin-MLL inhibitor is within a pharmaceutical formulation comprising Compound B and is administered in the absence of a CYP3A inhibitor at a dose of about 163 mg every 12 hours. In some embodiments, menin-MLL inhibitor is a capsule comprising Compound B and is administered in the absence of a CYP3A inhibitor at a dose of about 163 mg every 12 hours.

In some embodiments, doses are taken no less than 6 hours after the previous dose.

In some embodiments, the method of treating, preventing, or reducing the severity of a colorectal cancer in a subject in need thereof, comprises administering to the subject a menin-MLL inhibitor or a pharmaceutical composition comprising a menin-MLL inhibitor, wherein the menin-MLL inhibitor is administered at a dosage of between about 25 mg and about 276 mg every 12 hours, and wherein upon administration, the area under plasma concentration versus time curve from time 0 to 24 hours (AUC₍₀₋₂₄₎) of compound A is from about 1,000 ng*h/mL to about 200,000 ng*h/mL, from about 1,000 ng*h/mL to about 150,000 ng*h/mL, from about 1,000 ng*h/mL to about 100,000 ng*h/mL, from about 1,000 ng*h/mL to about 50,000 ng*h/mL, from about 1,000 ng*h/mL to about 40,000 ng*h/mL, from about 1,000 ng*h/mL to about 30,000 ng*h/mL, from about 1,000 ng*h/mL to about 20,000 ng*h/mL, from about 1,000 ng*h/mL to about 10,000 ng*h/mL, from about 10,000 ng*h/mL to about 200,000 ng*h/mL, from about 20,000 ng*h/mL to about 200,000 ng*h/mL, from about 30,000 ng*h/mL to about 200,000 ng*h/mL, from about 40,000 ng*h/mL to about 200,000 ng*h/mL, from about 50,000 ng*h/mL to about 200,000 ng*h/mL, from about 60,000 ng*h/mL to about 200,000 ng*h/mL, from about 70,000 ng*h/mL to about 200,000 ng*h/mL, from about 80,000 ng*h/mL to about 200,000 ng*h/mL, from about 90,000 ng*h/mL to about 200,000 ng*h/mL, from about 100,000 ng*h/mL to about 200,000 ng*h/mL, from about 10,000 ng*h/mL to about 50,000 ng*h/mL, from about 10,000 ng*h/mL to about 45,000 ng*h/mL, from about 10,000 ng*h/mL to about 40,000 ng*h/mL, from about 10,000 ng*h/mL to about 35,000 ng*h/mL, from about 10,000 ng*h/mL to about 30,000 ng*h/mL, from about 10,000 ng*h/mL to about 25,000 ng*h/mL, from about 10,000 ng*h/mL to about 20,000 ng*h/mL, or from about 10,000 ng*h/mL to about 15,000 ng*h/mL.

In some embodiments, the method of treating, preventing, or reducing the severity of a colorectal cancer in a subject in need thereof, comprising administering to the subject a menin-MLL inhibitor or a pharmaceutical composition comprising a menin-MLL inhibitor, wherein the menin-MLL inhibitor is administered at a dosage of up to about 828 mg per day, and wherein upon administration, the area under plasma concentration versus time curve from time 0 to 24 hours (AUC₍₀₋₂₄₎) of compound A is from about 1,000 ng*h/mL to about 200,000 ng*h/mL, from about 1,000 ng*h/mL to about 150,000 ng*h/mL, from about 1,000 ng*h/mL to about 100,000 ng*h/mL, from about 1,000 ng*h/mL to about 50,000 ng*h/mL, from about 1,000 ng*h/mL to about 40,000 ng*h/mL, from about 1,000 ng*h/mL to about 30,000 ng*h/mL, from about 1,000 ng*h/mL to about 20,000 ng*h/mL, from about 1,000 ng*h/mL to about 10,000 ng*h/mL, from about 10,000 ng*h/mL to about 200,000 ng*h/mL, from about 20,000 ng*h/mL to about 200,000 ng*h/mL, from about 30,000 ng*h/mL to about 200,000 ng*h/mL, from about 40,000 ng*h/mL to about 200,000 ng*h/mL, from about 50,000 ng*h/mL to about 200,000 ng*h/mL, from about 60,000 ng*h/mL to about 200,000 ng*h/mL, from about 70,000 ng*h/mL to about 200,000 ng*h/mL, from about 80,000 ng*h/mL to about 200,000 ng*h/mL, from about 90,000 ng*h/mL to about 200,000 ng*h/mL, from about 100,000 ng*h/mL to about 200,000 ng*h/mL, from about 10,000 ng*h/mL to about 50,000 ng*h/mL, from about 10,000 ng*h/mL to about 45,000 ng*h/mL, from about 10,000 ng*h/mL to about 40,000 ng*h/mL, from about 10,000 ng*h/mL to about 35,000 ng*h/mL, from about 10,000 ng*h/mL to about 30,000 ng*h/mL, from about 10,000 ng*h/mL to about 25,000 ng*h/mL, from about 10,000 ng*h/mL to about 20,000 ng*h/mL, or from about 10,000 ng*h/mL to about 15,000 ng*h/mL.

In some embodiments, the method of treating, preventing, or reducing the severity of a colorectal cancer in a subject in need thereof, comprises administering to the subject a menin-MLL inhibitor or a pharmaceutical composition comprising a menin-MLL inhibitor and a CYP3A4 inhibitor, wherein the menin-MLL inhibitor is administered at a dosage of between about 25 mg and about 276 mg every 12 hours, and wherein upon administration, the area under plasma concentration versus time curve from time 0 to 24 hours (AUC₍₀₋₂₄₎) of compound A is from about 1,000 ng*h/mL to about 200,000 ng*h/mL, from about 1,000 ng*h/mL to about 150,000 ng*h/mL, from about 1,000 ng*h/mL to about 100,000 ng*h/mL, from about 1,000 ng*h/mL to about 50,000 ng*h/mL, from about 1,000 ng*h/mL to about 40,000 ng*h/mL, from about 1,000 ng*h/mL to about 30,000 ng*h/mL, from about 1,000 ng*h/mL to about 20,000 ng*h/mL, from about 1,000 ng*h/mL to about 10,000 ng*h/mL, from about 10,000 ng*h/mL to about 200,000 ng*h/mL, from about 20,000 ng*h/mL to about 200,000 ng*h/mL, from about 30,000 ng*h/mL to about 200,000 ng*h/mL, from about 40,000 ng*h/mL to about 200,000 ng*h/mL, from about 50,000 ng*h/mL to about 200,000 ng*h/mL, from about 60,000 ng*h/mL to about 200,000 ng*h/mL, from about 70,000 ng*h/mL to about 200,000 ng*h/mL, from about 80,000 ng*h/mL to about 200,000 ng*h/mL, from about 90,000 ng*h/mL to about 200,000 ng*h/mL, from about 100,000 ng*h/mL to about 200,000 ng*h/mL, from about 10,000 ng*h/mL to about 50,000 ng*h/mL, from about 10,000 ng*h/mL to about 45,000 ng*h/mL, from about 10,000 ng*h/mL to about 40,000 ng*h/mL, from about 10,000 ng*h/mL to about 35,000 ng*h/mL, from about 10,000 ng*h/mL to about 30,000 ng*h/mL, from about 10,000 ng*h/mL to about 25,000 ng*h/mL, from about 10,000 ng*h/mL to about 20,000 ng*h/mL, or from about 10,000 ng*h/mL to about 15,000 ng*h/mL.

In some embodiments, the method of treating, preventing, or reducing the severity of a colorectal cancer in a subject in need thereof, comprising administering to the subject a menin-MLL inhibitor or a pharmaceutical composition comprising a menin-MLL inhibitor and a CYP3A4 inhibitor, wherein the menin-MLL inhibitor is administered at a dosage of up to about 828 mg per day, and wherein upon administration, the area under plasma concentration versus time curve from time 0 to 24 hours (AUC₍₀₋₂₄₎) of compound A is from about 1,000 ng*h/mL to about 200,000 ng*h/mL, from about 1,000 ng*h/mL to about 150,000 ng*h/mL, from about 1,000 ng*h/mL to about 100,000 ng*h/mL, from about 1,000 ng*h/mL to about 50,000 ng*h/mL, from about 1,000 ng*h/mL to about 40,000 ng*h/mL, from about 1,000 ng*h/mL to about 30,000 ng*h/mL, from about 1,000 ng*h/mL to about 20,000 ng*h/mL, from about 1,000 ng*h/mL to about 10,000 ng*h/mL, from about 10,000 ng*h/mL to about 200,000 ng*h/mL, from about 20,000 ng*h/mL to about 200,000 ng*h/mL, from about 30,000 ng*h/mL to about 200,000 ng*h/mL, from about 40,000 ng*h/mL to about 200,000 ng*h/mL, from about 50,000 ng*h/mL to about 200,000 ng*h/mL, from about 60,000 ng*h/mL to about 200,000 ng*h/mL, from about 70,000 ng*h/mL to about 200,000 ng*h/mL, from about 80,000 ng*h/mL to about 200,000 ng*h/mL, from about 90,000 ng*h/mL to about 200,000 ng*h/mL, from about 100,000 ng*h/mL to about 200,000 ng*h/mL, from about 10,000 ng*h/mL to about 50,000 ng*h/mL, from about 10,000 ng*h/mL to about 45,000 ng*h/mL, from about 10,000 ng*h/mL to about 40,000 ng*h/mL, from about 10,000 ng*h/mL to about 35,000 ng*h/mL, from about 10,000 ng*h/mL to about 30,000 ng*h/mL, from about 10,000 ng*h/mL to about 25,000 ng*h/mL, from about 10,000 ng*h/mL to about 20,000 ng*h/mL, or from about 10,000 ng*h/mL to about 15,000 ng*h/mL.

In some embodiments, the method of treating, preventing, or reducing the severity of a colorectal cancer in a subject in need thereof, comprises administering to the subject a menin-MLL inhibitor or a pharmaceutical composition comprising a menin-MLL inhibitor, wherein the menin-MLL inhibitor is administered at a dosage of between about 25 mg and about 276 mg every 12 hours, and wherein upon administration, the minimum observed concentration (Cₘᵢₙ) of compound A is from about 100 ng/ml to about 5,000 ng/ml, from about 100 ng/ml to about 4,000 ng/mL, from about 100 ng/mL to about 3,000 ng/ml, from about 100 ng/ml to about 2,000 ng/mL, from about 100 ng/mL to about 2,000 ng/mL, from about 100 ng/ml to about 1,000 ng/mL, from about 100 ng/ml to about 900 ng/mL, from about 100 ng/ml to about 800 ng/mL, from about 100 ng/mL to about 700 ng/mL, from about 100 ng/ml to about 600 ng/ml, from about 100 ng/mL to about 500 ng/ml, from about 100 ng/mL to about 400 ng/mL, from about 100 ng/mL to about 300 ng/ml, from about 100 ng/mL to about 200 ng/mL, from about 200 ng/ml to about 2,000 ng/ml, from about 200 ng/ml to about 1,000 ng/mL, from about 300,000 ng/mL to about 1,000 ng/ml, from about 300 ng/mL to about 1,000 ng/mL, from about 400 ng/ml to about 1,000 ng/ml, from about 500 ng/ml to about 1,000 ng/mL, from about 600 ng/ml to about 1,000 ng/ml, from about 700 ng/ml to about 1,000 ng/mL, from about 800 ng/ml to about 1,000 ng/ml, from about 900 ng/mL to about 1,000 ng/mL.

In some embodiments, the method of treating, preventing, or reducing the severity of a colorectal cancer in a subject in need thereof, comprising administering to the subject a menin-MLL inhibitor or a pharmaceutical composition comprising a menin-MLL inhibitor, wherein the menin-MLL inhibitor is administered at a dosage of up to about 828 mg per day, and wherein upon administration, the minimum observed concentration (Cₘᵢₙ) of compound A is from about 100 ng/ml to about 5,000 ng/mL, from about 100 ng/mL to about 4,000 ng/mL, from about 100 ng/mL to about 3,000 ng/mL, from about 100 ng/ml to about 2,000 ng/ml, from about 100 ng/mL to about 2,000 ng/mL, from about 100 ng/ml to about 1,000 ng/ml, from about 100 ng/mL to about 900 ng/mL, from about 100 ng/ml to about 800 ng/mL, from about 100 ng/mL to about 700 ng/mL, from about 100 ng/mL to about 600 ng/mL, from about 100 ng/mL to about 500 ng/ml, from about 100 ng/ml to about 400 ng/mL, from about 100 ng/mL to about 300 ng/ml, from about 100 ng/mL to about 200 ng/ml, from about 200 ng/mL to about 2,000 ng/mL, from about 200 ng/ml to about 1,000 ng/mL, from about 300,000 ng/mL to about 1,000 ng/mL, from about 300 ng/mL to about 1,000 ng/mL, from about 400 ng/mL to about 1,000 ng/mL, from about 500 ng/ml to about 1,000 ng/mL, from about 600 ng/mL to about 1,000 ng/mL, from about 700 ng/ml to about 1,000 ng/mL, from about 800 ng/ml to about 1,000 ng/mL, from about 900 ng/ml to about 1,000 ng/mL.

In some embodiments, the method of treating, preventing, or reducing the severity of a colorectal cancer in a subject in need thereof, comprises administering to the subject a menin-MLL inhibitor or a pharmaceutical composition comprising a menin-MLL inhibitor and a CYP3A4 inhibitor, wherein the menin-MLL inhibitor is administered at a dosage of between about 25 mg and about 276 mg every 12 hours, and wherein upon administration, the minimum observed concentration (Cₘᵢₙ) of compound A is from about 100 ng/ml to about 5,000 ng/mL, from about 100 ng/mL to about 4,000 ng/mL, from about 100 ng/ml to about 3,000 ng/mL, from about 100 ng/mL to about 2,000 ng/mL, from about 100 ng/ml to about 2,000 ng/ml, from about 100 ng/mL to about 1,000 ng/mL, from about 100 ng/ml to about 900 ng/mL, from about 100 ng/ml to about 800 ng/mL, from about 100 ng/ml to about 700 ng/ml, from about 100 ng/ml to about 600 ng/ml, from about 100 ng/ml to about 500 ng/mL, from about 100 ng/mL to about 400 ng/ml, from about 100 ng/ml to about 300 ng/ml, from about 100 ng/ml to about 200 ng/ml, from about 200 ng/ml to about 2,000 ng/mL, from about 200 ng/mL to about 1,000 ng/mL, from about 300,000 ng/ml to about 1,000 ng/mL, from about 300 ng/mL to about 1,000 ng/mL, from about 400 ng/ml to about 1,000 ng/mL, from about 500 ng/mL to about 1,000 ng/ml, from about 600 ng/ml to about 1,000 ng/mL, from about 700 ng/ml to about 1,000 ng/mL, from about 800 ng/ml to about 1,000 ng/mL, from about 900 ng/ml to about 1,000 ng/mL.

In some embodiments, the method of treating, preventing, or reducing the severity of a colorectal cancer in a subject in need thereof, comprising administering to the subject a menin-MLL inhibitor or a pharmaceutical composition comprising a menin-MLL inhibitor and a CYP3A4 inhibitor, wherein the menin-MLL inhibitor is administered at a dosage of up to about 828 mg per day, and wherein upon administration, the minimum observed concentration (Cₘᵢₙ) of compound A is from about 100 ng/ml to about 5,000 ng/ml, from about 100 ng/mL to about 4,000 ng/mL, from about 100 ng/ml to about 3,000 ng/mL, from about 100 ng/ml to about 2,000 ng/mL, from about 100 ng/ml to about 2,000 ng/ml, from about 100 ng/ml to about 1,000 ng/ml, from about 100 ng/ml to about 900 ng/ml, from about 100 ng/mL to about 800 ng/mL, from about 100 ng/ml to about 700 ng/ml, from about 100 ng/ml to about 600 ng/ml, from about 100 ng/ml to about 500 ng/ml, from about 100 ng/ml to about 400 ng/mL, from about 100 ng/ml to about 300 ng/mL, from about 100 ng/ml to about 200 ng/mL, from about 200 ng/ml to about 2,000 ng/ml, from about 200 ng/mL to about 1,000 ng/mL, from about 300,000 ng/mL to about 1,000 ng/ml, from about 300 ng/mL to about 1,000 ng/mL, from about 400 ng/ml to about 1,000 ng/ml, from about 500 ng/ml to about 1,000 ng/mL, from about 600 ng/ml to about 1,000 ng/ml, from about 700 ng/ml to about 1,000 ng/mL, from about 800 ng/mL to about 1,000 ng/ml, from about 900 ng/ml to about 1,000 ng/mL.

In some embodiments, the method of treating, preventing, or reducing the severity of a colorectal cancer in a subject in need thereof, comprises administering to the subject a menin-MLL inhibitor or a pharmaceutical composition comprising a menin-MLL inhibitor, wherein the menin-MLL inhibitor is administered at a dosage of between about 25 mg and about 276 mg every 12 hours, and wherein upon administration, the maximum observed concentration (Cₘₐₓ) of compound A is from about 1,000 ng/mL to about 200,000 ng/ml, from about 1,000 ng/mL to about 150,000 ng/mL, from about 1,000 ng/mL to about 100,000 ng/mL, from about 1,000 ng/mL to about 50,000 ng/mL, from about 1,000 ng/mL to about 40,000 ng/mL, from about 1,000 ng/mL to about 30,000 ng/mL, from about 1,000 ng/mL to about 20,000 ng/mL, from about 1,000 ng/mL to about 10,000 ng/mL, from about 10,000 ng/mL to about 200,000 ng/mL, from about 20,000 ng/mL to about 200,000 ng/mL, from about 30,000 ng/mL to about 200,000 ng/mL, from about 40,000 ng/mL to about 200,000 ng/mL, from about 50,000 ng/mL to about 200,000 ng/mL, from about 60,000 ng/ml to about 200,000 ng/mL, from about 70,000 ng/mL to about 200,000 ng/mL, from about 80,000 ng/ml to about 200,000 ng/mL, from about 90,000 ng/mL to about 200,000 ng/mL, from about 100,000 ng/mL to about 200,000 ng/mL, from about 10,000 ng/mL to about 50,000 ng/mL, from about 10,000 ng/mL to about 45,000 ng/mL, from about 10,000 ng/mL to about 40,000 ng/mL, from about 10,000 ng/mL to about 35,000 ng/mL, from about 10,000 ng/ml to about 30,000 ng/mL, from about 10,000 ng/mL to about 25,000 ng/mL, from about 10,000 ng/mL to about 20,000 ng/mL, or from about 10,000 ng/mL to about 15,000 ng/mL.

In some embodiments, the method of treating, preventing, or reducing the severity of a colorectal cancer in a subject in need thereof, comprising administering to the subject a menin-MLL inhibitor or a pharmaceutical composition comprising a menin-MLL inhibitor, wherein the menin-MLL inhibitor is administered at a dosage of up to about 828 mg per day, and wherein upon administration, the maximum observed concentration (Cₘₐₓ) of compound A is from about 1,000 ng/ml to about 200,000 ng/mL, from about 1,000 ng/mL to about 150,000 ng/mL, from about 1,000 ng/mL to about 100,000 ng/mL, from about 1,000 ng/ml to about 50,000 ng/mL, from about 1,000 ng/mL to about 40,000 ng/mL, from about 1,000 ng/mL to about 30,000 ng/mL, from about 1,000 ng/mL to about 20,000 ng/mL, from about 1,000 ng/mL to about 10,000 ng/mL, from about 10,000 ng/ml to about 200,000 ng/mL, from about 20,000 ng/mL to about 200,000 ng/mL, from about 30,000 ng/mL to about 200,000 ng/mL, from about 40,000 ng/mL to about 200,000 ng/mL, from about 50,000 ng/mL to about 200,000 ng/mL, from about 60,000 ng/mL to about 200,000 ng/mL, from about 70,000 ng/mL to about 200,000 ng/mL, from about 80,000 ng/mL to about 200,000 ng/mL, from about 90,000 ng/mL to about 200,000 ng/mL, from about 100,000 ng/ml to about 200,000 ng/mL, from about 10,000 ng/mL to about 50,000 ng/mL, from about 10,000 ng/ml to about 45,000 ng/mL, from about 10,000 ng/mL to about 40,000 ng/mL, from about 10,000 ng/mL to about 35,000 ng/mL, from about 10,000 ng/mL to about 30,000 ng/ml, from about 10,000 ng/mL to about 25,000 ng/mL, from about 10,000 ng/mL to about 20,000 ng/mL, or from about 10,000 ng/ml to about 15,000 ng/mL.

In some embodiments, the method of treating, preventing, or reducing the severity of a colorectal cancer in a subject in need thereof, comprises administering to the subject a menin-MLL inhibitor or a pharmaceutical composition comprising a menin-MLL inhibitor and a CYP3A4 inhibitor, wherein the menin-MLL inhibitor is administered at a dosage of between about 25 mg and about 276 mg every 12 hours, and wherein upon administration, the maximum observed concentration (Cₘₐₓ) of compound A is from about 1,000 ng/mL to about 200,000 ng/mL, from about 1,000 ng/mL to about 150,000 ng/mL, from about 1,000 ng/ml to about 100,000 ng/mL, from about 1,000 ng/mL to about 50,000 ng/mL, from about 1,000 ng/mL to about 40,000 ng/mL, from about 1,000 ng/mL to about 30,000 ng/mL, from about 1,000 ng/mL to about 20,000 ng/mL, from about 1,000 ng/mL to about 10,000 ng/mL, from about 10,000 ng/mL to about 200,000 ng/mL, from about 20,000 ng/mL to about 200,000 ng/mL, from about 30,000 ng/mL to about 200,000 ng/mL, from about 40,000 ng/mL to about 200,000 ng/mL, from about 50,000 ng/mL to about 200,000 ng/ml, from about 60,000 ng/mL to about 200,000 ng/mL, from about 70,000 ng/mL to about 200,000 ng/mL, from about 80,000 ng/mL to about 200,000 ng/mL, from about 90,000 ng/ml to about 200,000 ng/mL, from about 100,000 ng/mL to about 200,000 ng/mL, from about 10,000 ng/mL to about 50,000 ng/mL, from about 10,000 ng/mL to about 45,000 ng/ml, from about 10,000 ng/mL to about 40,000 ng/mL, from about 10,000 ng/mL to about 35,000 ng/mL, from about 10,000 ng/mL to about 30,000 ng/mL, from about 10,000 ng/mL to about 25,000 ng/mL, from about 10,000 ng/mL to about 20,000 ng/mL, or from about 10,000 ng/ml to about 15,000 ng/mL.

In some embodiments, the method of treating, preventing, or reducing the severity of a colorectal cancer in a subject in need thereof, comprising administering to the subject a menin-MLL inhibitor or a pharmaceutical composition comprising a menin-MLL inhibitor and a CYP3A4 inhibitor, wherein the menin-MLL inhibitor is administered at a dosage of up to about 828 mg per day, and wherein upon administration, the maximum observed concentration (Cₘₐₓ) of compound A is from about 1,000 ng/mL to about 200,000 ng/ml, from about 1,000 ng/mL to about 150,000 ng/mL, from about 1,000 ng/ml to about 100,000 ng/mL, from about 1,000 ng/mL to about 50,000 ng/mL, from about 1,000 ng/mL to about 40,000 ng/mL, from about 1,000 ng/mL to about 30,000 ng/mL, from about 1,000 ng/ml to about 20,000 ng/mL, from about 1,000 ng/mL to about 10,000 ng/mL, from about 10,000 ng/mL to about 200,000 ng/mL, from about 20,000 ng/mL to about 200,000 ng/mL, from about 30,000 ng/mL to about 200,000 ng/mL, from about 40,000 ng/mL to about 200,000 ng/mL, from about 50,000 ng/mL to about 200,000 ng/mL, from about 60,000 ng/mL to about 200,000 ng/mL, from about 70,000 ng/mL to about 200,000 ng/mL, from about 80,000 ng/ml to about 200,000 ng/mL, from about 90,000 ng/mL to about 200,000 ng/mL, from about 100,000 ng/mL to about 200,000 ng/mL, from about 10,000 ng/mL to about 50,000 ng/mL, from about 10,000 ng/mL to about 45,000 ng/mL, from about 10,000 ng/ml to about 40,000 ng/mL, from about 10,000 ng/mL to about 35,000 ng/mL, from about 10,000 ng/mL to about 30,000 ng/mL, from about 10,000 ng/mL to about 25,000 ng/mL, from about 10,000 ng/ml to about 20,000 ng/mL, or from about 10,000 ng/ml to about 15,000 ng/mL.

In some embodiments, the present disclosure is directed to a method of administering Compound A without a CPY3A inhibitor (e.g. a CYP3A4 inhibitor) yet achieving comparable Cₘₐₓ, Cₘᵢₙ, and AUC achieved by administering with a CPY3A inhibitor (e.g. a CYP3A4 inhibitor). In some embodiments, this method improves patient compliance by reducing the number medications administered.

In some embodiments, the present disclosure is directed to a method of treating, preventing, or reducing the severity of a cancer in a subject in need thereof, comprises administering to the subject Compound A, or a pharmaceutically acceptable salt thereof, or a pharmaceutical composition comprising Compound A, wherein Compound A is administered at a dosage of between about 163 mg and about 276 mg every 8 hours, wherein upon administration, the area under plasma concentration versus time curve from time 0 to 24 hours (AUC₍₀₋₂₄₎) of Compound A is from about 1,000 ng*h/mL to about 100,000 ng*h/mL.

In some embodiments, the present disclosure is directed to a method of treating, preventing, or reducing the severity of a cancer in a subject in need thereof, comprises administering to the subject Compound A, or a pharmaceutically acceptable salt thereof, or a pharmaceutical composition comprising Compound A, wherein Compound A, or the pharmaceutically acceptable salt thereof, or the pharmaceutical composition comprising Compound A is administered at a dosage of about 163 mg every 8 hours, wherein upon administration the area under plasma concentration versus time curve from time 0 to 24 hours (AUC₍₀₋₂₄₎) of Compound A is from about 10,000 ng*h/mL to about 60,000 ng*h/mL.

In some embodiments, the present disclosure is directed to a method of treating, preventing, or reducing the severity of a cancer in a subject in need thereof, comprises administering to the subject Compound A, or a pharmaceutically acceptable salt thereof, or a pharmaceutical composition comprising Compound A, wherein Compound A, or the pharmaceutically acceptable salt thereof, or the pharmaceutical composition comprising Compound A is administered at a dosage of about 226 mg every 8 hours, wherein upon administration the area under plasma concentration versus time curve from time 0 to 24 hours (AUC₍₀₋₂₄₎) of Compound A is from about 14,000 ng*h/mL to about 65,000 ng*h/mL.

In some embodiments, the present disclosure is directed to a method of treating, preventing, or reducing the severity of a cancer in a subject in need thereof, comprises administering to the subject Compound A, or a pharmaceutically acceptable salt thereof, or a pharmaceutical composition comprising Compound A, wherein Compound A, or the pharmaceutically acceptable salt thereof, or the pharmaceutical composition comprising Compound A is administered at a dosage of about 276 mg every 8 hours, wherein upon administration the area under plasma concentration versus time curve from time 0 to 24 hours (AUC₍₀₋₂₄₎) of Compound A is from about 17,000 ng*h/mL to about 90,000 ng*h/mL.

In some embodiments, the present disclosure is directed to a method of treating, preventing, or reducing the severity of a cancer in a subject in need thereof, comprises administering to the subject Compound A, or a pharmaceutically acceptable salt thereof, or a pharmaceutical composition comprising Compound A, wherein Compound A, or the pharmaceutically acceptable salt thereof, or the pharmaceutical composition comprising Compound A is administered at a dosage of about 163 mg every 8 hours, wherein upon administration the area under plasma concentration versus time curve from time 0 to 24 hours (AUC₍₀₋₂₄₎) of Compound A is about 22,000 ng*h/mL.

In some embodiments, the present disclosure is directed to a method of treating, preventing, or reducing the severity of a cancer in a subject in need thereof, comprises administering to the subject Compound A, or a pharmaceutically acceptable salt thereof, or a pharmaceutical composition comprising Compound A, wherein Compound A, or the pharmaceutically acceptable salt thereof, or the pharmaceutical composition comprising

Compound A is administered at a dosage of about 226 mg every 8 hours, wherein upon administration the area under plasma concentration versus time curve from time 0 to 24 hours (AUC₍₀₋₂₄₎) of Compound A is about 30,500 ng*h/mL.

In some embodiments, the present disclosure is directed to a method of treating, preventing, or reducing the severity of a cancer in a subject in need thereof, comprises administering to the subject Compound A, or a pharmaceutically acceptable salt thereof, or a pharmaceutical composition comprising Compound A, wherein Compound A, or the pharmaceutically acceptable salt thereof, or the pharmaceutical composition comprising Compound A is administered at a dosage of about 276 mg every 8 hours, wherein upon administration the area under plasma concentration versus time curve from time 0 to 24 hours (AUC₍₀₋₂₄₎) of Compound A is about 41,000 ng*h/mL.

In some embodiments, the present disclosure is directed to a method of treating, preventing, or reducing the severity of a cancer in a subject in need thereof, comprises administering to the subject Compound A, or a pharmaceutically acceptable salt thereof, or a pharmaceutical composition comprising Compound A, wherein Compound A is administered at a dosage from about 163 mg to about 276 mg every 8 hours, and wherein upon administration the minimum observed concentration (Cₘᵢₙ) of Compound A is from about 100 ng/ml to about 2,400 ng/mL.

In some embodiments, the present disclosure is directed to a method of treating, preventing, or reducing the severity of a cancer in a subject in need thereof, comprises administering to the subject Compound A, or a pharmaceutically acceptable salt thereof, or a pharmaceutical composition comprising Compound A, wherein Compound A, or the pharmaceutically acceptable salt thereof, or the pharmaceutical composition comprising Compound A is administered at a dosage of about 163 mg every 8 hours, wherein upon administration the minimum observed concentration (Cₘᵢₙ) of Compound A is from about 100 ng/ml to about 1,400 ng/mL.

In some embodiments, the present disclosure is directed to a method of treating, preventing, or reducing the severity of a cancer in a subject in need thereof, comprises administering to the subject Compound A, or a pharmaceutically acceptable salt thereof, or a pharmaceutical composition comprising Compound A, wherein Compound A, or the pharmaceutically acceptable salt thereof, or the pharmaceutical composition comprising Compound A is administered at a dosage of about 226 mg every 8 hours, wherein upon administration the minimum observed concentration (Cₘᵢₙ) of Compound A is from about 100 ng/mL to about 1,600 ng/mL.

In some embodiments, the present disclosure is directed to a method of treating, preventing, or reducing the severity of a cancer in a subject in need thereof, comprises administering to the subject Compound A, or a pharmaceutically acceptable salt thereof, or a pharmaceutical composition comprising Compound A, wherein Compound A, or the pharmaceutically acceptable salt thereof, or the pharmaceutical composition comprising Compound A is administered at a dosage of about 276 mg every 8 hours, wherein upon administration the minimum observed concentration (Cₘᵢₙ) of Compound A is from about 100 ng/ml to about 2,400 ng/mL.

In some embodiments, the present disclosure is directed to a method of treating, preventing, or reducing the severity of a cancer in a subject in need thereof, comprises administering to the subject Compound A, or a pharmaceutically acceptable salt thereof, or a pharmaceutical composition comprising Compound A, wherein Compound A, or the pharmaceutically acceptable salt thereof, or the pharmaceutical composition comprising Compound A is administered at a dosage of about 163 mg every 8 hours, wherein upon administration the minimum observed concentration (Cₘᵢₙ) of Compound A is about 400 ng/mL.

In some embodiments, the present disclosure is directed to a method of treating, preventing, or reducing the severity of a cancer in a subject in need thereof, comprises administering to the subject Compound A, or a pharmaceutically acceptable salt thereof, or a pharmaceutical composition comprising Compound A, wherein Compound A, or the pharmaceutically acceptable salt thereof, or the pharmaceutical composition comprising Compound A is administered at a dosage of about 226 mg every 8 hours, wherein upon administration the minimum observed concentration (Cₘᵢₙ) of Compound A is about 500 ng/mL.

In some embodiments, the present disclosure is directed to a method of treating, preventing, or reducing the severity of a cancer in a subject in need thereof, comprises administering to the subject Compound A, or a pharmaceutically acceptable salt thereof, or a pharmaceutical composition comprising Compound A, wherein Compound A, or the pharmaceutically acceptable salt thereof, or the pharmaceutical composition comprising

Compound A is administered at a dosage of about 276 mg every 8 hours, wherein upon administration the minimum observed concentration (Cₘᵢₙ) of Compound A is about 800 ng/mL.

In some embodiments, the present disclosure is directed to a method of treating, preventing, or reducing the severity of a cancer in a subject in need thereof, comprises administering to the subject Compound A, or a pharmaceutically acceptable salt thereof, or a pharmaceutical composition comprising Compound A, wherein Compound A is administered at a dosage from about 163 mg to about 276 mg every 8 hours, and wherein upon administration the maximum observed concentration (Cₘₐₓ) of Compound A is from about 1,000 ng/mL to about 6,000 ng/mL.

In some embodiments, the present disclosure is directed to a method of treating, preventing, or reducing the severity of a cancer in a subject in need thereof, comprises administering to the subject Compound A, or a pharmaceutically acceptable salt thereof, or a pharmaceutical composition comprising Compound A, wherein Compound A, or the pharmaceutically acceptable salt thereof, or the pharmaceutical composition comprising Compound A is administered at a dosage of about 163 mg every 8 hours, wherein upon administration the maximum observed concentration (Cₘₐₓ) of Compound A is from about 1,000 ng/mL to about 3,400 ng/mL.

In some embodiments, the present disclosure is directed to a method of treating, preventing, or reducing the severity of a cancer in a subject in need thereof, comprises administering to the subject Compound A, or a pharmaceutically acceptable salt thereof, or a pharmaceutical composition comprising Compound A, wherein Compound A, or the pharmaceutically acceptable salt thereof, or the pharmaceutical composition comprising Compound A is administered at a dosage of about 226 mg every 8 hours, wherein upon administration the maximum observed concentration (Cₘₐₓ) of Compound A is from about 1,300 ng/mL to about 4,400 ng/mL.

In some embodiments, the present disclosure is directed to a method of treating, preventing, or reducing the severity of a cancer in a subject in need thereof, comprises administering to the subject Compound A, or a pharmaceutically acceptable salt thereof, or a pharmaceutical composition comprising Compound A, wherein Compound A, or the pharmaceutically acceptable salt thereof, or the pharmaceutical composition comprising Compound A is administered at a dosage of about 276 mg every 8 hours, wherein upon administration the maximum observed concentration (Cₘₐₓ) of Compound A is from about 1,700 ng/mL to about 5,400 ng/mL.

In some embodiments, the present disclosure is directed to a method of treating, preventing, or reducing the severity of a cancer in a subject in need thereof, comprises administering to the subject Compound A, or a pharmaceutically acceptable salt thereof, or a pharmaceutical composition comprising Compound A, wherein Compound A, or the pharmaceutically acceptable salt thereof, or the pharmaceutical composition comprising Compound A is administered at a dosage of about 163 mg every 8 hours, wherein upon administration maximum observed concentration (Cₘₐₓ) of Compound A is about 1700 ng/mL.

In some embodiments, the present disclosure is directed to a method of treating, preventing, or reducing the severity of a cancer in a subject in need thereof, comprises administering to the subject Compound A, or a pharmaceutically acceptable salt thereof, or a pharmaceutical composition comprising Compound A, wherein Compound A, or the pharmaceutically acceptable salt thereof, or the pharmaceutical composition comprising Compound A is administered at a dosage of about 226 mg every 8 hours, wherein upon administration the maximum observed concentration (Cₘₐₓ) of Compound A is about 2400 ng/mL.

In some embodiments, the present disclosure is directed to a method of treating, preventing, or reducing the severity of a cancer in a subject in need thereof, comprises administering to the subject Compound A, or a pharmaceutically acceptable salt thereof, or a pharmaceutical composition comprising Compound A, wherein Compound A, or the pharmaceutically acceptable salt thereof, or the pharmaceutical composition comprising Compound A is administered at a dosage of about 276 mg every 8 hours, wherein upon administration the maximum observed concentration (Cₘₐₓ) of Compound A is about 3,100 ng/mL.

In some embodiments, the present disclosure is directed to a method of treating, preventing, or reducing the severity of a cancer in a subject in need thereof, comprises administering to the subject Compound A, or a pharmaceutically acceptable salt thereof, or a pharmaceutical composition comprising Compound A and a CYP3A4 inhibitor, wherein Compound A is administered at a dosage of about 163 mg every 12 hours, and wherein upon administration the area under plasma concentration versus time curve from time 0 to 24 hours (AUC₍₀₋₂₄₎) of Compound A is from about 19,000 ng*h/mL to about 110,000 ng*h/mL.

In some embodiments, the present disclosure is directed to a method of treating, preventing, or reducing the severity of a cancer in a subject in need thereof, comprises administering to the subject Compound A, or a pharmaceutically acceptable salt thereof, or a pharmaceutical composition comprising Compound A and a CYP3A4 inhibitor, wherein Compound A is administered at a dosage of about 163 mg every 12 hours, and wherein upon administration the area under plasma concentration versus time curve from time 0 to 24 hours (AUC₍₀₋₂₄₎) of Compound A is about 43,000 ng*h/mL.

In some embodiments, the present disclosure is directed to a method of treating, preventing, or reducing the severity of a cancer in a subject in need thereof, comprises administering to the subject Compound A, or a pharmaceutically acceptable salt thereof, or a pharmaceutical composition comprising Compound A and a CYP3A4 inhibitor, wherein Compound A is administered at a dosage of about 163 mg every 12 hours, and wherein upon administration the minimum observed concentration (Cₘᵢₙ) of Compound A is from about 200 ng/ml to about 2,800 ng/mL.

In some embodiments, the present disclosure is directed to a method of treating, preventing, or reducing the severity of a cancer in a subject in need thereof, comprises administering to the subject Compound A, or a pharmaceutically acceptable salt thereof, or a pharmaceutical composition comprising Compound A and a CYP3A4 inhibitor, wherein Compound A is administered at a dosage of about 163 mg every 12 hours, and wherein upon administration the minimum observed concentration (Cₘᵢₙ) of Compound A is about 900 ng/mL.

In some embodiments, the present disclosure is directed to a method of treating, preventing, or reducing the severity of a cancer in a subject in need thereof, comprises administering to the subject Compound A, or a pharmaceutically acceptable salt thereof, or a pharmaceutical composition comprising Compound A and a CYP3A4 inhibitor, wherein Compound A is administered at a dosage of about 163 mg every 12 hours, and wherein upon administration the maximum observed concentration (Cₘₐₓ) of Compound A is from about 1,700 ng/mL to about 6,200 ng/mL.

In some embodiments, the present disclosure is directed to a method of treating, preventing, or reducing the severity of a cancer in a subject in need thereof, comprises administering to the subject Compound A, or a pharmaceutically acceptable salt thereof, or a pharmaceutical composition comprising Compound A and a CYP3A4 inhibitor, wherein Compound A is administered at a dosage of about 163 mg every 12 hours, and wherein upon administration the maximum observed concentration (Cₘₐₓ) of Compound A is about 3,200 ng/mL.

In some aspects, the present disclosure is directed to a method of treating, preventing, or reducing the severity of a colorectal cancer in a subject in need thereof comprising administering:
a pharmaceutical composition comprising a menin-MLL inhibitor at least once daily, and
a pharmaceutical composition comprising a CYP3A4 inhibitor at least once daily, wherein the subject has at least one of the following characteristics:
(a) a bone marrow reserve defined by: absolute neutrophil count (ANC)≥1,500/μL, platelet count ≥100,000/μL, and hemoglobin ≥9 g/dL;
(b) hepatic function defined by: total bilirubin≤1.5× the upper limit of normal (ULN); alanine aminotransferase (ALT) or aspartate aminotransferase (AST)≤2.5×ULN (≤5×ULN if the elevation can be reasonably ascribed to the presence of metastatic disease in liver);
(c) renal function defined by: estimated glomerular filtration rate ≥50 mL/min based on the Cockcroft-Gault formula for adults;
(d) meet a coagulation functional threshold defined by an international normalized ratio (INR) or prothrombin time (PT)≤1.5×ULN, unless the patient is receiving anticoagulant therapy, as long as PT or partial thromboplastin time (PTT) is within therapeutic range of intended use of anticoagulants;
(e) an activated PTT (aPTT)≤1.5×ULN, unless the patient is receiving anticoagulant therapy, as long as PT or PTT is within therapeutic range of intended use of anticoagulants; and
(f) potassium maintained at ≥4.0 mEq/L and magnesium at ≥2.0 mg/dL.

In some aspects, the present disclosure is directed to a method of treating, preventing, or reducing the severity of a colorectal cancer in a subject in need thereof comprising administering:
a pharmaceutical composition comprising Compound A at least once daily, and
a pharmaceutical composition comprising a CYP3A4 inhibitor at least once daily, wherein the subject has at least one of the following characteristics:
(a) a bone marrow reserve defined by: absolute neutrophil count (ANC)≥1,500/μL, platelet count ≥100,000/μL, and hemoglobin ≥9 g/dL;
(b) hepatic function defined by: total bilirubin≤1.5× the upper limit of normal (ULN); alanine aminotransferase (ALT) or aspartate aminotransferase (AST)≤2.5×ULN (≤5×ULN if the elevation can be reasonably ascribed to the presence of metastatic disease in liver);
(c) renal function defined by: estimated glomerular filtration rate ≥50 mL/min based on the Cockcroft-Gault formula for adults;
(d) meet a coagulation functional threshold defined by an international normalized ratio (INR) or prothrombin time (PT)≤1.5×ULN, unless the patient is receiving anticoagulant therapy, as long as PT or partial thromboplastin time (PTT) is within therapeutic range of intended use of anticoagulants;
(e) an activated PTT (aPTT)≤1.5×ULN, unless the patient is receiving anticoagulant therapy, as long as PT or PTT is within therapeutic range of intended use of anticoagulants; and
(f) potassium maintained at ≥4.0 mEq/L and magnesium at ≥2.0 mg/dL.

In some embodiments, the administration reduces the tumor volume in the subject by at least about 10%, at least about 20%, at least about 30%, at least about 40%, at least about 50%, at least about 60%, at least about 70%, at least about 80%, at least about 90%, at least about 95%, at least about 98%, or at least about 99%.

In some embodiments, dosing of the menin-MLL inhibitors that contemplated for use in the methods of the present disclosure are as known in the art at least e.g., WO 2017/112768, WO 2017/214367, WO 2018/053267, WO 2020/069027, WO 2021/207335, US 2021/0115018, US 2019/0307750, US 2016/0339035, and Borkin et al. Cancer cell vol. 27, 4 (2015): 589-602, the contents of each of which are incorporated by reference.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the disclosure is dictated by and directly dependent on the unique characteristics of the active compound and the particular therapeutic effect to be achieved.

It is to be understood that the pharmaceutical compositions can be included in a container, pack, or dispenser together with instructions for administration.

It is to be understood that, for the compounds of the present disclosure being capable of further forming salts, all of these forms are also contemplated within the scope of the claimed disclosure.

Techniques for formulation and administration of the disclosed compounds of the disclosure can be found in Remington: the Science and Practice of Pharmacy, 19^{th} edition, Mack Publishing Co., Easton, PA (1995). In an embodiment, the compounds described herein, and the pharmaceutically acceptable salts thereof, are used in pharmaceutical preparations in combination with a pharmaceutically acceptable carrier or diluent. Suitable pharmaceutically acceptable carriers include inert solid fillers or diluents and sterile aqueous or organic solutions. The compounds will be present in such pharmaceutical compositions in amounts sufficient to provide the desired dosage amount in the range described herein.

It is to be understood that, unless otherwise stated, any description of a method of treatment includes use of the compounds to provide such treatment or prophylaxis as is described herein, as well as use of the compounds to prepare a medicament to treat or prevent such condition. The treatment includes treatment of human or non-human animals including rodents and other disease models.

It is to be understood that, throughout the description, where compositions are described as having, including, or comprising specific components, it is contemplated that compositions also consist essentially of, or consist of, the recited components. Similarly, where methods or processes are described as having, including, or comprising specific process steps, the processes also consist essentially of, or consist of, the recited processing steps. Further, it should be understood that the order of steps or order for performing certain actions is immaterial so long as the invention remains operable. Moreover, two or more steps or actions can be conducted simultaneously.

### Definitions

As used herein, the term “subject” includes human and non-human animals, as well as cell lines, cell cultures, tissues, and organs. In some embodiments, the subject is a mammal. The mammal can be e.g., a human or appropriate non-human mammal, such as primate, mouse, rat, dog, cat, cow, horse, goat, camel, sheep or a pig. The subject can also be a bird or fowl. In some embodiments, the subject is a human.

As used herein, the term “subject in need thereof” refers to a subject having a disease or having an increased risk of developing the disease. A subject in need thereof can be one who has been previously diagnosed or identified as having a disease or disorder disclosed herein. A subject in need thereof can also be one who is suffering from a disease or disorder disclosed herein. Alternatively, a subject in need thereof can be one who has an increased risk of developing such disease or disorder relative to the population at large (i.e., a subject who is predisposed to developing such disorder relative to the population at large). A subject in need thereof can have a refractory or resistant a disease or disorder disclosed herein (i.e., a disease or disorder disclosed herein that does not respond or has not yet responded to treatment). The subject may be resistant at start of treatment or may become resistant during treatment. In some embodiments, the subject in need thereof received and failed all known effective therapies for a disease or disorder disclosed herein. In some embodiments, the subject in need thereof received at least one prior therapy.

As used herein, the term “treating” or “treat” describes the management and care of a subject for the purpose of combating a disease, condition, or disorder and includes the administration of a compound of the present disclosure, or a pharmaceutically acceptable salt, polymorph or solvate thereof, to alleviate the symptoms or complications of a disease, condition or disorder, or to eliminate the disease, condition or disorder. The term “treat” can also include treatment of a cell in vitro or an animal model.

As used herein, the term “preventing,” “prevent,” or “protecting against” describes reducing or eliminating the onset of the symptoms or complications of such disease, condition or disorder.

As used herein, the term “pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. A “pharmaceutically acceptable excipient” as used in the specification and claims includes both one and more than one such excipient.

As used herein, the term “therapeutically effective amount”, refers to an amount of a pharmaceutical agent to treat, ameliorate, or prevent an identified disease or condition, or to exhibit a detectable therapeutic or inhibitory effect. The effect can be detected by any assay method known in the art. The precise effective amount for a subject will depend upon the subject's body weight, size, and health; the nature and extent of the condition; and the therapeutic or combination of therapeutics selected for administration. Therapeutically effective amounts for a given situation can be determined by routine experimentation that is within the skill and judgment of the clinician.

All percentages and ratios used herein, unless otherwise indicated, are by weight. Other features and advantages of the present disclosure are apparent from the different examples. The provided examples illustrate different components and methodology useful in practicing the present disclosure. The examples do not limit the claimed disclosure. Based on the present disclosure the skilled artisan can identify and employ other components and methodology useful for practicing the present disclosure.

As used herein, the term “pharmaceutical composition” is a formulation containing the compounds of the present disclosure in a form suitable for administration to a subject. In one embodiment, the pharmaceutical composition is in bulk or in unit dosage form. The unit dosage form is any of a variety of forms, including, for example, a capsule, an IV bag, a tablet, a single pump on an aerosol inhaler or a vial. The quantity of active ingredient (e.g., a formulation of the disclosed compound or salt, hydrate, solvate or isomer thereof) in a unit dose of composition is an effective amount and is varied according to the particular treatment involved. One skilled in the art will appreciate that it is sometimes necessary to make routine variations to the dosage depending on the age and condition of the patient. The dosage will also depend on the route of administration. A variety of routes are contemplated, including oral, pulmonary, rectal, parenteral, transdermal, subcutaneous, intravenous, intramuscular, intraperitoneal, inhalational, buccal, sublingual, intrapleural, intrathecal, intranasal, and the like. Dosage forms for the topical or transdermal administration of a compound of this disclosure include powders, sprays, ointments, pastes, creams, lotions, gels, solutions, patches and inhalants. In one embodiment, the active compound is mixed under sterile conditions with a pharmaceutically acceptable carrier, and with any preservatives, buffers, or propellants that are required.

As used herein, the term “pharmaceutically acceptable” refers to those compounds, anions, cations, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

It is to be understood that a compound or pharmaceutical composition of the disclosure can be administered to a subject in many of the well-known methods currently used for chemotherapeutic treatment. For example, a compound of the disclosure may be injected into the blood stream or body cavities or taken orally or applied through the skin with patches. The dose chosen should be sufficient to constitute effective treatment but not so high as to cause unacceptable side effects. The state of the disease condition (e.g., a disease or disorder disclosed herein) and the health of the patient should preferably be closely monitored during and for a reasonable period after treatment.

Dosage and administration are adjusted to provide sufficient levels of the active agent(s) or to maintain the desired effect. Factors which may be taken into account include the severity of the disease state, general health of the subject, age, weight, and gender of the subject, diet, time and frequency of administration, drug combination(s), reaction sensitivities, and tolerance/response to therapy.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

The term “about” and the like, as used herein, in association with numeric values or ranges, reflects the fact that there is a certain level of variation that is recognized and tolerated in the art due to practical and/or theoretical limitations. For example, minor variation is tolerated due to inherent variances in the manner in which certain devices operate and/or measurements are taken. In accordance with the above, the phrase “about” is normally used to encompass values within the standard deviation or standard error.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

As used herein in the specification and in the claims, the phrase “at least one,” in reference to a list of one or more elements, should be understood to mean at least one element selected from anyone or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a nonlimiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, in certain methods described herein that include more than one step or act, the order of the steps or acts of the method is not necessarily limited to the order in which the steps or acts of the method are recited unless the context indicates otherwise.

All publications and patent documents cited herein are incorporated herein by reference as if each such publication or document was specifically and individually indicated to be incorporated herein by reference. Citation of publications and patent documents is not intended as an admission that any is pertinent prior art, nor does it constitute any admission as to the contents or date of the same. The invention having now been described by way of written description, those of skill in the art will recognize that the invention can be practiced in a variety of embodiments and that the foregoing description and examples below are for purposes of illustration and not limitation of the claims that follow.

### EXAMPLES

The following examples provide those of ordinary skill in the art with a complete disclosure and description of how to make and use the present invention and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be assumed. Standard abbreviations may be used, e.g. according to those described in Table 2, below.

TABLE 2
Abbreviations

| Abbreviation | Definition |
|---|---|
| AE | Adverse event |
| AESI | Adverse event of special interest |
| ANC | Absolute neutrophil count |
| AUC | Area under the plasma concentration versus time curve |
| AUC_{0-inf} | Area under the plasma concentration versus |
| | time curve from time 0 extrapolated to infinity |
| AUC₀₋₂₄ | Area under the plasma concentration versus |
| | time curve from time 0 to 24 hours |
| AUC₀₋ₜ | Area under the plasma concentration versus time |
| | curve from time 0 to the last measurable concentration |
| CL/F | Apparent oral clearance |
| Cₘₐₓ | Maximum observed concentration |
| Cₘᵢₙ | Minimum observed concentration |
| CRA | Clinical research associate |
| CRC | Colorectal cancer |
| CSR | Clinical study report |
| CT | Computed topography |
| ctDNA | Circulating tumor DNA |
| CTCAE | Common Terminology Criteria for Adverse Events |
| CxDy | Cycle x Day y |
| CYP | Cytochrome P450 |
| DCR₆ | Disease control rate at 6 cycles |
| DLT | Dose-limiting toxicity |
| ECG | Electrocardiogram |
| ECOG | Eastern Cooperative Oncology Group |
| EGFR | Epidermal growth factor receptor |
| EOT | End of Treatment |
| FDG | 18F-deoxyglucose |
| FSH | Follicle stimulating hormone |
| GCP | Good Clinical Practice |
| GI | Gastrointestinal |
| HBV | Hepatitis B virus |
| HCV | Hepatitis C virus |
| HRT | Hormonal replacement therapy |
| ITT | Intent-to-treat |
| IV | Intravenous |
| KMT2A | Lysine (K) methyltransferase 2A gene |
| mCRC | Metastatic colorectal cancer |
| MRI | Magnetic resonance imaging |
| ms | Millisecond(s) |
| MTD | Maximum tolerated dose |
| ORR | Overall response rate |
| OS | Overall survival |
| PD | Progressive disease |
| PDX | Patient-derived xenograft |
| PET | Positron emission tomography |
| PFS | Progression-free survival |
| PK | Pharmacokinetic |
| pMMR | Proficient mismatch repair |
| PO | Oral(ly) |
| PR | Partial remission |
| PT | Prothrombin time |
| PTT | Partial thromboplastin time |
| q12h | Every 12 hours |
| QTc | Corrected QT interval |
| QTcF | Corrected QT interval by Fridericia |
| R/R | Relapsed/refractory |
| RECIST | Response Evaluation Criteria in Solid Tumors |
| RP2D | Recommended Phase 2 dose |
| SAE | Serious adverse event |
| SAP | Statistical analysis plan |
| t1/2 | Apparent terminal elimination half-life |
| TEAE | Treatment-emergent adverse event |
| TID | Three times a day |
| Tₘₐₓ | Time to maximum plasma concentration |
| TRAE | Treatment-related adverse event |
| ULN | Upper limit of normal |
| Vz/F | Apparent volume of distribution during the terminal phase |

### Example 1. Treatment of Colorectal Cancer with Compound A

### Study Design:

This Phase 1/2 study evaluates Compound A in patients with colorectal cancer (CRC) who have failed at least one prior line of therapy. It comprises a Compound A dose-escalation using a Rolling 6 design (Phase 1a) and signal-seeking portion (Phase 1b), and a 2:1 (Compound A: Trifluridine/Tipiracil (LONSURF®) or Compound A: Regorafenib (STIVARGA®)) randomized signal-confirmation portion (Phase 2).

Patients enrolled into Phase 1 and the Compound A arm of Phase 2 receive Compound A, 3 times a day (TID) with food, continually in 28-day cycles. Patients enrolled into the Investigator Choice arm of the Phase 2 portion of the study receive either Trifluridine/Tipiracil (LONSURF®) or Regorafenib (STIVARGA®) at the respective drug label's dose and schedule.

### Rationale for Study Design

The starting dose level and subsequent dose levels are evaluated using a Rolling 6 dose evaluation model (Phase 1a). The dose identified as safe and tolerable is evaluated in a small number of patients for indications of anti-tumor activity (Phase 1b), prior to initiating a randomized evaluation of Compound A against either LONSURF® or STIVARGA® (Phase 2). The 2-step evaluation of anti-tumor activity, Phase 1b and Phase 2, is an approach intended to limit exposure of Compound A to patients if there are no early signs of anti-tumor activity.

### Phase 1

### Dose Escalation (Phase 1a)

The dose-escalation portion of the study employs a Rolling 6 trial design. Initially, up to 3 dose levels are being evaluated during dose escalation.

Up to six patients are enrolled into a dose cohort. Two to six patients can be concurrently enrolled into the cohort, dependent upon (1) the number of patients already enrolled in that cohort; (2) the number of patients who have experienced dose-limiting toxicity (DLT) at the dose being evaluated in that cohort; and (3) the number of patients entered but with tolerability data pending in that cohort. Accrual is suspended when a cohort of 6 has enrolled or when the study endpoints have been met.

For example, when three participants are enrolled into a dose cohort, if toxicity data are available for all three when the fourth participant enters and there are no DLTs, the fourth participant is enrolled at the next highest dose level. If data are not yet available for one or more of the first three participants and no DLT has been observed, or if one DLT has been observed, the new participant is entered at the same dose level. Lastly, if two or more DLTs have been observed, the dose level is de-escalated. This process is repeated for participants 5 and 6.

A patient who is inevaluable for toxicity is replaced with the next available patient if de-escalation rules have not been fulfilled when the next available patient is enrolled onto the study.

For a Compound A dose to be evaluated in Phase 1b, a total of six patients must have been treated at that dose with ≤1 evaluable patient experiencing at DLT. Based on safety, tolerability, pharmacokinetic (PK), pharmacodynamic, and efficacy data, intermediate doses may be explored as well as doses below 113 mg TID. The dose is confirmed after review of the safety, tolerability and PK data for patients who are both DLT- and PK-evaluable.

### Signal Seeking (Phase 1b)

When a dose is confirmed, the Phase 1b signal seeking portion of the study begins. Twenty evaluable patients are enrolled at the confirmed dose level (6 from Phase 1a and 14 from Phase 1b) to investigate the Compound A anti-tumor activity in patients with CRC by assessing the disease control rate (DCR) and overall response rate (ORR). The DCR is calculated once all patients receive 6 cycles of the selected Compound A dose. (DCR₆).

There are both superiority and futility boundaries in Phase 1b. The superiority boundary is based on ORR, and the futility boundary is based on DCR₆. If the posterior probability of ORR>10% is greater than 70%, then the study will proceed to the Phase 2 portion. If the superiority boundary is not met (i.e., the posterior probability of ORR>10% is not greater than 70%), then the futility boundary is evaluated. If the posterior probability of DCR₆≤30% is greater than 80%, then the study will not proceed to the Phase 2 portion. If the posterior probability of DCR₆≤30% is not greater than 80%, then the study will proceed to the Phase 2 portion.

### Phase 2

One hundred and two patients are enrolled into the Phase 2 Signal Confirmation portion of the study and are randomized 2:1 to Compound A or either LONSURF® or STIVARGA®. Patient responses to therapy are evaluated by blinded radiographic review and patients are followed for progression-free survival (PFS).

### Study Objectives and Endpoints

### Phase 1

The primary objectives of Phase I are as follows: 1) to determine the safety, tolerability, MTD, and RP2D of Compound A in patients with CRC; and 2) to assess the anti-tumor effects of Compound A by investigator assessment. The secondary objective of Phase I is to characterize the PK parameters of Compound A. The exploratory objective of Phase I is to assess the anti-tumor effects of Compound A.

The primary endpoints of Phase 1 include: 1) the occurrence of dose limiting toxicities (DLTs), 2) the frequency, duration, and severity of treatment-emergent adverse events (TEAEs), treatment-related TEAEs (TRAEs) and serious adverse events (SAEs), 3) incidence and shifts of clinically significant clinical laboratory abnormalities, 4) change from baseline in other observations related to safety, including electrocardiogram (ECG) and vital signs, 5) DCR₆, and 6) ORR. The secondary endpoints include the following PK parameters: maximum plasma concentration (Cₘₐₓ), time to maximum plasma concentration (Tₘₐₓ), area under the plasma concentration versus time curve (AUC) from time 0 to t (AUC₀₋ₜ), AUC from time 0 to 24 hours (AUC₀₋₂₄), apparent oral clearance (CL/F), apparent volume of distribution (Vz/F), and half-life (t_{1/2}). The exploratory endpoint is to evaluate changes in reduction in circulating tumor DNA via Guardant360 assay.

### Phase 2

The primary objective of Phase 2 is to assess the anti-tumor effects of Compound A by blinded radiographic review. The secondary objectives include: 1) evaluate short- and long-term safety, 2) characterize the PK parameters of Compound A, 3) assess overall survival, 4) assess the anti-tumor effects of Compound A by blinded radiographic review, and 5) assess the anti-tumor effects of Compound A by investigator assessment. The exploratory objective is to assess the anti-tumor effects of Compound A.

The primary endpoint of Phase 2 is: progression-free survival (PFS). The secondary endpoints of Phase II include: 1) frequency, duration, and severity of TEAEs, TRAEs, and SAEs, 2) incidence and shifts of clinically significant clinical laboratory abnormalities, 3) change from baseline in other observations related to safety, including ECGs and vital signs, 4) PK parameters, including: maximum plasma concentration (Cₘₐₓ), time to maximum plasma concentration (Tₘₐₓ), area under the plasma concentration versus time curve (AUC) from time 0 to t (AUC₀₋ₜ), AUC from time 0 to 24 hours (AUC₀₋₂₄), apparent oral clearance (CL/F), apparent volume of distribution (Vz/F), and half-life (t_{1/2}), 5) overall survival (OS), 6) DCR₆, 7) ORR by Response Evaluation Criteria in Solid Tumors (RECIST), version (v) 1.1, and 8) duration of response (DOR). The exploratory endpoint for Phase 2 is to evaluate changes in reduction in circulating tumor DNA via Guardant360 assay.

Compound a Dose and Administration of Compound a with Food

### Compound A Dose

The dosing strategy is intended to maximize AUC and Cₘᵢₙ while constraining Cₘₐₓ. The starting dosage of Compound A is 163 mg TID, with escalation to 226 mg TID or, if needed, de-escalation to 113 mg TID.

The rationale for these doses is based on PK data from 53 patients treated in Arm A (no strong cytochrome P450 CYP3A4 inhibitor) and Arm B (with a strong CYP3A4 inhibitor) of Study Compound A which were analyzed with a preliminary population PK model. The model was a one-compartment PK model with first-order absorption (ka) and lag time (Tlag) that described the individual PK data. The data confirmed the expected effect of a strong CYP3A4 inhibitor comedication in Arm B on the drug elimination rate (k) and apparent volume of distribution (Vz/F).

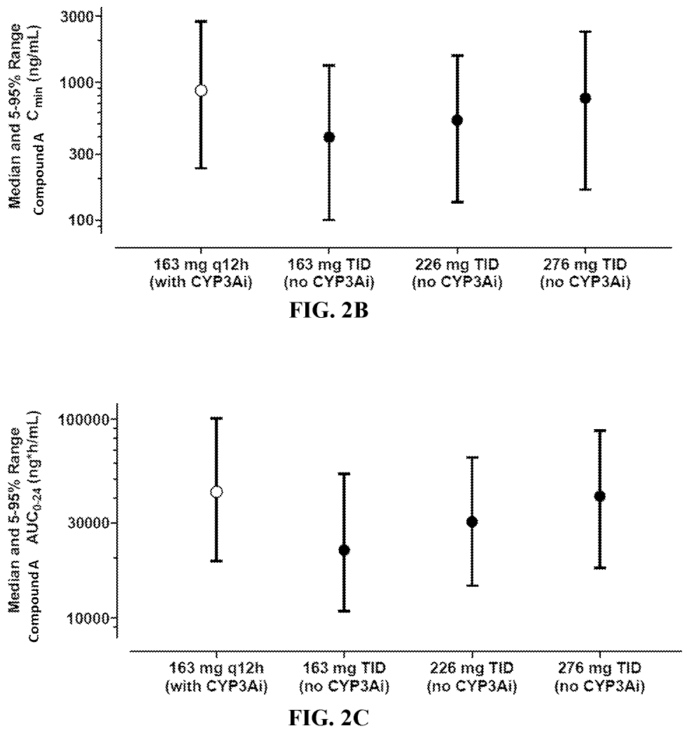

This model was used to compare the predicted PK of several dosages of Compound A TID without a CYP3A4 inhibitor to the predicted PK for administration of 163 mg Compound A every 12 hours (q12 h) with a strong CYP3A inhibitor, which is the recommended Phase 2 dose (RP2D) of Study Compound A. These data are shown in FIGS. 2A-C and Table 3A. The starting dose of 163 mg TID of Compound A alone was selected because it provides Cₘₐₓ, Cₘᵢₙ, as shown in FIGS. 2A-B, and AUC₀₋₂₄ approximately half that of 163 mg q12 h with a strong CYP3A4 inhibitor, as shown in FIG. 2C; the lower exposure provides a margin of safety in case CRC patients are more sensitive to the effects of Compound A than acute myeloid leukemia patients. The dose of 276 mg TID is expected to show exposure similar to that for 163 mg q12 h with a strong CYP3A inhibitor based on 3 key PK parameters (Cₘₐₓ, Cₘᵢₙ, and AUC₀₋₂₄), as shown in FIGS. 2A-C and Table 3A.

TABLE 3A
The quantitative values for each of the various pharmacokinetic
parameters at each dose of Compound A.

| | | Dosing | Simulated Median and 5-95% Range | | |
|---|---|---|---|---|---|
| | Dose | Fre- | Cₘₐₓ | Cₘᵢₙ | AUC₀₋₂₄ |
| Arm | (mg) | quency | (ng/mL) | (ng/mL) | (ng*h/mL) |
| A | 163 | TID | 1725 | 400 | 21,987 |
| | | | (1006, 3341) | (100, 1323) | (10,837, 53,056) |
| | 226 | | 2445 | 531 | 30,466 |
| | | | (1369, 4317) | (134, 1567) | (14,542, 64,223) |
| | 276 | | 3062 | 764 | 40,943 |
| | | | (1707, 5382) | (166, 2329) | (17,920, 87,518) |
| B | 163 | Q12h | 3228 | 871 | 43,128 (19,345, |
| | | | (1735, 6141) | (237, 2758) | 100,920) |

Abbreviations: Cₘₐₓ = maximum observed concentration; Cₘᵢₙ = minimum observed concentration; AUC₀₋₂₄ = area under the plasma concentration versus time curve from time 0 to 24 hours; q12h = every 12 hours; TID = 3 times a day.

Compound A Administration with Food

When Compound A is given TID, administering it consistently under fasted conditions is not practical; and given the available data, it appears unnecessary to administer Compound A under fasted conditions. Furthermore, administering Compound A with food in this study provides information regarding the effect of food.

The doses for LONSURF® and STIVARGA® are being used according to their respective labels which are incorporated herein by reference.

TABLE 3B
Inhibitors of CYP3A4

| Strong inhibitors¹ | Moderate inhibitors² |
|---|---|
| Boceprevir, Clarithromycin, Conivaptan | Amprenavir, Aprepitant, |
| Grapefruit juice, Indinavir, Itraconazole, | Atazanavir, Ciprofloxacin, |
| Ketoconazole, Lopinavir, Mibefradil, | Darunavir, Diltiazem, |
| Nefazodone, Nelfinavir, Posaconazole, | Erythromycin, Fluconazole, |
| Ritonavir, Saquinavir, Telaprevir, | Fosamprenavir, |
| Telithromycin, Voriconazole | Imatinib, Verapamil |

¹Increases the AUC of the substrate by ≥5-fold.
²Increases the AUC of the substrate by 2- to 5-fold.

TABLE 4
Inducers of CYP3A4

| Strong inducers¹ | Moderate inducers² |
|---|---|
| Avasimibe, Carbamazepine, | Bosentan, Efavirenz, |
| Enzalutamide, | Etravirine, Modafinil, |
| Phenytoin, Rifampin, St. John's wort | Nafcillin |

¹Decreases the AUC of the substrate by ≥80%.
²Decreases the AUC of the substrate by 50%-80%

### Study Periods and End of Study Definition

The “Screening period” is up to 21 days prior to the first dose of study intervention. During the “Treatment period” patients may continue to receive Compound A until disease progression or until they experience unacceptable toxicity. The “Safety follow-up period” is up to 30 days after the last administration of study intervention and as required, any further follow-up of ongoing serious adverse events (SAEs) and/or nonserious adverse events (AEs) considered to be reasonably or possibly related to study drug until either resolution or determined by the Investigator to be chronic or stable. The “Survival follow-up” is from the end of the safety follow-up period for a maximum of 5 years. The “End of Study” is the final contact with the last patient for survival follow-up.

### Number of Patients

Thirty-two patients are enrolled, with 6 patients in up to 3 dose level cohorts in Phase 1a and an additional 14 patients in the Phase 1b Signal seeking portion at a dose deemed safe and tolerable in Phase 1a. With a flat prior Beta (0.5, 0.5), the sample size of 20 evaluable patients for signal seeking (6 from Phase 1a and 14 from Phase 1b) will provide the following operating characteristics based on the superiority and futility boundaries specified as follows: the probability of proceeding to the Phase 2 portion due to superiority on ORR is 79% and 8% if the true ORR is 20% and 5%, respectively, while the probability of not proceeding to the Phase 2 portion due to futility on DCR₆ is 63% and 5% if the true DCR₆ is 20% and 40%, respectively.

Patients who have discontinued treatment prior to their first post-baseline disease assessment for reasons other than progression, TEAE, or death are replaced.

For Phase 2, 78 PFS events are needed to provide 80% power with a 1-sided alpha of 0.1, assuming Compound A improves the treatment effect in PFS from 3 months to 5 months (hazard ratio=0.6) with 2:1 randomization ratio. One hundred two total patients are enrolled in 12 months and followed for 4 months for the PFS analysis.

### Study Interventions Administered

Patients enrolled in the Phase 1 portion of the study and to the Compound A arm of the Phase 2 portion of the study receive Compound A. Compound A is a small molecule formulated into capsule (of strength 25 mg or 113 mg) or oral solution. Patients in Phase 1 and the Compound A arm of Phase 2 receive Compound A from Cycle 1 Day 1, continuously in 28-day cycles, throughout study at a dose and schedule as determined by assigned dose level. Based on emerging data, intermediate doses may be explored as well as doses below 113 mg TID. Compound A administration continues until receipt of prohibited concomitant medication, development of progressive disease, or unacceptable toxicity. Compound A is administered with food. Patients taking capsules take the calculated number of capsules with water. Patients taking oral solution take the calculated volume of solution followed by a water rinse, both drawn into oral syringe. Doses should be taken no less than 6 hours after the previous dose.

### Compound A Dose

The starting dose of Compound A is 163 mg TID (489 mg total daily dose). The Compound A starting dose is escalated or de-escalated according to the dose levels provided in Table 5.

TABLE 5
CRC Dosing Schedule using Compound A

| Dose Level | Dose |
|---|---|
| −3 | 25 mg TID |
| −2 | 50 mg TID |
| −1 | 113 mg TID |
| 1ª | 163 mg TID |
| 2 | 226 mg TID |
| 3 | 276 mg TID |

^{a}starting dose; TID = 3 times a day.

### Chemotherapy

Patients enrolled into the chemotherapy arm in the Phase 2 portion of the study receive one of 2 different chemotherapy agents either LONSURF® or STIVARGA®. The dose, supportive care/hydration and modifications to dose are according to the product labels.

### Compound a Dosing Based on Serum Electrolytes

Potassium should be maintained at ≥4.0 mEq/L and magnesium at ≥2.0 mEq/L. Maintenance oral supplementation should be started for patients whose potassium and magnesium levels are below these thresholds, and Compound A dosing instructions as provided should be followed.

### DLT Definition

A DLT is defined as any of the following events that is at least possibly related to study drug as assessed by the investigator. The DLT observation period for the purposes of dose-escalation is the first cycle (28 days) of therapy.

A “hematologic toxicity” event includes any of the following: 1) Grade 4 neutropenia (ANC<<500 cells/mm³) unresolved to Grade 2 (ANC>1500 cells/mm³) or baseline for more than 7 consecutive days in the absence of growth factor support, 2) ≥Grade 3 neutropenia (ANC<1000 cells/mm³) with a single temperature of >38.3° C. (101° F.) or a sustained temperature of ≥38° C. (100.4° F.) for more than 1 hour, 3) Grade 4 thrombocytopenia of any duration, 4) ≥Grade 3 thrombocytopenia (<50,000/mm³) with clinically significant bleeding, or 5) Grade 4 anemia.

A “nonhematologic toxicity” event includes any of the following: 1) any clinically significant ≥Grade 3 nonhematologic toxicity, as defined in the CTCAE, version 5.0, or 2) any Grade 3 QTcF prolongation (≥501 ms average of the triplicate ECGs).

A “general” DLT includes any of the following: 1) any hematologic or nonhematologic toxicity, regardless of the NCI CTCAE grade, resulting in Compound A discontinuation or interruption of Compound A for >7 consecutive days or receipt of <75% of planned dose intensity, or 2) any patient who requires more than a 2-week delay prior to beginning Cycle 2 due to a TRAE is considered to have experienced a DLT.

The following events are not considered DLTs: 1) Grade 2 allergic reaction necessitating discontinuation of study drug will not be considered a DLT and patient is replaced, or 2) rapidly progressive disease requiring discontinuation of Compound A during the DLT period is not considering a DLT and patient is replaced.

Concomitant Therapy with Compound A

All prior and concomitant mediations are captured from 56 days before Cycle 1 Day 1 (C1D1) up until the Safety Follow-up visit.

Prohibited Concomitant Therapies and Medications with Compound A

Radiation therapy is prohibited during study participation and the following medications and treatments are prohibited during study participation: 1) any commercially available or investigational CRC therapy other than Compound A, 2) systemic immunosuppressive therapy except for glucocorticoids for physiologic replacement (equivalent to ≤10 mg per day of prednisone for patients ≥18 years or ≤10 mg/m²/day for patients <18 years). Short-term use of glucocorticoids is permitted for prevention or treatment of allergic and transfusion reactions or complications due to other evolving medical conditions, 3) moderate or strong CYP3A4 inhibitors or inducers, as shown in Tables 3B and 4, or 4) medications known or suspected to prolong the QT interval/QTc except for drugs with low risk of QT interval/QTc prolongation that are used as standard supportive therapies (e.g., diphenhydramine, famotidine, ondansetron).

Any medications and treatments other than those listed above, including palliative and supportive care for disease-related symptoms, are permitted during the study. Patients are closely monitored, and treatment instituted for disease-related symptoms, as appropriate. Antiemetic treatments are used at discretion and in accordance with the American Society of Clinical Oncology guidelines or equivalent after documented nausea or vomiting has occurred without medications having been used.

Concomitant Therapy with LONSURF or STIVARGA

Patients receiving LONSURF or STIVARGA should follow the respective USPI for prohibited and permitted concomitant medications.

### Dose Modification

For patients requiring dose reductions, step decreases occur in single dose level reductions, and up to 2 dose reductions are allowed. No dose reductions are allowed below Dose Level-3, and if required, Compound A is to be discontinued. Compound A interruptions longer than 28 days require discussion before resuming therapy. In Cycle 1 doses may be held, but may not be reduced, unless the patient has experienced a DLT. A patient may only begin a new cycle if their ANC is ≥1,000 and their platelets are ≥50,000. Any nonhematologic toxicity that are definitely due to Compound A are graded and managed as outlined in Table 6A. The rules for management of dose modifications for hematologic toxicity are outlined in Table 6B. No dose escalation above the patient's initial dose assignment is permitted, and no intervention after the completion of the study participation is planned.

Tables 6A and 6B depict the dose modification of Compound A for the management of toxicities.

TABLE 6A
Action for nonhematologic toxicity criteria.

| Criteria | Action |
|---|---|
| ≥Grade 3 drug-related | Permanently discontinue Compound A |
| allergic reaction | |
| Grade 3 arrhythmia | Permanently discontinue Compound A |
| Other ≥Grade 3 | Withhold treatment and resume |
| treatment-related | Compound A upon recovery |
| nonhematologie | to Grade 1 or baseline, under |
| toxicity | the following directions: |
| | If recovered in ≤7 days of |
| | treatment interruption, restart |
| | Compound A at the original dose level. |
| | If recovered in >7 to 14 days |
| | of treatment interruption, |
| | restart Compound A at the |
| | next lower dose level. |
| | If recovered in >14 days of |
| | treatment interruption, |
| | treatment may resume at the |
| | next lower dose level only |
| | after discussion with the |
| | Study Responsible Physician if, in the |
| | opinion of the Investigator, the patient is |
| | benefitting from treatment. |
| ≥Grade 2 QTcP | Withhold treatment. Compound A |
| prolongation | may be resumed as soon as |
| ≥481 ms | the QTcF has returned to Grade 1: |
| | Patients who experience a |
| | Grade 3 QTcF prolongation |
| | should restart Compound A |
| | at the next lower dose level. |
| | Patients who experience a |
| | Grade 2 QTcF prolongation |
| | should restart Compound A |
| | without dose modification. |

TABLE 6B
Action for hematologic toxicity criteria.

| Criteria | Action |
|---|---|
| Neutropenia (ANC) | |
| Grade 3 | |
| (ANC 500-999 | Wi [symbol] hold dose until resolved to ≤ Grade 2 |
| cells/mm³) | (ANC ≥1500 cells/mm³), then: |
| | If resolved in ≤7 days, then maintain dose level |
| | If resolved in >7 days, then dose |
| | reduce by 1 dose level |
| | If recurred, then dose reduce by 1 dose level |
| Grade 4 | Withhold dose until resolved to |
| (ANC <500 cells/mm³) | ≤ Grade 2 (ANC ≥1500 cells/mm³), |
| Febrile neutropenia | then dose reduced by 1 dose level |
| (ANC <1000 cells/mm³, | Dose withheld until resolved to |
| with a single temperature | ≤ Grade 2 (ANC ≥1500 cells/mm³) |
| of >38.3° C. | and fever/infection recovered, |
| (101° F.) or a sustained | then reduce by 1 dose level |
| temperature of | |
| ≥38° C. (100.4° F.) for | |
| more than 1 hour) | |
| Thrombocytopenia (PLT) | Withhold dose until resolved to ≤ Grade 2 |
| Grade 3 (PLT | (PLT ≥75,000 cells/mm³), then: |
| 25,000-49,999 cells/mm³) | If resolved in ≤7 days, then |
| Grade 4 (PLT | maintain dose level |
| <25,000 cells/mm³) | If resolved in >7 days, then |
| | dose reduce by 1 dose level |
| | Withhold dose until resolved to |
| | ≤ Grade 2, then reduce by 1 dose |
| | level |
| Anemia | Withhold dose until resolved to ≤ Grade 2, then: |
| Grade 3 | If resolved in ≤7 days, then maintain dose level |
| Grade 4 | If resolved in >7 days, then dose |
| | reduce by 1 dose level |
| | Withhold dose until resolved to |
| | ≤ Grade 2, then reduce by 1 dose |
| | level if patient is not discontinued |

Abbreviations: ANC = absolute neutrophil count, PLT = platelets.

### Study Population

### Inclusion Criteria

Patients with R/R CRC are enrolled in this study. Patients meeting all of the following criteria are eligible for the study:

### Diagnosis

Patients with microsatellite stable/proficient mismatch repair (MSS/pMMR) mCRC (immunohistochemistry, polymerase chain reaction or next-generation sequencing can be used), which is radiographically measurable by RECIST v1.1.

### Disease Status

Evidence of locally recurrent or metastatic disease based on imaging studies (e.g, computed tomography [CT], magnetic resonance imaging [MRI]) within 28 days before the first study drug dose. At least 1 measurable lesion ≥20 mm by conventional techniques or ≥10 mm by spiral CT scan or MRI, with the last imaging performed within 28 days before the first study drug dose. If there is only 1 measurable lesion and it is located in previously irradiated field, it must have demonstrated unequivocal progression. Patients must have had at least one line of standard-of-care therapy and must have progressed on or been intolerant to or unable to receive oxaliplatin, irinotecan, and bevacizumab in the advanced/metastatic setting. If the patient has a left-sided primary tumor that was RAS wild-type, the patient must have received anti-EGFR therapy. Patients must have experienced documented unequivocal progressive disease by either RECIST v1.1 or clinical assessment.

### Age

Male and female patients must be ≥18 years of age.

### Performance Level

Eastern Cooperative Oncology Group (ECOG) performance status score of 0 to 1.

### Prior Therapy

If a patient is receiving radiation therapy, the patient has had a 2-week washout period following completion of the treatment prior to receiving the first study drug dose and the patient continues to have at least 1 measurable lesion, per above criterion. For patients that have received immunotherapy, at least 42 days have past since prior immunotherapy, including tumor vaccines and checkpoint inhibitors, and at least 21 days have past prior to receipt of chimeric antigen receptor therapy or other modified T-cell therapy. At least 7 days or 5 half-lives, whichever is longer, must have past since the completion of therapy with a biologic agent.

### Adequate Organ Function Requirements

A patient must have a bone marrow reserve defined by: absolute neutrophil count (ANC)≥1,500/μL, platelet count ≥100,000/μL, and hemoglobin ≥9 g/dL. A patient must have hepatic function defined by: total bilirubin≤1.5× the upper limit of normal (ULN); alanine aminotransferase (ALT) or aspartate aminotransferase (AST)≤2.5×ULN (≤5×ULN if the elevation can be reasonably ascribed to the presence of metastatic disease in liver). A patient must have renal function defined by: estimated glomerular filtration rate ≥50 mL/min based on the Cockcroft-Gault formula for adults. A patient must meet a coagulation functional threshold defined by an international normalized ratio (INR) or prothrombin time (PT)≤1.5×ULN, unless the patient is receiving anticoagulant therapy, as long as PT or partial thromboplastin time (PTT) is within therapeutic range of intended use of anticoagulants. A patient must have an activated PTT (aPTT)≤1.5×ULN, unless the patient is receiving anticoagulant therapy, as long as PT or PTT is within therapeutic range of intended use of anticoagulants. A patient must have potassium maintained at ≥4.0 mEq/L and magnesium at ≥2.0 mg/dL.

### Contraception

Females of childbearing potential must use a highly effective method of contraception or double barrier method from the time of study enrollment to an endpoint defined by the treatment group enrolled. The endpoint for each treatment is defined as: 120 days following the last study drug dose of Compound A, 2 months following the last study drug dose of Stivarga, and up to at least 6 months following the last study drug dose of Lonsurf. Fertile males agree to use barrier contraception from the time of enrollment through 120 days after the last study drug dose.

### Exclusion Criteria

Patients with a known history of HIV 1/2 antibodies must have viral load testing prior to study enrollment. Patients meeting any of the following criteria were not eligible for the study participation: 1) having a known microsatellite instability high (MSI-H) CRC; 2) having a prior history of malignant bowel obstruction requiring hospitalization in the 6 months prior to enrollment; 3) having a history of uncontrolled ascites, defined as symptomatic ascites and/or repeated paracenteses for symptom control in the past 3 months; 4) having detectable HIV viral load within the previous 6 months; 5) patient is Hepatitis B (defined as hepatitis B virus [HBV] surface antigen positive and HBV core antibody positive, with positive HBV DNA, or HBV positive core antibody alone with positive HBV DNA); or 6) patient is Hepatitis C (defined as positive hepatitis C [HCV] antibody with reflex to positive HCV RNA).

A patient cannot have any of the following within the 6 months prior to study entry: myocardial infarction, uncontrolled/unstable angina, congestive heart failure (New York Heart Association Classification Class ≥II), life-threatening, uncontrolled arrhythmia, cerebrovascular accident, or transient ischemic attack.

A patient has been excluded if they have any GI issue of the upper GI tract likely to affect oral drug absorption or ingestion (eg, gastric bypass, gastroparesis), or Cirrhosis with a Child-Pugh score of B or C.

A patient has been excluded if they have a brain metastasis, except for those patients who have completed definitive therapy, are not on steroids, have a stable neurologic status for at least 4 weeks after completion of the definitive therapy and steroids, and do not have neurologic dysfunction that would confound the evaluation of neurologic and other AEs. A patient has been excluded if they have a history of or any concurrent condition, therapy, laboratory abnormality, or allergy to excipients (see formulation details in Investigator Brochure) that might confound the results of the study, interfere with the patient's ability to participate for the full duration of the study, or not be in the best interest of the patient to participate.

Patients that have received prior chemotherapy, targeted small molecule therapy, or radiation therapy within 2 weeks prior to study baseline or who has not recovered (ie, ≤Grade 1 or at baseline) from AEs related to a previously administered agent have been excluded from the study.

Patients with ≤Grade 2 neuropathy or ≤Grade 2 alopecia are an exception to this criterion and may qualify for the study. If patient underwent major surgery, they must have recovered adequately from the toxicity and/or complications from the intervention prior to starting therapy.

Patients that have received transfusion of blood products (including platelets or red blood cells) or administration of colony stimulating factors (including granulocyte-colony stimulating factor [G-CSF], granulocyte macrophage-colony stimulating factor [GM-CSF], or recombinant erythropoietin) within 4 weeks of the first dose of study drug have been excluded.

Patients with any other known additional malignancy that is progressing or requires active treatment (excluding adequately treated basal cell carcinoma, squamous cell of the skin, cervical intraepithelial neoplasia [CIN]/cervical carcinoma in situ or melanoma in situ or ductal carcinoma in situ of the breast) have been excluded. A prior history of other cancer is allowed, as long as there is no active disease within the prior 5 years.

The concurrent use of moderate and strong inhibitors or inducers of CYP3A4 should be discontinued at least 7 days prior to enrollment, to prevent patient exclusion.

Patients that have participated in another therapeutic interventional clinical study in which an investigational agent was administered within 30 days before starting Compound A have been excluded. Patients may continue with noninterventional follow-up from previous clinical studies.

### Statistical Considerations

There is no formal statistical hypothesis testing in Phase 1. The Phase 2 portion will test the following primary and secondary hypotheses: 1) H₀: hazard ratio for PFS (Compound A vs. control) is greater than or equal to 1 vs Hₐ: hazard ratio for PFS is less than 1; and 2) H₀: hazard ratio for OS (Compound A vs. control) is greater than or equal to 1 vs Hₐ: hazard ratio for OS is less than 1.

### Sample Size Determination

The actual number of patients enrolled in Phase 1 depends on the number of dose level(s) at which toxicities are seen and the number of dose levels evaluated in dose escalation. Up to 32 patients are enrolled in Phase 1, with 6 patients in up to 3 dose level cohorts in Phase 1a and an additional 14 patients in the Phase 1b Signal seeking portion at a dose deemed safe and tolerable in Phase 1a.

With a flat prior Beta (0.5, 0.5), the sample size of 20 evaluable patients for signal seeking (6 from Phase 1a and 14 from Phase 1b) will provide the following operating characteristics based on the superiority and futility boundaries.
The probability of proceeding to the Phase 2 portion due to superiority on ORR is 79% and 8% if the true ORR is 20% and 5%, respectively.
The probability of not proceeding to the Phase 2 portion due to futility on DCR6 is 63% and 5% if the true DCR6 is 20% and 40%, respectively. Patients who have discontinued treatment prior to their first post-baseline disease assessment for reasons other than progression, TEAE, or death are replaced.

For Phase 2, 78 PFS events are needed to provide 80% power with a 1-sided alpha of 0.1, assuming Compound A will improve the treatment effect in PFS from 3 months to 5 months (hazard ratio=0.6) with 2:1 randomization ratio. One hundred two total patients are enrolled in 12 months and followed for 4 months for the PFS analysis.

### Efficacy Analyses

For Phase 1, efficacy analyses are conducted in the evaluable population and are descriptive and graphical in nature. No formal statistical hypothesis testing is performed. The point estimates and 95% exact binomial confidence intervals are provided for ORR and DCR6. DOR is summarized using Kaplan-Meier method.

For Phase 2, efficacy analyses are based on the ITT population. PFS and OS are tested in a sequential fashion. If the treatment effect in PFS is statistically significant, the treatment effect in OS is tested. Log-rank test is used to test PFS and OS effects. Chi-square test is used to test ORR and DCR6. The populations for analyses are summarized in Table 6C.

TABLE 6C
Populations for Analyses

| Population | Description |
|---|---|
| Intention-to-treat | For Phase 1: All patients enrolled |
| (ITT) | who provide informed consent. |
| | For Phase 2: All randomized patients. |
| DLT evaluable | All patients with data used for |
| | implementing the dose-escalation |
| | schedule (Phase la). These patients |
| | will have received 75% of all |
| | study drug doses in first cycle (C1) |
| | (i.e., the DLT evaluation period) |
| | or stopped treatment because of |
| | DLTs in the DLT evaluation period. |
| Evaluable | All treated patients except for those |
| | who have discontinued treatment |
| | prior to their first post-baseline |
| | disease assessment for reasons other |
| | than progression, TEAE, or death. |
| Safety | Consists of all enrolled patients |
| | who received at least 1 dose of |
| | Compound A during the study |
| PK | All patients who receive at least one |
| | dose of Compound A and have at |
| | least one valid plasma concentration |
| | of Compound A determined. |
| Pharmacodynamic | Patients who are exposed to Compound |
| | A and have sufficient samples |
| | collected to permit pharmacodynamic analyses. |

### Statistical Analyses

The safety and efficacy analyses are presented by study phase. If appropriate, summary tabulations are presented that display the number of observations, mean, standard deviation, median, minimum, and maximum for continuous variables, and the number and percentage per category for categorical data. Time-to-event endpoints are analyzed using Kaplan-Meier method.

### Disposition

The number of patients included in each analysis set is summarized, along with the reason for any exclusions. Reasons for discontinuing from study treatment and/or withdrawing from study participation are summarized.

### Demographics and Baseline Characteristics

Descriptive summaries of demographic and baseline characteristics are tabulated.

### Exposure

The overall duration of study treatment administration and number of cycles initiated is summarized. For each patient, the relative dose intensity of study drug is calculated. These data are further summarized for all patients at the dose selected for Phase 1b by calculating the mean, standard deviation, median, and range of these values. The number and proportion of patients with 1 or more dose modifications (i.e., dose reduction) are tabulated along with the reason for modification.

### Efficacy Analyses

For Phase 1, efficacy analyses are conducted in the evaluable population and are descriptive and graphical in nature. The point estimates and 95% exact binomial confidence intervals are provided for ORR and DCR6. DOR is summarized using Kaplan-Meier method. For Phase 2, efficacy analyses are based on the ITT population. PFS and OS are tested in a sequential fashion. If the treatment effect in PFS is statistically significant, the treatment effect in OS is tested. Log-rank test is used to test PFS and OS effects. Chi-square test is used to test ORR and DCR6.

### Safety Analyses

All safety analyses are performed on the Safety Population. The safety assessment is based on the frequency of AEs, on the observation of clinically significant abnormalities of laboratory values, concomitant medication use, vital signs, ECGs, ECOG performance status, and physical examination data in the Safety Population.

AEs are coded using the standard Medical Dictionary for Regulatory Activities, version 19.0 or higher, and graded. Analyses of AEs is based on the principle of treatment emergence. Treatment-emergent AEs are defined as having onset after study drug dosing or a sign, symptom, or diagnosis that worsens after study drug dosing. Whenever an analysis or summary of AEs is mentioned, it is intended that this is in reference to TEAEs, unless it is stated otherwise.

TEAEs are summarized based on the number and percentage of patients experiencing the event by system organ class and preferred term. The causal relationship between the occurrence of an AE and study drug is determined (ie, Related, Probably Related, Possibly Related, Unlikely Related and Not Related). If a patient experiences repeat episodes of the same AE, then the event with the highest severity grade and strongest causal relationship to study drug is used for purposes of incidence tabulations.

Tabular summaries or listings is provided for: 1) all TEAEs, 2) TEAEs by relationship to study drug and maximum severity grade, 3) TEAEs with action of study drug interrupted or dose reduced, 4) TEAEs with action of study drug discontinued, 5) Adverse events of special interest (AESIs), and 6) SAEs.

Hematology and serum chemistries are summarized in a descriptive manner by calculating the mean, standard deviation, median, and range. Directional shifts in laboratory toxicity grades (comparing baseline grade with worst postbaseline grade) are analyzed using standard shift tables, presenting number and proportion of patients and their maximum grade shift. For analytes without a toxicity grading scale, the shift table will present directional shifts from baseline to above or below the laboratory standard normal range using the maximum increase and/or decrease observed throughout the study. Vital signs are summarized in a descriptive manner by calculating the mean, standard deviation, median, and range in the same manner described for laboratory values. Frequency statistics are presented for ECOG performance status.

Descriptive statistics for the actual values and changes from baseline in ECGs are tabulated by time point.

QTc is calculated using Fridericia's correction (QTcF). The formulas are: QTcF=QT/(RR0.33), where RR=60/heart rate (beats per minute). A categorical analysis of maximum QTcF and maximum changes from baseline is performed by summarizing the number and percentage of patients in each QTcF category (<450 ms, 450-480 ms, 481-500 ms, and ≥500 ms) and in categories of changes from baseline (≥30 ms and ≥60 ms).

### Pharmacokinetic Analyses

Plasma concentrations of Compound A are determined with a validated bioanalytical assay. The following PK parameters (where applicable) are calculated using standard noncompartmental methods.

Whenever possible, the following parameters are calculated for each patient, based on the plasma concentrations of Compound A (and any relevant metabolites): 1) AUC_{0-inf} ((area under the plasma concentration versus time curve from time 0 extrapolated to infinity, calculated as follows: AUC_{0-inf}=AUC₀₋ₜ+(Cₗₐₛₜ÷λz), where Cₗₐₛₜ is the last quantifiable concentration, and λz is the apparent terminal elimination rate constant)), 2) AUC₀₋ₜ: (area under the plasma concentration versus time curve from time 0 to the last measurable concentration, calculated using the linear trapezoidal rule for increasing and decreasing concentrations), 3) AUC₀₋₂₄ (area under the plasma concentration versus time curve from time 0 to 24 hours, estimated as three times AUC₀₋ₜ with TID dosing), 4) Cₘₐₓ (maximum observed concentration), 5) Tₘₐₓ (time to maximum observed concentration), 6) t_{1/2} ((apparent terminal elimination half-life (whenever possible), where t_{1/2}=ln(2)/λz), 7) CL/F (apparent oral clearance), and 8) Vz/F: apparent volume of distribution during the terminal phase. Summary statistics for plasma concentrations and PK parameters are generated as appropriate. PK parameters are summarized by dose.

### Adverse Events

An AE is any untoward medical occurrence in a patient or clinical study participant, temporally associated with the use of study drug, whether or not considered related to the study drug. Events that meet the AE definition may include: 1) any abnormal laboratory test results (hematology, clinical chemistry, or urinalysis) or other safety assessments (e.g., ECG, radiological scans, vital signs measurements), including those that worsen from baseline, considered clinically significant in the medical and scientific judgment (i.e., but not related to progression of underlying disease), 2) exacerbation of a chronic or intermittent preexisting condition including either an increase in frequency and/or intensity of the condition, 3) new condition detected or diagnosed after study drug administration even though it may have been present before the start of the study, 4) signs, symptoms, or the clinical sequelae of a suspected drug-drug interaction, 5) signs, symptoms, or the clinical sequelae of a suspected overdose of either study drug or a concomitant medication. Overdose per se will not be reported as an AE/SAE unless it is an intentional overdose taken with possible suicidal/self-harming intent. Such overdoses should be reported regardless of sequelae, or 6) “lack of efficacy” or “failure of expected pharmacological action” will not be reported as an AE or SAE. Such instances are captured in the efficacy assessments. However, the signs, symptoms, and/or clinical sequelae resulting from lack of efficacy are reported as AEs or SAEs if they fulfill the definition of an AE or SAE. Events that do not meet the AE definition may include: 1) any clinically significant abnormal laboratory findings or other abnormal safety assessments that are associated with the underlying disease, unless judged by the Investigator to be more severe than expected for the participant's condition, 2) the disease/disorder being studied or its progression, signs, or symptoms, unless more severe than expected for the participant's condition, 3) medical or surgical procedure (e.g., endoscopy, appendectomy): the condition that leads to the procedure is the AE, 4) situations in which an untoward medical occurrence did not occur (social and/or convenience admission to a hospital), or 5) anticipated day-to-day fluctuations of preexisting disease(s).

If an event is not an AE per the definition above, then it cannot be an SAE even if serious criteria are met (e.g., hospitalization for signs/symptoms of the disease under study, death due to progression of disease). An SAE is defined as any untoward medical occurrence that, at any dose: 1) results in death, 2) is life-threatening (the term “life-threatening” in the definition of serious refers to an event in which the participant was at risk of death at the time of the event. It does not refer to an event that hypothetically might have caused death if it were more severe), 3) requires inpatient hospitalization or prolongation of existing hospitalization (in general, hospitalization signifies that the participant has been detained (usually involving at least an overnight stay) at the hospital or emergency ward for observation and/or treatment that would not have been appropriate in the physician's office or outpatient setting. Complications that occur during hospitalization are AEs. If a complication prolongs hospitalization or fulfills any other serious criteria, the event is serious. When in doubt as to whether “hospitalization” occurred or was necessary, the AE should be considered serious. Hospitalization for PD, events caused by PD, and elective treatment of a preexisting condition that did not worsen from baseline are not considered AEs), 4) results in persistent disability/incapacity (the term disability means a substantial disruption of a person's ability to conduct normal life functions. This definition is not intended to include experiences of relatively minor medical significance, such as uncomplicated headache, nausea, vomiting, diarrhea, influenza, or accidental trauma (eg, sprained ankle), that may interfere with or prevent everyday life functions but do not constitute a substantial disruption), or 5) is a congenital anomaly/birth defect. Other situations may include: medical or scientific judgment should be exercised in deciding whether SAE reporting is appropriate in other situations such as important medical events that may not be immediately life-threatening or result in death or hospitalization but may jeopardize the participant or may require medical or surgical intervention to prevent one of the other outcomes listed in the above definition. These events should usually be considered serious. Examples of such events include invasive or malignant cancers, intensive treatment in an emergency room or at home for allergic bronchospasm, blood dyscrasias or convulsions that do not result in hospitalization, or development of drug dependency or drug abuse.

### Prolonged QTc

Prolongation of QTcF (average of triplicate ECGs) of CTCAE Grade 2 or higher is classified as an AESI. Prolonged QTc was observed in dogs treated with Compound A.

Common supportive care medications used in oncology patients also can cause QT interval prolongation, such as fluoroquinolones, azole antifungals, and 5-HT3 antagonists (refer to Section 6.6.1 for prohibited concomitant medications). Patients may be at risk of QT interval prolongation when such treatments are combined. It is recommended to obtain an ECG before and after beginning any new medication and after dose increases of concurrent medications known to prolong QTc and in patients presenting with vomiting and/or diarrhea.

Withhold treatment for any QTcF prolongation ≥481 ms. QTc is calculated using Fridericia's correction, with the reported QTcF being the average of the 3 distinct values from the patient's serial ECGs. Further management is guided by degree of prolongation. Address any other underlying causes of QT prolongation, including identifying and stopping other precipitating drugs and aggressive correction of electrolyte abnormalities. Causal relationship to Compound A should be evaluated in the context of concomitant medications and electrolyte imbalances.

### Assessment of Intensity

The intensity of TEAEs is determined according to the NCI CTCAE, version 5.0. TEAEs not listed in the NCI CTCAE, are assessed according to the intensity of each AE and SAE reported during the study and assigned to one of the following categories: 1) Grade 1 (mild): asymptomatic or mild symptoms; clinical or diagnostic observations only; intervention not indicated, 2) Grade 2 (moderate): minimal, local, or noninvasive intervention indicated; limiting age-appropriate instrumental activities of daily living, 3) Grade 3 (severe or medically significant but not immediately life-threatening): hospitalization or prolongation of hospitalization indicated; disabling; limiting self-care activities of daily living, 4) Grade 4 (life-threatening consequences): urgent intervention indicated, 5) Grade 5: death related to AE. An event is defined as serious when it meets at least one of the predefined outcomes as described in the definition of an SAE, NOT when it is rated as severe.

### Assessment of Causality

The relationship between the study drug and each occurrence of each AE/SAE is assessed. The relationship is determined using clinical judgement. A “reasonable possibility” of a relationship conveys that there are facts, evidence, and/or arguments to suggest a causal relationship, and not that a relationship cannot be ruled out. Alternative causes, such as underlying disease(s), concomitant therapy, and other risk factors, as well as the temporal relationship of the event to study drug administration, are considered and investigated. For each AE/SAE, the review of the AE/SAE and assessment of causality is documented. The causality assessment is one of the criteria used when determining regulatory reporting requirements.

### Genetics

Genetic variation may affect a participant's response to study drug, susceptibility to, and severity and progression of disease. Variable response to study drug may be due to genetic determinants that affect drug absorption, distribution, metabolism, and excretion; mechanism of action of the drug; disease etiology; and/or molecular subtype of the disease being treated. Tissue samples are collected for mutational status. Genetic research may consist of the analysis of one or more candidate genes or the analysis of genetic markers throughout the genome (as appropriate). Tumor tissue is analyzed for mutational status. Additional analyses may be conducted if it is hypothesized that this may help further understand the clinical data. The samples may be analyzed as part of a multistudy assessment of genetic factors involved in the response to Compound A or study drugs of this class to understand study disease or related conditions.

### Benefit-Risk Assessment

Off-target screening assays showed no cross-reactivity of Compound A against >125 molecular targets; therefore, treatment-related adverse events (TRAEs) are expected to be due to mechanism-based target effects. Potential main targeted organs of toxicity identified in the pivotal Good Laboratory Practice-compliant, 28-day rat and dog toxicology studies were the heart (electrocardiogram [ECG] changes in dogs), eyes (cataracts in rats only), liver, bone marrow, lymphoid tissues, male reproductive system, and female reproductive system. Safety measures are implemented to monitor and protect patients from these effects. Patients have regular ECG assessments throughout the study. Hepatic and endocrine function are routinely and frequently assessed. Conservative drug withholding and modification parameters are clearly outlined for events of QT prolongation or hepatic enzyme elevations.

### Study Assessments and Procedures

### Efficacy Assessments

For tumor measurements, all sites of disease, target and nontarget lesions are assessed at Baseline. Objective disease status is recorded at each evaluation. The same method of assessment and the same technique used for study screening (CT scan or MRI) to characterize each lesion is used at each subsequent post-screening assessment. Table 7 shows the disease response criteria for target and non-target lesions.

All patients in the study have measurable disease that has been radiologically documented within 28 days prior to initiating study drug treatment, defined as at least 1 measurable lesion with the following characteristics: 1) ≥10 mm in longest diameter on an axial image by CT scan or MRI with ≤5 mm reconstruction interval. If slice thickness is no greater than 5 mm, the longest diameter must be at least 2 times the thickness, 2) ≤20 mm longest diameter by chest X-ray (if clearly defined and surrounded by aerated lung); CT is preferred, even without contrast, or 3) lymph nodes ≥15 mm in short axis on CT scan (CT slice thickness of ≤5 mm). If there is only 1 measurable lesion and it is located in previously irradiated field, it must have demonstrated progression according to RECIST v1.1.

The following are considered non-measurable lesions and are captured: 1) masses <10 mm, 2) lymph nodes 10-14 mm in short axis, 3) Leptomeningeal disease, 4) ascites, pleural or pericardial effusion, 5) lymphangitic involvement of skin or lung, 6) abdominal mases or organomegaly identified by physical exam that cannot be measured by reproducible imaging techniques, 7) blastic bone lesions, and 8) both benign and equivocal (“cannot exclude”) findings should not be included.

### Target Vs Non-Target Lesions

Lesions are characterized as either “target” or “not target”. All measurable lesions up to a maximum of 2 lesions per organ and 5 lesions in total, representative of all involved organs, are identified as “target” lesions and measured and recorded at screening. Target lesions are selected on the basis of their size (i.e., those with the longest diameter) and suitability for accurate repeated measurement. The sum of the diameters for all target lesions is calculated and recorded as the baseline sum diameters. All other lesions not classified as target lesions (or sites of disease) are to be identified as “non-target” lesions.

### Scan Procedures

Contrast-enhanced CT scans of chest, abdomen, and as clinically indicated, pelvis are preferred for evaluation of disease status; however, a contrast-enhanced MRI can be performed if a patient has or develops allergy to iodinated contrast agents. Positron emission tomography (PET) alone is not acceptable as part of the imaging to be performed for this study. If PET is performed, the PET alone will not serve as a basis for patient removal from the study, as there can be false positives. PET progression must be confirmed with a CT/MRI. If a combined 18F-deoxyglucose (FDG) PET-CT scan is performed, the CT portion of that examination should not be substituted for the dedicated CT examinations for tumor measurements unless it is documented that the CT performed as part of the FDG PET-CT is of identical diagnostic quality to a diagnostic CT using intravenous (IV) and oral contrast.

### CT Scan Procedures

CT scans are acquired and submitted using consistent scan parameters (spacing, thickness, field of view, etc.) for all assessments with alternate imaging or parameter variation noted. If a patient develops hypersensitivity to the iodinated contrast medium while on study, it is acceptable to perform chest CT scans without contrast, and a contrast-enhanced MRI scan as mentioned herein must be performed for the remainder of the required anatomy. The patient should be given oral contrast (as either positive [eg, barium or Gastrografin] or negative [eg, water or saline] contrast media, according to site standard of care) prior to the examination to allow for sufficient bowel opacification. Non-ionic iodinated IV contrast with a minimum of 320 mg iodine/mL should be used and contrast agent volume should be according to the package insert. The same contrast agent with the same concentration is used.

### MRI Scan Procedures

If IV contrast is medically contraindicated during the study, a dynamic contrast-enhanced MRI is an acceptable alternative to CT scans of the abdomen and pelvis. An MRI must be performed using either 1.5 T or 3.0 T scanner. A change in scanner field strength is not allowed for an individual patient while on study (e.g., if a patient has a C2 MRI scan at 3.0 T, all subsequent scans must be performed at 3.0 T). MRI scans should use optimized parameters to decrease motion artifact and maximize signal-to-noise ratio and resolution. Breath-hold imaging, fast-scanning techniques, and gadolinium should be used to maximize lesion identification. A non-contrast CT scan of the chest or digital chest X-ray must be acquired in addition to the MRI of the abdomen and, as applicable, pelvis. The patient must undergo scanning with approved extracellular contrast media only (eg, Magnevist, Dataram, Omniscan). The same equipment, field strength, sequences, scanning parameters, positioning, angulation, timing, field of view, and slice thickness should be utilized for all examinations acquired both pre- and post-contrast for a given patient over the course of the study.

### Disease Response Assessments

Disease assessments are performed, and disease response assessed, every 8 weeks. In Phase 1 and Phase 2, responses are assessed; in Phase 2, assessments are confirmed by blinded radiographic review. Partial or complete response are confirmed by a repeat tumor imaging assessment no less than 4 weeks from the date the response was first documented. The tumor imaging for confirmation of response may be performed at the earliest, 4 weeks after the first indication of response, or at the next scheduled scan (ie, 8 weeks later), whichever is clinically indicated. When clinically stable, participants should not be discontinued until progression is confirmed.

TABLE 7
Disease Response Criteria for Target and Non-Target Lesions
Evaluation of Target Lesions

| | |
|---|---|
| Complete response (CR): | Disappearance of all target lesions |
| Partial response (PR): | At least a 30% decrease in the sum of the |
| | LD of target lesions, taking as reference the |
| | baseline sum LD |
| Progressive disease (PD): | At least a 20% increase in the sum of the |
| | LD of target lesions, taking as reference the |
| | smallest sum LD recorded since the |
| | treatment started or the appearance of one or |
| | more new lesions |
| Stable disease (SD): | Neither sufficient shrinkage to qualify for |
| | PR nor sufficient increase to qualify for PD, |
| | taking as reference the smallest sum LD |
| | since the treatment started |

Evaluation of nontarget lesions

| | |
|---|---|
| CR: | Disappearance of all target lesions |
| Non-CR/Non-PD: | Persistence of one or more nontarget |
| | lesion(s) |
| PD: | Appearance of one or more new lesions |
| | and/or unequivocal progression of existing |
| | nontarget lesions |

Abbreviation: LD = longest diameter.

### Tumor Tissue

If patients whose only accessible lesion for biopsy is a solitary target lesion, it must be amenable to a core biopsy that will not compromise assessment of tumor measurements. If patients have only one measurable lesion, then the biopsy specimen should be obtained from a non-target lesion, and the lesion should not have been in a field of prior-irradiation unless confirmed progression of the lesion.

ctDNA

Samples collected at screening and on treatment are analyzed for mutational landscape and changes in circulating tumor DNA (ctDNA) levels are evaluated for association with anti-tumor effect.

### Medical History, Including Cancer History, and Genotyping

A complete medical history is documented during Screening and updated at Baseline, before administration of the first Compound A dose. The medical history includes CRC history, including the patient's primary diagnosis, date of diagnosis, method of diagnosis, and all previous treatments, including radiation therapy, and response to such treatments. As part of the patient's cancer history, study centers submit a local histology or cytology report obtained before enrollment, if available. HIV history is to be documented. Mutational status from archival tests is to be documented. Archival flow cytometry reports are to be requested from the patient's local institution and collected, if possible.

### Physical Examinations

A complete physical examination is conducted for all patients during Screening and at the Safety Follow-up visit. The complete physical examination includes general appearance, head/ears/eyes/nose/throat, lungs/chest, heart, abdomen, lymph nodes, musculoskeletal, extremities, and neurological examinations.

### Electrocardiograms

All ECGs are performed in triplicate with each separated by 2 minutes. QTc is calculated using Fridericia's correction (QTcF), if necessary. The average of the 3 ECGs is used to calculate QTcF. If a patient experiences ≥Grade 2 QTcF prolongation, a clinical chemistry sample (for magnesium, potassium, and calcium) is obtained, and for ≥Grade 3 QTcF prolongation (based on the average of triplicate ECGs), a single contemporaneous PK sample is obtained as soon as possible, and the time of the last dose of study drug is recorded. It is recommended that patients who are experiencing vomiting or diarrhea have their electrolytes checked and have more frequent ECGs. An ECG should be obtained before and after beginning any new medication and after dose increases of concurrent medications known to prolong QTc.

All ECGs collected should initially be locally read to enable real-time treatment decisions.

### Time Period and Frequency for Collecting AE and SAE Information

All AEs, including SAEs, are collected from the time of informed consent until the Safety Follow-up visit 30 days after the last Compound A dose. Medical occurrences that begin before the start of Compound A, but after obtaining informed consent, are recorded. All SAEs are recorded and reported. Any SAE, including a death, at any time after a participant has been discharged from the study, that is related to the study drug or study participation is reported.

### Pharmacokinetics of Compound A

Blood samples are collected for measurement of plasma concentrations of Compound A (PK). Samples of blood are centrifuged to prepare plasma. Samples collected for analyses of Compound A concentration may also be used to evaluate safety or efficacy aspects related to concerns arising during or after the study.

### Survival Follow-Up

All patients receive survival follow-up calls: These occur monthly for 11 months after the safety follow-up visit, and then the frequency will change to every 3 months. The date of follow-up is recorded. The patient status is collected until the patient is lost to follow-up, withdrawal of consent, or death. The date of death is recorded. Additionally, any new cancer therapy the patient has started after study treatment termination is recorded.

### Study Termination

Upon termination of the patient's participation in the study, the date and reason for study termination are recorded.

### EQUIVALENTS

The details of one or more embodiments of the disclosure are set forth in the accompanying description above. Although any methods and materials similar or equivalent to those described herein can be used in the practice or testing of the present disclosure, the preferred methods and materials are now described. Other features, objects, and advantages of the disclosure will be apparent from the description and from the claims. In the specification and the appended claims, the singular forms include plural referents unless the context clearly dictates otherwise. Unless defined otherwise, all technical and scientific terms used herein have the same meaning as commonly understood by one of ordinary skill in the art to which this disclosure belongs. All patents and publications cited in this specification are incorporated by reference.

The foregoing description has been presented only for the purposes of illustration and is not intended to limit the disclosure to the precise form disclosed, but by the claims appended hereto.

What is claimed is:

1. A method of treating, preventing, or reducing the severity of a colorectal cancer in a subject in need thereof, comprising administering to the subject a menin-MLL inhibitor or a pharmaceutical composition comprising a menin-MLL inhibitor.

2. A method of treating, preventing, or reducing the severity of a colorectal cancer in a subject in need thereof, comprising administering to the subject a pharmaceutical composition consisting essentially of a menin-MLL inhibitor.

3. A method of treating, preventing, or reducing the severity of a colorectal cancer in a subject in need thereof consisting essentially of administering to the subject a pharmaceutical composition comprising a menin-MLL inhibitor.

4. A method of treating, preventing, or reducing the severity of colorectal cancer in a subject in need thereof, comprising administering to the subject a single anti-cancer agent, wherein the single anti-cancer agent is a menin-MLL inhibitor.

5. The method of any one of the preceding claims, wherein the menin-MLL inhibitor is Compound A, Compound B, DSP-5336, DS-1594, KO-539, MI-503, MI-3454, MI-463, M-808, MI-136, JNJ-75276617, MI-227, BMF-219, the antibody A300-105A, MI-0202, MI-503, MI-463, MI-136, ML-227, MI-2-2, or ISC-30.

6. The method of any one of the preceding claims, wherein the menin-MLL inhibitor is Compound A or Compound B.

7. The method of any one of the preceding claims, wherein the menin-MLL inhibitor is Compound A.

8. The method of any one of the preceding claims, wherein the menin-MLL inhibitor is administered at least once daily.

9. The method of any one of the preceding claims, wherein the menin-MLL inhibitor is administered twice daily.

10. The method of any one of the preceding claims, wherein the menin-MLL inhibitor is administered three times daily.

11. The method of any one of the preceding claims, wherein the method further comprises administering a CYP3A inhibitor.

12. The method of any one of the preceding claims, wherein the method further comprises administering a CYP3A4 inhibitor.

13. The method of claim 12, wherein the CYP3A4 inhibitor is a strong CYP3A4 inhibitor.

14. The method of claim 13, wherein the strong CYP3A4 inhibitor is boceprevir, cobicistat, clarithromycin, conivaptan, grapefruit juice, indinavir, itraconazole, ketoconazole, lopinavir, mibefradil, nefazodone, nelfinavir, posaconazole, ritonavir, saquinavir, telaprevir, telithromycin, or voriconazole.

15. The method of any one of claims 1-10, wherein the method further comprises administering cobicistat.

16. The method of any one of claims 1-10, wherein the method further comprises administering cobicistat at least once daily.

17. The method of any one of claims 1-10, wherein the method further comprises administering cobicistat once daily.

18. The method of claim 12, wherein the CYP3A4 inhibitor is a moderate CYP3A4 inhibitor.

19. The method of claim 18, wherein the moderate CYP3A4 inhibitor is amprenavir, aprepitant, atazanavir, ciprofloxacin, darunavir, diltiazem, erythromycin, fluconazole, fosamprenavir, imatinib, or verapamil.

20. The method of any one of the preceding claims, wherein the menin-MLL inhibitor is administered at a dosage of between about 25 mg and about 276 mg every 12 hours.

21. The method of any one of the preceding claims, wherein the menin-MLL inhibitor is administered at a dosage of about 163 mg every 12 hours.

22. The method of any one of claims 1-10, wherein the subject does not receive a CYP3A inhibitor.

23. The method of any one of claims 1-10, wherein the pharmaceutical composition does not comprise a CYP3A inhibitor.

24. The method of either claim 22 or 23, wherein the menin-MLL inhibitor is administered at a dosage of between about 25 mg and about 276 mg.

25. The method of either claim 22 or 23, wherein the menin-MLL inhibitor is administered at a dosage of between about 25 mg and about 276 mg, and wherein the menin-MLL inhibitor is administered three times per day.

26. The method of either claim 22 or 23, wherein the menin-MLL inhibitor is administered at a dosage of about 276 mg.

27. The method of either claim 22 or 23, wherein the menin-MLL inhibitor is administered at a dosage of about 276 mg, and wherein the menin-MLL inhibitor is administered three times per day.

28. The method of any one of claims 1-19, 22-25, and 27, wherein the menin-MLL inhibitor is administered at a dosage of up to about 828 mg per day.

29. The method of any one of the preceding claims, wherein the menin-inhibitor is contained within a capsule or oral solution.

30. The method of any one of the preceding claims, wherein the subject previously received at least one prior line of therapy for colorectal cancer.

31. The method of any one of the preceding claims, wherein the subject previously received at least one prior line of therapy for colorectal cancer, and wherein the subject did not respond to previous therapy.

32. The method of either claim 30 or 31, wherein the at least one prior line of therapy was a chemotherapy, radiation therapy, immunotherapy, targeted small molecule therapy or biologic therapy (e.g., monoclonal antibody therapy).

33. The method of any one of the preceding claims, wherein the subject has previously received a treatment for colorectal cancer selected from trifluridine, tipiracil and or regorafenib.

34. The method of any one of the preceding claims, wherein the subject has previously received trifluridine, tipiracil, or a combination thereof.

35. The method of any one of the preceding claims, wherein the subject has previously received regorafenib.

36. The method of any one of the preceding claims, wherein the subject progressed on at least one prior line of therapy for colorectal cancer.

37. The method of any one of the preceding claims, wherein the subject is being treated for microsatellite stable/proficient mismatch repair (MSS/pMMR) mCRC.

38. The method of any one of the preceding claims, wherein subject is not receiving an EGFR inhibitor.

39. A method of treating, preventing, or reducing the severity of a colorectal cancer in a subject in need thereof comprising administering:
a pharmaceutical composition comprising Compound A at least once daily,
a pharmaceutical composition comprising cobicistat at least once daily, and
wherein the subject is not receiving an EGFR inhibitor.

40. A method of treating, preventing, or reducing the severity of a colorectal cancer in a subject in need thereof comprising administering Compound A at a dose between 150 mg and 200 mg dose three times daily, wherein the subject does not receive a CYP3A inhibitor and an EGFR inhibitor.

41. A method of any one of the preceding claims, wherein the colorectal cancer is characterized by dysregulation of the WNT/β-catenin signaling pathway.

42. A method of any one of the preceding claims, wherein the subject has progressed on a prior colorectal cancer therapy due to developing resistance to the prior colorectal cancer therapy.

43. A method of treating, preventing, or reducing the severity of a cancer in a subject comprising administering a pharmaceutical composition comprising Compound A, wherein the method comprises administering from about 220 to about 280 mg of Compound A three times per day, wherein the subject does not receive a CYP3A inhibitor.

44. The method of claim 43, wherein the cancer is a leukemia.

45. The method of claim 44, wherein the cancer is an AML leukemia.

46. The method of claim 44, wherein the cancer is an acute lymphoblastic leukemia.

47. The method of claim 44, wherein the cancer is an NMP1-mutated leukemia.

48. The method of claim 43, wherein the cancer is a colorectal cancer.

49. The method of claim 43, wherein the cancer is a microsatellite stable/proficient mismatch repair (MSS/pMMR) mCRC.

50. The method of any one of claims 43-49, wherein the menin-MLL inhibitor is administered at a dosage of about 270 mg to about 280 mg.

51. The method of any one of claims 43-50, wherein the menin-MLL inhibitor is administered three times per day.

52. The method of any one of the preceding claims, wherein the menin-MLL inhibitor is administered with food.

53. A menin-MLL inhibitor for use in the preparation of a medicament for the treatment of colorectal cancer according to the method of any one of the previous claims, wherein the medicament and/or treatment does not involve the use of an EGFR inhibitor.

54. A menin-MLL inhibitor for use in the preparation of a medicament for the treatment of colorectal cancer according to the method of any one of the previous claims, wherein the medicament does not comprise an EGFR inhibitor.

55. A kit comprising a menin-MLL inhibitor, and printed instructions for using the menin-MLL inhibitor in a subject for the treatment for colorectal cancer.

56. A method of treating, preventing, or reducing the severity of a cancer in a subject in need thereof, comprises administering to the subject Compound A, or a pharmaceutically acceptable salt thereof, or a pharmaceutical composition comprising Compound A, wherein Compound A is administered at a dosage of between about 163 mg and about 276 mg every 8 hours, wherein upon administration, the area under plasma concentration versus time curve from time 0 to 24 hours (AUC₍₀₋₂₄₎) of Compound A is from about 1,000 ng*h/mL to about 100,000 ng*h/mL.

57. The method of claim 56, wherein Compound A, or the pharmaceutically acceptable salt thereof, or the pharmaceutical composition comprising Compound A is administered at a dosage of about 163 mg every 8 hours, wherein upon administration the area under plasma concentration versus time curve from time 0 to 24 hours (AUC₍₀₋₂₄₎) of Compound A is from about 10,000 ng*h/mL to about 60,000 ng*h/mL.

58. The method of claim 56, wherein Compound A, or the pharmaceutically acceptable salt thereof, or the pharmaceutical composition comprising Compound A is administered at a dosage of about 226 mg every 8 hours, wherein upon administration the area under plasma concentration versus time curve from time 0 to 24 hours (AUC₍₀₋₂₄₎) of Compound A is from about 14,000 ng*h/mL to about 65,000 ng*h/mL.

59. The method of claim 56, wherein Compound A, or the pharmaceutically acceptable salt thereof, or the pharmaceutical composition comprising Compound A is administered at a dosage of about 276 mg every 8 hours, wherein upon administration the area under plasma concentration versus time curve from time 0 to 24 hours (AUC₍₀₋₂₄₎) of Compound A is from about 17,000 ng*h/mL to about 90,000 ng*h/mL.

60. The method of claim 56, wherein Compound A, or the pharmaceutically acceptable salt thereof, or the pharmaceutical composition comprising Compound A is administered at a dosage of about 163 mg every 8 hours, wherein upon administration the area under plasma concentration versus time curve from time 0 to 24 hours (AUC₍₀₋₂₄₎) of Compound A is about 22,000 ng*h/mL.

61. The method of claim 56, wherein Compound A, or the pharmaceutically acceptable salt thereof, or the pharmaceutical composition comprising Compound A is administered at a dosage of about 226 mg every 8 hours, wherein upon administration the area under plasma concentration versus time curve from time 0 to 24 hours (AUC₍₀₋₂₄₎) of Compound A is about 30,500 ng*h/mL.

62. The method of claim 56, wherein Compound A, or the pharmaceutically acceptable salt thereof, or the pharmaceutical composition comprising Compound A is administered at a dosage of about 276 mg every 8 hours, wherein upon administration the area under plasma concentration versus time curve from time 0 to 24 hours (AUC₍₀₋₂₄₎) of Compound A is about 41,000 ng*h/mL.

63. A method of treating, preventing, or reducing the severity of a cancer in a subject in need thereof, comprises administering to the subject Compound A, or a pharmaceutically acceptable salt thereof, or a pharmaceutical composition comprising Compound A, wherein Compound A is administered at a dosage from about 163 mg to about 276 mg every 8 hours, and wherein upon administration the minimum observed concentration (Cₘᵢₙ) of Compound A is from about 100 ng/ml to about 2,400 ng/mL.

64. The method of claim 63, wherein Compound A, or the pharmaceutically acceptable salt thereof, or the pharmaceutical composition comprising Compound A is administered at a dosage of about 163 mg every 8 hours, wherein upon administration the minimum observed concentration (Cₘᵢₙ) of Compound A is from about 100 ng/ml to about 1,400 ng/mL.

65. The method of claim 63, wherein Compound A, or the pharmaceutically acceptable salt thereof, or the pharmaceutical composition comprising Compound A is administered at a dosage of about 226 mg every 8 hours, wherein upon administration the minimum observed concentration (Cₘᵢₙ) of Compound A is from about 100 ng/ml to about 1,600 ng/mL.

66. The method of claim 63, wherein Compound A, or the pharmaceutically acceptable salt thereof, or the pharmaceutical composition comprising Compound A is administered at a dosage of about 276 mg every 8 hours, wherein upon administration the minimum observed concentration (Cₘᵢₙ) of Compound A is from about 100 ng/ml to about 2,400 ng/mL.

67. The method of claim 63, wherein Compound A, or the pharmaceutically acceptable salt thereof, or the pharmaceutical composition comprising Compound A is administered at a dosage of about 163 mg every 8 hours, wherein upon administration the minimum observed concentration (Cₘᵢₙ) of Compound A is about 400 ng/ml.

68. The method of claim 63, wherein Compound A, or the pharmaceutically acceptable salt thereof, or the pharmaceutical composition comprising Compound A is administered at a dosage of about 226 mg every 8 hours, wherein upon administration the minimum observed concentration (Cₘᵢₙ) of Compound A is about 500 ng/mL.

69. The method of claim 63, wherein Compound A, or the pharmaceutically acceptable salt thereof, or the pharmaceutical composition comprising Compound A is administered at a dosage of about 276 mg every 8 hours, wherein upon administration the minimum observed concentration (Cₘᵢₙ) of Compound A is about 800 ng/mL.

70. A method of treating, preventing, or reducing the severity of a cancer in a subject in need thereof, comprises administering to the subject Compound A, or a pharmaceutically acceptable salt thereof, or a pharmaceutical composition comprising Compound A, wherein Compound A is administered at a dosage from about 163 mg to about 276 mg every 8 hours, and wherein upon administration the maximum observed concentration (Cₘₐₓ) of Compound A is from about 1,000 ng/ml to about 6,000 ng/ml.

71. The method of claim 70, wherein Compound A, or the pharmaceutically acceptable salt thereof, or the pharmaceutical composition comprising Compound A is administered at a dosage of about 163 mg every 8 hours, wherein upon administration the maximum observed concentration (Cₘₐₓ) of Compound A is from about 1,000 ng/mL to about 3,400 ng/mL.

72. The method of claim 70, wherein Compound A, or the pharmaceutically acceptable salt thereof, or the pharmaceutical composition comprising Compound A is administered at a dosage of about 226 mg every 8 hours, wherein upon administration the maximum observed concentration (Cₘₐₓ) of Compound A is from about 1,300 ng/ml to about 4,400 ng/mL.

73. The method of claim 70, wherein Compound A, or the pharmaceutically acceptable salt thereof, or the pharmaceutical composition comprising Compound A is administered at a dosage of about 276 mg every 8 hours, wherein upon administration the maximum observed concentration (Cₘₐₓ) of Compound A is from about 1,700 ng/ml to about 5,400 ng/mL.

74. The method of claim 70, wherein Compound A, or the pharmaceutically acceptable salt thereof, or the pharmaceutical composition comprising Compound A is administered at a dosage of about 163 mg every 8 hours, wherein upon administration maximum observed concentration (Cₘₐₓ) of Compound A is about 1700 ng/ml.

75. The method of claim 70, wherein Compound A, or the pharmaceutically acceptable salt thereof, or the pharmaceutical composition comprising Compound A is administered at a dosage of about 226 mg every 8 hours, wherein upon administration the maximum observed concentration (Cₘₐₓ) of Compound A is about 2400 ng/mL.

76. The method of claim 70, wherein Compound A, or the pharmaceutically acceptable salt thereof, or the pharmaceutical composition comprising Compound A is administered at a dosage of about 276 mg every 8 hours, wherein upon administration the maximum observed concentration (Cₘₐₓ) of Compound A is about 3,100 ng/mL.

77. A method of treating, preventing, or reducing the severity of a cancer in a subject in need thereof, comprises administering to the subject Compound A, or a pharmaceutically acceptable salt thereof, or a pharmaceutical composition comprising Compound A and a CYP3A4 inhibitor, wherein Compound A is administered at a dosage of about 163 mg every 12 hours, and wherein upon administration the area under plasma concentration versus time curve from time 0 to 24 hours (AUC₍₀₋₂₄₎) of Compound A is from about 19,000 ng*h/mL to about 110,000 ng*h/mL.

78. The method of claim 77, wherein Compound A is administered at a dosage of about 163 mg every 12 hours, and wherein upon administration the area under plasma concentration versus time curve from time 0 to 24 hours (AUC₍₀₋₂₄₎) of Compound A is about 43,000 ng*h/mL.

79. A method of treating, preventing, or reducing the severity of a cancer in a subject in need thereof, comprises administering to the subject Compound A, or a pharmaceutically acceptable salt thereof, or a pharmaceutical composition comprising Compound A and a CYP3A4 inhibitor, wherein Compound A is administered at a dosage of about 163 mg every 12 hours, and wherein upon administration the minimum observed concentration (Cₘᵢₙ) of Compound A is from about 200 ng/ml to about 2,800 ng/mL.

80. The method of claim 79, wherein Compound A is administered at a dosage of about 163 mg every 12 hours, and wherein upon administration the minimum observed concentration (Cₘᵢₙ) of Compound A is about 900 ng/ml.

81. A method of treating, preventing, or reducing the severity of a cancer in a subject in need thereof, comprises administering to the subject Compound A, or a pharmaceutically acceptable salt thereof, or a pharmaceutical composition comprising Compound A and a CYP3A4 inhibitor, wherein Compound A is administered at a dosage of about 163 mg every 12 hours, and wherein upon administration the maximum observed concentration (Cₘₐₓ) of Compound A is from about 1,700 ng/ml to about 6,200 ng/mL.

82. The method of claim 81, wherein Compound A is administered at a dosage of about 163 mg every 12 hours, and wherein upon administration the maximum observed concentration (Cₘₐₓ) of Compound A is about 3,200 ng/mL.
